(19) United States
(12) Patent Application Publication
(10) Pub. No.: US 2026/0335081 A1
(43) Pub. Date: Oct. 29, 2026

(54) Optical Sensing for Leaflet Grasping and Transcatheter Edge-to-Edge Repair

(71) Applicant: Evalve, Inc., Santa Clara, CA (US)

(72) Inventors: Michael F. Wei, Redwood, CA (US); Michael C. Nasr, Benecia, CA (US); Jarad Waisblatt, San Jose, CA (US); Michael Shin, Pleasanton, CA (US); Manish Gada, San Jose, CA (US); Anna Maria Snell, Belmont, CA (US); Chad J. Abunassar, Alameda, CA (US); Fermin A. Lupotti, Lake Forest, CA (US)

(73) Assignee: Evalve, Inc., Santa Clara, CA (US)

(21) Appl. No.: 19/660,143

(22) Filed: Apr. 27, 2026

Related U.S. Application Data
(60) Provisional application No. 63/795,720, filed on Apr. 28, 2025.

(51) Int. Cl. A61F 2/24

(52) U.S. Cl. CPC A61F 2/2466; A61F 2/246

(57) ABSTRACT

An interventional system that includes a delivery catheter that has a proximal end portion and a distal end portion. The interventional system includes a fixation device that has a distal fixation element, one or more reflective markers, and a proximal fixation element. The proximal fixation element is actuatable between an open position defining a graspable volume of space between the proximal fixation and the distal fixation element and a closed position. In the closed position the proximal fixation element is in a position proximal to the distal fixation element. The interventional system includes a sensor assembly that has a signal emitter configured to direct an emitted signal along an emission path toward the one or more reflective markers and a signal receiver configured to receive a reflected signal of the emitted signal off of the one or more reflective markers.

### CROSS REFERENCE TO RELATED APPLICATIONS

This U.S. Patent Application claims priority under 35 U.S.C. § 119(e) to U.S. Provisional Application 63/795,720, filed on Apr. 28, 2025. The disclosure of this prior application is considered part of the disclosure of this application and is hereby incorporated by reference in its entirety.

### TECHNICAL FIELD

This disclosure relates to infrared sensing for improved leaflet grasping and transcatheter edge-to-edge repair outcomes.

### BACKGROUND

The cardiac cycle is divided into two phases-diastole and systole. Diastole is generally characterized by the muscular relaxation of the heart and the filling of its chambers with blood. On the other hand, systole is generally characterized by the muscular contraction of the ventricles which pumps blood from the ventricles to the arteries. During ventricular systole, ventricular pressure increases relative to atrial pressure resulting in the closure of the mitral valve and the tricuspid valve. The mitral valve separates the left atrium from the left ventricle, and the tricuspid valve separates the right atrium from the right ventricle. These valves operate as check valves preventing blood from flowing back into the atria during ventricular contraction. However, valvular insufficiency may appear in one or both of these valves which may result in a regurgitative flow back into the atrium across the effected valve. Such regurgitative flow can be in the form of mitral valve regurgitation (“MVR”) and/or tricuspid valve regurgitation (“TVR”). Left untreated, MVR and TVR can lead to severe health consequences, such as progressive heart failure, cardiac arrythmias, pulmonary hypertension, stroke, and endocarditis, to name a few.

MVR and TVR can have a variety of etiologies which typically fall into the categories of degenerative (primary) and functional (secondary) regurgitation.

Degenerative valve regurgitation principally occurs due to abnormalities or degeneration of the valve apparatus, such as the valve leaflets, valve annulus, chordae tendineae, and/or papillary muscles. One example of a degenerative valve condition is mitral valve prolapse. Functional valve regurgitation is often a secondary condition that arises from underlying heart conditions or diseases that affect the structure or function of the heart.

Examples of conditions that can result in functional regurgitation include dilated cardiomyopathy, ischemic heart disease, pulmonary hypertension, and heart failure.

Regardless of the underlying condition precipitating the regurgitative flow, the primary mechanism by which regurgitation occurs is the failure of the valve leaflets to properly and completely seal or coapt during systole which allows a jet of blood to flow back into the atrium between the effected leaflets.

Treatment options for MVR and TVR generally include Guideline-Directed Medical Therapy (“GDMT”), valve replacement, and valve repair. GDMT usually involves the administration of a combination of drugs that treat an underlying heart condition. Valve replacement and repair may include open-heart surgical options and catheter-based options. Catheter-based repair procedures are sometimes referred to as transcatheter edge-to-edge repair (“TEER”).

### SUMMARY

One aspect of the disclosure provides an interventional system for repairing a heart valve. The interventional system includes a delivery catheter that has a proximal end portion and a distal end portion. The interventional system includes a fixation device releasably coupled to the distal end portion of the delivery catheter. The fixation device includes a distal fixation element having an engagement surface and is actuatable between an open position and a closed position. The fixation device includes one or more reflective markers disposed on the engagement surface of the distal fixation element. The fixation device also includes a proximal fixation element that has an engagement surface and is actuatable between an open position defining a graspable volume of space between the engagement surface of the proximal fixation element and the engagement surface of the distal fixation element and a closed position. In the closed position the engagement surface of the proximal fixation element is in a position proximal to the engagement surface of the distal fixation element. The interventional system includes a sensor assembly disposed on the distal end portion of the delivery catheter. The sensor assembly includes a signal emitter configured to direct an emitted signal along an emission path toward one or more reflective markers disposed on the engagement surface of the distal fixation element when in the open position and a signal receiver configured to receive a reflected signal of the emitted signal off of the one or more reflective markers. Advantageously, this combination of features allows for real-time, objective confirmation of leaflet insertion depth prior to device closure, overcoming the visualization challenges of a dynamic, blood-filled cardiovascular environment. By utilizing the sensor assembly to detect the progressive occlusion of the reflective markers, a clinician may precisely verify that a leaflet is sufficiently seated within the graspable volume of space, thereby improving grasping outcomes, reducing the likelihood of single leaflet device attachment (SLDA), and minimizing the need for procedural repositioning.

Implementations of the disclosure may include one or more of the following optional features. In some implementations, the one or more reflective markers are arranged such that the reflected signal is proportional to an amount of occlusion of the one or more reflective markers by a leaflet of the heart valve positioned in the graspable volume of space. The engagement surface of the distal fixation element may have a proximal region and a distal region. The one or more reflective markers are arranged in a pattern along the engagement surface of the distal fixation element from the proximal region to the distal region. This arrangement beneficially enables the system to differentiate between a marginally inserted leaflet located only at the distal region and a deeply, securely seated leaflet reaching the proximal region. In some examples, the one or more reflective markers are arranged such that the proximal fixation element fails to obstruct the emission path toward the one or more reflective markers. By maintaining an unimpeded line of sight, this configuration ensures continuous monitoring of the graspable volume of space during the entire grasping sequence, even as the proximal fixation element is being actuated. The emitted signal may include infrared light and the one or more reflective markers may be configured to reflect infrared light.

Advantageously, infrared light penetrates blood and surrounding fluid more effectively than visible light, facilitating highly reliable leaflet detection in a dynamic cardiovascular environment without interference from ambient light sources or continuous blood flow.

In some implementations, the signal emitter is configured to emit near-infrared light having a wavelength of about 700 nm to about 900 nm or about 1000 nm to about 1700 nm. The one or more reflective markers may include a biocompatible material selected from the group consisting of gold, titanium nitride, and alumina with a metallic coating. Beneficially, these specific materials provide exceptional optical reflectivity in the targeted infrared spectrum while maintaining the strict biocompatibility, corrosion resistance, and durability required for long-term cardiovascular implantation.

In some examples, the interventional system further includes a covering disposed over at least a portion of the engagement surface of the distal fixation element. Here, the one or more reflective markers at least partially protrude through the covering.

In some implementations, the interventional system of further includes a feedback indicator and a controller in communication with the sensor assembly and the feedback indicator. The controller includes data processing hardware and memory hardware storing instructions that when executed by the data processing hardware cause the data processing hardware to receive signal data from the sensor assembly corresponding to the reflected signal, determine an insertion characteristic indicative of a position of a leaflet of the heart valve in the graspable volume of space, based on the signal data, and present a representation of the insertion characteristic by the feedback indicator. Advantageously, this automated processing translates raw, fluctuating optical signals into a stable, easily interpretable metric, significantly reducing the cognitive load on the clinician and enabling rapid, objective decision-making during a time-sensitive procedure. In these implementations, the representation of the insertion characteristic includes at least one of a visual representation or a haptic representation.

Another aspect of the disclosure provides an interventional system for repairing a heart valve. The interventional system includes a delivery catheter that has a proximal end portion and a distal end portion. The interventional system includes a fixation device releasably coupled to the distal end portion of the delivery catheter. The fixation device includes a proximal fixation element and a distal fixation element. The proximal fixation element and the distal fixation element are actuatable between an open position and a closed position for grasping a leaflet of the heart valve therebetween. The interventional system also includes a sensor assembly disposed on the distal end portion of the delivery catheter. The sensor assembly includes a signal emitter arranged to direct an emitted signal along an emission path toward a graspable volume of space defined by the proximal fixation element and the distal fixation element when in the open position and a signal receiver arranged to receive a reflected signal of the emitted signal off of the leaflet of the heart valve when positioned within the graspable volume of space. This direct-tissue sensing configuration is particularly advantageous because it provides real-time positional feedback based on the physical presence of the tissue itself, eliminating the need for specialized reflective markers on the implant. This simplifies device manufacturing, reduces the overall profile of the fixation elements, and makes the detection system less sensitive to the specific orientation of the leaflet within the capture zone.

Implementations of the disclosure may include one or more of the following optional features. In some implementations, the reflected signal is proportional to a position of the leaflet within the graspable volume of space. In some examples, the signal emitter is configured to emit infrared light and the signal receiver is configured to receive reflected infrared light from the leaflet. This is advantageous because leaflet tissue reflects infrared light at a significantly higher intensity than the surrounding blood pool, providing the necessary optical contrast to reliably detect the soft tissue despite the presence of flowing blood. The signal emitter may be configured to emit near-infrared light that has a wavelength of about 700 nm to about 900 nm or about 1000 nm to about 1700 nm. Operating within these specific wavelength ranges is highly beneficial as it targets optimal optical windows where interference from hemoglobin and water absorption is minimized, thereby maximizing signal clarity and detection accuracy.

In some implementations, the interventional system further includes a feedback indicator and a controller in communication with the sensor assembly and the feedback indicator. The controller includes data processing hardware and memory hardware storing instructions that when executed by the data processing hardware cause the data processing hardware to receive signal data from the sensor assembly corresponding to the reflected signal, determine an insertion characteristic indicative of a position of the leaflet in the graspable volume of space based on the signal data, and present a representation of the insertion characteristic by the feedback indicator. By actively filtering and analyzing the direct-tissue reflections, this configuration advantageously ensures that the clinician is provided with a reliable, actionable metric of leaflet depth, mitigating the risk of a partial grasp and reducing the likelihood of requiring subsequent device repositioning. Here, the representation of the insertion characteristic may include at least one of a visual representation or a haptic representation.

Another aspect of the disclosure provides a method of detecting leaflet insertion during a transcatheter edge-to-edge repair procedure. The method includes positioning a fixation device at a leaflet of a heart valve within a heart. The fixation device includes a distal fixation element having an engagement surface and is actuatable between an open position and a closed position and a proximal fixation element having an engagement surface and is actuatable between an open position and a closed position. The method includes actuating the distal fixation element and the proximal fixation element to the open position and emitting a signal from a sensor assembly toward the engagement surface of the distal fixation element. The method also includes receiving, by the sensor assembly, signal data corresponding to a reflected signal of the emitted signal. The method includes determining an insertion characteristic of the leaflet relative to the distal fixation element based on the signal data and presenting a representation of the insertion characteristic by a feedback indicator.

Implementations of the disclosure may include one or more of the following optional features. The method may include actuating the proximal fixation element toward the closed position to grasp the leaflet. In some implementations, the method further includes identifying a baseline for the sensor assembly by actuating the distal fixation element to the closed position to confirm an absence of the reflected signal and actuating the distal fixation element to the open position to confirm a presence of the reflected signal. The method may further include adjusting at least one of an orientation or a position of the distal fixation element until the insertion characteristic satisfies a threshold. In some examples, the method further includes actuating the proximal fixation element toward the open position and repositioning the fixation device relative to the leaflet based on the insertion characteristic indicating insufficient leaflet insertion. The representation of the insertion characteristic may include at least one of a visual representation or a haptic representation.

In some implementations, presenting the representation of the insertion characteristic includes displaying at least one of a first color indicating a threshold insertion of the leaflet, a second color indicating marginal insertion of the leaflet, or a third color indicating no insertion of the leaflet. Presenting the representation of the insertion characteristic may include displaying a numeric value. In some examples, the fixation device further includes one or more reflective markers disposed on the engagement surface of the distal fixation element and the sensor assembly includes a signal emitter and a signal receiver. The signal emitter is configured to direct the emitted signal along an emission path toward the one or more reflective markers disposed on the engagement surface of the distal fixation element when in the open position. The signal receiver is configured to receive the reflected signal of the emitted signal off of the one or more reflective markers. Here, determining the insertion characteristic includes interpreting a decrease in the reflected signal as an increase in leaflet insertion relative to the engagement surface of the distal fixation element. In some implementations, the sensor assembly includes a signal emitter arranged to direct the emitted signal along an emission path toward a graspable volume of space defined by the proximal fixation element and the distal fixation element when in the open position and a signal receiver arranged to receive the reflected signal of the emitted signal off of the leaflet of the heart valve when positioned within the graspable volume of space. Here, determining the insertion characteristic includes interpreting an increase in the reflected signal as an increase in leaflet insertion relative to the distal fixation element.

In another aspect of the present disclosure, a fixation device includes a first clamp and a second clamp. The first clamp includes a first distal element and a first proximal element. The first distal element and the first proximal element are each moveable relative to each other and each having a free end that defines an open end of the first clamp and a fixed end that at least partially defines a closed end of the first clamp. The second clamp has a second distal element and a second proximal element.

The second distal element and the second proximal element are moveable relative to each other and each have a free end that defines an open end of the second clamp and a fixed end that at least partially defines a closed end of the second clamp.

The fixation device also includes a first optical sensor disposed proximate the closed end of the first clamp. The first optical sensor has a first emitter and a first detector. The first emitter is configured to emit light in the infrared (IR) or near infrared (NIR) spectrum into a space defined between the first proximal element and the first distal element and in a direction toward the open end of the first clamp. The first detector is configured to detect light returning from the space in a direction toward the closed end of the first clamp and to generate a signal indicative of one or more characteristics of a first tissue disposed within the first clamp.

In one or more implementations of the fixation device, the one or more characteristics may include a distance between the first optical sensor and the first tissue. In one or more implementations of the fixation device, the one or more characteristics may include tissue type.

In one or more implementations of the fixation device, a first emitter may be configured to direct one or more beams of light toward the first proximal element such that the one or more beams of light intersect the first proximal element at one or more discrete locations. For example, the first emitter may include a light source that is one of a light-emitting diode (LED) and a laser diode. In some examples, the first emitter may be configured to emit light with a wavelength selected from the range of 1,000 to 3,000 nm. In other examples, the first emitter may be configured to emit light with a wavelength selected from the range of 700 to 1,500 nm. Also, in some examples, the first emitter may include an optical element. For example, the optical element may be any one or more of a beam splitter, a diffuser, and a lens.

In one or more implementations of the fixation device, the first detector may be configured to detect light reflected back from the one or more discrete locations. For example, first detector may include a detector component that includes one of a photodiode, phototransistor, charge-coupled device (CCD), complementary metal-oxide-semiconductor (CMOS), and photomultiplier tube (PMT). In examples in which the first detector is a photodiode, the photodiode may have a near portion and a far portion.

Additionally, in some examples, the signal generated by the detector may be a dynamic signal based on optical interactions between the one or more beams of light and at least one of the first proximal element and the first tissue. Also, in some examples, the first detector may include an optical element. For example, the optical element may be any one or more of a lens, a beam splitter, a prism, and a diffuser.

In one or more implementations of the fixation device, the one or more beams of light may include a first beam that intersects the first proximal element at a first discrete location of the one or more discrete locations, and a second beam that intersects the proximal element at a second discrete location of the one or more discrete locations. In some examples, first discrete location may be located beyond 50% of a grip length of the first proximal element as measured from the free end thereof. In other examples, the second discrete location may be at 50% of the grip length of the first proximal element.

In further examples, the second discrete location may be located between the free end and 50% of the grip length of the first proximal element.

In one or more implementations of the fixation device, the first emitter may be configured to emit a third beam of light intersecting the first proximal element at a third discrete location of the one or more discrete locations. In some examples, the third discrete location may be located between the free end and 50% of the grip length of the first proximal element.

In one or more implementations of the fixation device, the first detector and the first emitter may be positioned within the closed end of the first clamp and oriented at an angle towards the first proximal element.

In one or more implementations of the fixation device, the first emitter may be oriented parallel to the first distal element when the first distal element is in an open position.

In one or more implementations of the fixation device, the first detector may be oriented in parallel with the first emitter.

In one or more implementations of the fixation device, the first detector may be oriented at an non-zero angle relative to the first emitter.

In one or more implementations of the fixation device, a second optical sensor may be disposed at the closed end of the second clamp, the second optical sensor having a second emitter and a second detector, the second emitter being configured to emit light in the infrared (IR) or near infrared (NIR) spectrum into a space defined between the second proximal element and the second distal element and in a direction toward the open end of the second clamp, and the second detector being configured to detect light returning from the space in a direction toward the closed end of the second clamp and to generate a signal indicative of one or more characteristics of tissue disposed within the second clamp.

In another aspect of the present disclosure, a fixation device includes a first clamp and a second clamp. The first clamp includes a first distal element and a first proximal element. The first distal element and the first proximal element each have a fixed end, a free end, and a length defined between the free end and the fixed end. The second clamp has a second distal element and a second proximal element. The second distal element and the second proximal element each have a fixed end, a free end, and a length defined between the free end and the fixed end.

The fixation device also includes a first optical sensor. The first optical sensor includes a first emitter and a first detector. The first emitter or the first detector is coupled to the first proximal element, and the other of the first emitter and the first detector is coupled to the first distal element such that the first emitter is positioned directly opposite the first detector. When a first tissue is absent from the first clamp, light emitted from the first emitter is received by the first detector which is configured to generate a first signal indicative of absent tissue. When the first tissue is positioned between the first emitter and the first detector, light emitted from the first emitter passes through the first tissue and is received by the first detector which is configured to generate a second signal in response to the presence of the first tissue.

In one or more implementations of the fixation device, the second signal may be indicative of a thickness of the first tissue. In one or more implementations of the fixation device, the second signal is indicative of a tissue type.

In one or more implementations of the fixation device, the first detector or the first emitter may be coupled to the first proximal element at a location that may be 50% of the length of the first proximal element.

In one or more implementations of the fixation device, the first detector or the first emitter may be coupled to the first proximal element at a location that may be between the free end of the first proximal element and 50% of the length of the first proximal element.

In one or more implementations of the fixation device, the first emitter may include a light source that may be one of a light-emitting diode (LED) and a laser diode. In some examples, the first emitter may be configured to emit light with a wavelength selected from the range of 1,000 to 3,000 nm. In other examples, the first emitter may be configured to emit light with a wavelength selected from the range of 700 to 1,500 nm.

In one or more implementations of the fixation device, the first detector may include a detector component that includes one of a photodiode, phototransistor, charge-coupled device (CCD), complementary metal-oxide-semiconductor (CMOS), and photomultiplier tube (PMT).

In a further aspect of the present disclosure, an interventional system includes a delivery system that has a delivery handle and a delivery catheter extending from the delivery device handle. The system also includes a fixation device that has a first clamp and a second clamp that each define an open end and a closed end of the respective first and second clamps. The system further includes an optical sensing system that has a signal processing unit, a first optical sensor, and an interface communicatively coupling the first optical sensor with the signal processing unit. The first optical sensor has a first emitter and a first detector. The first emitter is configured to generate light in an infrared (IR) or near infrared (NIR) spectrum, and the first detector is configured to receive light and generate a signal indicative of one or more characteristics of a first tissue within the first clamp.

In one or more implementations of the system, the first emitter includes a light source. In some example, the light source is one of a light-emitting diode (LED) and a laser diode. In other examples, the first emitter is configured to emit light with a wavelength selected from the range of 1,000 to 3,000 nm. In further example, the first emitter is configured to emit light with a wavelength selected from the range of 700 to 1,500 nm.

In one or more implementations of the system, the one or more characteristics may include a distance between the first optical sensor and the first tissue. In one or more implementations of the system, the one or more characteristics may include tissue type.

In one or more implementations of the system, the first detector may include a detector component that includes one of a photodiode, phototransistor, charge-coupled device (CCD), complementary metal-oxide-semiconductor (CMOS), and photomultiplier tube (PMT).

In one or more implementations of the system, the signal processing unit may be coupled to the delivery handle. In some examples, the signal processing unit may include a power source configured to drive the first emitter and the first detector. Further, in some examples, the control unit may further include an output device. For example, the output device may be disposed within the delivery device handle. Additionally, the output device may be one of a screen, monitor, television, and touchscreen.

In one or more implementations of the system, the interface may be a hardwired interface extending through the delivery catheter. The delivery catheter may include a shaft with a plurality of spring arms. The spring arms may each have first electrical terminal and an actuator rod extendable through the shaft. Additionally, the center portion of the fixation device may include a coupling member with second electrical terminals corresponding with the first electrical terminals. In some examples, moving the actuator rod in a first direction may urge the spring arms into engagement with the coupling member and the first electrical terminals into engagement with the second electrical terminals. Also, moving the actuator rod in a second direction may disengage the spring arms from the coupling member and the first electrical terminals from the second electrical terminals.

In one or more implementations of the system, the interface may be a wireless interface enabled with a wireless protocol. The wireless protocol may be one of Bluetooth Low Energy (BLE), Near Field Communication (NFC), and Zigbee.

In one or more implementations of the system, the signal processing unit may further include an emitter optical fiber coupled to the first emitter and a detector optical fiber coupled to the first detector. The emitter optical fiber and the detector optical fiber may extend through a lumen of the delivery catheter. In some examples, the emitter optical fiber and the detector optical fiber may each extend into the fixation device and may be coupled thereto. In this regard, in one example, the emitter optical fiber and the detector optical fiber may be disposed within the closed end of the first clamp. In another example, the detector optical fiber may be coupled to the first proximal element, and the emitter optical fiber may be coupled to the first distal element such that it is directed toward the detector optical fiber and so that light emitted from the emitter optical is received in the detector optical fiber. In a further example, the detector optical fiber may be coupled to the first distal element, and the emitter optical fiber may be coupled to the first proximal element such that it is directed toward the detector optical fiber and so that light emitted from the emitter optical is received in the detector optical fiber. In other examples, the optical fiber and the detector optical fiber terminate at an end of the delivery catheter such that they are directed towards the fixation device and so that light emitted from the emitter optical fiber intersects tissue within the first clamp, and light reflected from the tissue is received by the detector optical fiber.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1A is a schematic view of an interventional system for repairing a heart valve.

FIG. 1B is a schematic view of a fixation device according to an implementation of the present disclosure grasping leaflets of a mitral valve.

FIG. 1C is a schematic view of an exemplary deployment sequence of a fixation device illustrated in a series of stages.

FIG. 2A is a cross-sectional representation of a heart illustrating its four valves.

FIG. 2B is a cross-sectional representation of a heart illustrating the left ventricle and left atrium during systole.

FIG. 2C is a schematic view of a mitral valve during normal coaptation.

FIG. 2D is a schematic view of a mitral valve during regurgitant coaptation.

FIG. 2E is an atrial-side view of a fixation device according to an implementation of the present disclosure in a desired orientation in relation to adjacent leaflets of a mitral valve.

FIG. 3A is an enlarged view of a fixation device according to an implementation of the present disclosure in an open or insertion configuration.

FIG. 3B is an enlarged view of the fixation device of FIG. 3A illustrating a sensor assembly for detecting leaflet insertion.

FIG. 4A is a schematic view of the sensor assembly with first and second signal emitters forming a combined sensing area.

FIGS. 4B-4G are schematic views of a fixation device and sensor assembly illustrating detection of leaflet insertion during a transcatheter edge-to-edge repair procedure.

FIG. 4H is a schematic view of a controller determining an insertion characteristic and present a representation via a feedback indicator.

FIGS. 4I and 4J are schematic pinch force diagrams along proximal and distal elements of different lengths.

FIG. 4K is a schematic view of a optical sensing system according to an embodiment of the present disclosure.

FIG. 4L is a schematic view of a distal end of an interventional system according to an embodiment of the present disclosure and including an exemplary fixation device having one or more optical sensors of the system of FIG. 4K coupled thereto.

FIGS. 4M and 4N are partial schematic views of an optical sensor configuration according to an example of the interventional system of FIG. 4L.

FIG. 4O is a partial schematic view of an optical sensor configuration according to another example of the interventional system of FIG. 4L.

FIG. 4P is a partial schematic view of an optical sensor configuration according to a further example of the interventional system of FIG. 4L.

FIG. 4Q is a schematic view of a distal end of an interventional system according to another embodiment of the present disclosure and including an exemplary fixation device having one or more optical sensors of the system of FIG. 4K coupled thereto.

FIG. 4R is a bottom view of a proximal element according to an example of the fixation device of FIG. 4Q.

FIG. 4S is a bottom view of a proximal element according to another example of the fixation device of FIG. 4Q.

FIG. 4T is a perspective view of a distal element according to an example of the fixation device of FIG. 4Q.

FIG. 4U is a perspective view of a proximal element according to a further example of the fixation device of FIG. 4Q.

FIG. 4V is a perspective view of an interventional system according to an embodiment of the present disclosure.

FIG. 4W is a perspective view of gripper element lines according to an example of the interventional system of FIG. 4V.

FIG. 4X is a schematic view of an optical sensing system according to another embodiment of the present disclosure.

FIG. 4Y is a schematic view of a distal end of an interventional system according to a further embodiment of the present disclosure and including an exemplary fixation device having one or more optical sensors of the system of FIG. 4X coupled thereto.

FIG. 4Z is a schematic view of a distal end of an interventional system according to another embodiment of the present disclosure and including an exemplary fixation device having one or more optical sensors of the system of FIG. 4X coupled thereto.

FIG. 4AA and 4AB are schematic views of a distal end of an interventional system according to a further embodiment of the present disclosure and including an exemplary fixation device having one or more optical sensors of the system of FIG. 4X coupled thereto.

FIG. 4AC is a schematic end view of a catheter of the interventional system of FIG. 4AA and 4AB.

FIG. 4AD is a front elevational view of a distal end of an interventional system according to an embodiment of the present disclosure and including a ring and a fixation device coupled to a distal end of a delivery catheter shaft.

FIG. 4AE is a perspective view of the ring of FIG. 4AD with a partial delivery catheter shaft extending therethrough.

FIG. 4AF is a bottom view of the ring of FIG. 4AD.

FIG. 4AG is a bottom perspective view of a ring and sensor packages coupled thereto according to another embodiment of the present disclosure and also showing a partial delivery catheter shaft extending through the ring.

FIG. 4AH is a front elevational view of the ring and sensor packages of FIG. 4AG with a partial delivery catheter shaft extending therethrough.

FIG. 4AI is a bottom view of the ring and sensor packages of FIG. 4AG illustrating a relationship of emitter-detector pairs relative to a gripping device having first and second proximal elements.

FIG. 4AJ is a bottom perspective view of a ring and a sensor package coupled thereto according to a further embodiment of the present disclosure.

FIG. 4AK is a front elevational view of the sensor package of FIG. 4AJ.

FIG. 4AL is a bottom view of the ring and sensor package of FIG. 4AJ illustrating a relationship of emitter-detector pairs relative to a gripping device having first and second proximal elements.

FIG. 5 is a perspective view of a fixation device according to another implementation of the present disclosure.

FIG. 6A is a perspective view of a gripping device of the fixation device of FIG. 5 according to an implementation of the present disclosure.

FIG. 6B is an elevational view of the gripping device of FIG. 6A.

FIG. 6C is a perspective view of a gripping device according to another implementation of the present disclosure.

FIG. 6D is a partial schematic view of the gripping device of FIG. 6C coupled to a distal element of the fixation device of FIG. 5.

FIG. 6E is a partial schematic view of a gripping device according to an alternative implementation of the present disclosure coupled to a distal element according to an alternative implementation of the present disclosure,

FIG. 7 illustrates the released fixation device of FIG. 5 in an example of a closed position.

FIG. 8A is an elevational view of a coupling system according to an implementation of the present disclosure for coupling the fixation device of FIG. 5 and a delivery system.

FIGS. 8B and 8C are schematic views of the coupling system of FIG. 8A in respective first and second configurations.

FIGS. 8D and 8E are schematic cross-sectional views of a coupling system according to another implementation of the present disclosure for coupling a fixation device, such as the fixation device of FIG. 5, and a delivery system.

FIGS. 8F and 8G depict the fixation device of FIG. 5 in an example of a closed position or delivery position.

FIGS. 8H and 8I depict the fixation device of FIG. 5 in an example of an open position.

FIGS. 8J and 8K illustrate the fixation device of FIG. 5 in an example of a capture position.

FIGS. 8L and 8M depict an example of an inverted position of the fixation device of FIG. 5.

FIG. 8N illustrates the fixation device of FIG. 5 in the closed position wherein the leaflets are captured and coapted.

FIG. 8O illustrates the fixation device of FIG. 5 released from the shaft of the delivery system while in the closed position.

FIG. 8P is a perspective view of the fixation device of FIG. 5 including a lock according to an implementation of the present disclosure and illustrating a plurality of proximal element lines and a lock line coupled to the fixation device.

FIG. 8Q is an elevational view of the lock and proximal elements of the fixation device of FIG. 8P and illustrating a lock line and single proximal element line respectively coupled thereto.

FIG. 8R is a schematic view of the fixation device of FIG. 5 coupled to a delivery system and illustrating a plurality of proximal element lines coupled to a shaft of the delivery system.

FIG. 8S is a perspective view of the fixation device of FIG. 5 including a covering.

FIGS. 9A and 9B are partial enlarged views of a distal end portion of the delivery system shaft of FIG. 8R according to an implementation of the present disclosure.

FIG. 9C is a cross-sectional view of the delivery system shaft taken along line C-C of FIG. 9B.

FIG. 9D is a partial perspective view of a distal end portion of one of the proximal element lines of FIG. 8R including a catch element according to an implementation of the present disclosure.

FIG. 9E is a partial elevational view of the delivery system shaft of FIG. 9A having holes configured to receive the catch element of FIG. 9D.

FIG. 9F is a partial elevational view of the delivery system shaft of FIG. 9A and an actuator rod disposed therein intersecting the holes of the delivery system shaft.

FIG. 9G is a partial elevational view of a distal end portion of one of the proximal element lines of FIG. 8R including a catch element according to another implementation of the present disclosure.

FIG. 10A is an enlarged cross-sectional view of the lock of FIG. 8P taken along a midline thereof and in an unlocked configuration.

FIG. 10B is an enlarged elevational view of the lock of FIG. 8P and in a locked configuration.

FIG. 10C is a perspective view of a release harness of the lock of FIG. 8P.

FIG. 10D is an elevational view of a lock of the fixation device of FIG. 5 according to another implementation of the present disclosure.

FIG. 10E is a transparent perspective view of a binding plate of the lock of FIG. 10D.

FIG. 10F is an enlarged elevational view of the lock of FIG. 10D.

FIG. 11 is a partial perspective view of an interventional system according to an implementation of the present disclosure and including an exemplary delivery system, an exemplary steerable guide system, and an exemplary stabilizer.

FIG. 12A is a partial perspective view of the delivery system of FIG. 11.

FIG. 12B is a partial cutaway view of the delivery system of FIG. 11 and the fixation device of FIG. 5 coupled thereto.

FIG. 12C is a cross-sectional view of a positioner assembly of the delivery system of FIG. 11.

FIG. 13A is a partial perspective view of the positioner assembly of FIG. 12C including a rotation control system according to an implementation of the present disclosure and in an example of a first configuration.

FIGS. 13B and 13C are partial perspective views of the positioner assembly and rotation control system of FIG. 13A with the rotation control system in an example of a second configuration.

FIG. 14A is a partial perspective view of a gripper control assembly and a fluid management assembly of the delivery system of FIG. 11 and each according to an implementation of the present disclosure.

FIG. 14B is a partial elevational view of the gripper control assembly of FIG. 14A.

FIG. 14C is a partial cross-sectional view of the gripper control assembly of FIG. 14A taken along a midline thereof.

FIG. 15A is a partial transparent perspective view of a lock control assembly of the delivery system of FIG. 11 according to an implementation of the present disclosure.

FIG. 15B is another partial transparent perspective view of the lock control assembly of FIG. 15A.

FIG. 15C is an exploded view of the lock control assembly of FIG. 15A.

FIGS. 15D and 15E are perspective views of a lock handle shaft of the lock control assembly of FIG. 15A in an example of an unlocked configuration.

FIGS. 15F and 15G are perspective views of a lock handle shaft of the lock control assembly of FIG. 15A in an example of a locked configuration.

FIG. 15H is a perspective view of a post of lever of lock handle shaft of the lock control assembly of FIG. 15A.

FIG. 15I is a partial transparent perspective view of the lock control assembly of FIG. 15A with a lock knob thereof in an example of a lock configuration.

FIG. 15J is a partial transparent perspective view of the lock control assembly of FIG. 15A with a lock knob thereof in an example of an unlocked configuration.

FIG. 15K is a partial transparent perspective view of the lock control assembly of FIG. 15A with a lock knob thereof in an example of a third configuration.

FIGS. 15L and 15M are partial transparent perspective views of the lock control assembly of FIG. 15A with a release slider thereof in an example of a release position.

FIG. 15N is a partial transparent perspective view of the lock control assembly of FIG. 15A including lock knob thereof being removed.

FIG. 15O is a perspective view of the lock control assembly of FIG. 15A partially disassembled.

FIG. 16A is a cross-sectional view of a delivery catheter fastening assembly of the delivery system of FIG. 11 according to an implementation of the present disclosure.

FIG. 16B illustrates the delivery catheter fastening system of FIG. 16A being rotated in first and second directions.

FIGS. 17A and 17B illustrate the delivery system of FIG. 11 translating between first and second positions along a shaft of the delivery catheter fastening assembly of FIG. 16A.

FIG. 17C is a partial elevational view of the interventional system of FIG. 11 including a proximal attachment assembly thereof.

FIG. 17D is a perspective view of the proximal attachment assembly of FIG. 17C.

FIGS. 17E and 17F are elevational views of the proximal attachment assembly of FIG. 17D in an example of a first configuration and an example of a second configuration, respectively.

FIGS. 17G and 17H are elevational views of the proximal attachment assembly of FIG. 17D in the first configuration and the second configuration, respectively.

FIG. 18A is partial perspective view of the interventional system of FIG. 11 including an exemplary distal attachment assembly thereof.

FIG. 18B illustrates the distal attachment connected to an outer guide catheter handle of the interventional system shown in cutaway.

FIG. 18C is a partial cross-sectional view of the distal attachment assembly of FIG. 18B taken along a midline thereof.

FIG. 19A is a perspective view of the stabilizer of FIG. 11.

FIG. 19B is a partial perspective view of a distal attachment of the stabilizer of FIG. 19A.

FIG. 19C is a partial perspective view of a proximal attachment of the stabilizer of FIG. 19A.

FIGS. 19D and 19E illustrate the distal attachment assembly of FIG. 18B being attached to the distal attachment FIG. 19A.

FIGS. 19F and 19G illustrate the proximal attachment assembly of FIG. 18B attached to the proximal attachment of FIG. 19A.

FIGS. 19H and 19I illustrate the proximal attachment assembly of FIG. 17D being moved relative to the proximal attachment of the stabilizer of FIG. 19A.

FIGS. 19J and 19K illustrate an example of manipulation of the fixation device during a procedure using the interventional system and stabilizer of FIG. 19A.

FIG. 20 is a flowchart illustrating an example arrangement of operations for a method of detecting leaflet insertion during a transcatheter edge-to-edge repair procedure.

FIG. 21 is a schematic view of an example computing device that may be used to implement the systems and methods described herein.

Like reference symbols in the various drawings indicate like elements.

### DETAILED DESCRIPTION

Transcatheter edge-to-edge repair (TEER) has emerged as a treatment option for patients with mitral valve regurgitation and tricuspid valve regurgitation. TEER procedures may involve the percutaneous delivery of a fixation device to a target heart valve, where the fixation device is positioned to grasp adjacent valve leaflets and hold them together to improve coaptation and reduce regurgitant flow. These procedures may be performed using catheter-based delivery systems that navigate through the vasculature to reach the heart, allowing treatment without the need for open-heart surgery.

During a TEER procedure, a clinician may position the fixation device at the target valve and attempt to capture the valve leaflets between fixation elements of the device. Proper leaflet insertion into the fixation device may be important for achieving adequate grasping and, consequently, for reducing regurgitation. In some cases, insufficient leaflet insertion may result in suboptimal grasping, which may lead to inadequate reduction of regurgitation, the need for repositioning of the fixation device, or other procedural complications. Conversely, excessive leaflet insertion may in some instances cause unintended tissue trauma or interference with valve function.

Clinicians performing TEER procedures may rely on various imaging modalities to visualize the fixation device and the valve leaflets during the procedure.

Fluoroscopy may provide real-time visualization of radiopaque components of the delivery system and fixation device, but may offer limited information regarding the position of soft tissue structures such as the valve leaflets. Echocardiography, including transesophageal echocardiography, may provide visualization of both the fixation device and the valve leaflets, but image quality may vary depending on patient anatomy, acoustic windows, and other factors. In some cases, precisely determining the position of a leaflet relative to the fixation elements of the device using these imaging modalities alone may be challenging.

The challenge of confirming adequate leaflet insertion may be compounded by the dynamic nature of the cardiac environment. The heart continues to beat during the procedure, causing the valve leaflets to move with each cardiac cycle. This motion may make it difficult to assess whether a leaflet is properly positioned within the graspable space of the fixation device at any given moment. Additionally, blood flow and the presence of blood in the imaging field may further obscure visualization of the leaflet position relative to the fixation device components.

Accordingly, implementations herein are directed to an interventional system for repairing a heart valve. In some implementations, the interventional system may include a delivery catheter having a proximal end portion and a distal end portion, and a fixation device releasably coupled to the distal end portion of the delivery catheter. The fixation device may include a distal fixation element having an engagement surface and being actuatable between an open position and a closed position, and a proximal fixation element having an engagement surface and being actuatable between an open position and a closed position. In the open position, a graspable volume of space may be defined between the engagement surface of the proximal fixation element and the engagement surface of the distal fixation element for receiving a leaflet of the heart valve. One or more reflective markers may be disposed on the engagement surface of the distal fixation element. A sensor assembly may be disposed on the distal end portion of the delivery catheter and may include a signal emitter configured to direct an emitted signal along an emission path toward the one or more reflective markers and a signal receiver configured to receive a reflected signal of the emitted signal off of the one or more reflective markers. As a leaflet is inserted into the graspable volume of space, the leaflet may progressively occlude the reflective markers, resulting in a corresponding change in the reflected signal that indicates the depth of leaflet insertion.

In some implementations, the interventional system may detect leaflet insertion by sensing reflections directly off of the leaflet tissue rather than by sensing occlusion of reflective markers. In such implementations, the sensor assembly may include a signal emitter arranged to direct an emitted signal along an emission path toward the graspable volume of space defined by the proximal fixation element and the distal fixation element when in the open position, and a signal receiver arranged to receive a reflected signal of the emitted signal off of the leaflet of the heart valve when positioned within the graspable volume of space. As the leaflet moves into the graspable volume of space, the intensity of the reflected signal may change in proportion to the position of the leaflet, thereby providing an indication of leaflet insertion. These approaches may address the challenge of confirming adequate leaflet insertion by providing real-time feedback to a clinician regarding the position of a leaflet relative to the fixation device during the grasping process, which may reduce the need for repositioning and improve procedural outcomes.

Implementations herein are further directed to a method of detecting leaflet insertion during a TEER repair procedure. In some implementations, the method may include positioning a fixation device at a leaflet of a heart valve within a heart, where the fixation device includes a distal fixation element having an engagement surface and a proximal fixation element having an engagement surface, each actuatable between an open position and a closed position, The method may include actuating the distal fixation element and the proximal fixation element to the open position and emitting a signal from a sensor assembly toward the engagement surface of the distal fixation element. The method may further include receiving, by the sensor assembly, signal data corresponding to a reflected signal, and determining an insertion characteristic of the leaflet relative to the distal fixation element based on the signal data. A representation of the insertion characteristic may be presented by a feedback indicator, enabling a clinician to assess leaflet position in real-time before completing the grasp. The method may then include actuating the proximal fixation element toward the closed position to grasp the leaflet.

This approach may enable clinicians to confirm adequate leaflet insertion prior to grasping, potentially reducing the need for repositioning and improving procedural efficiency.

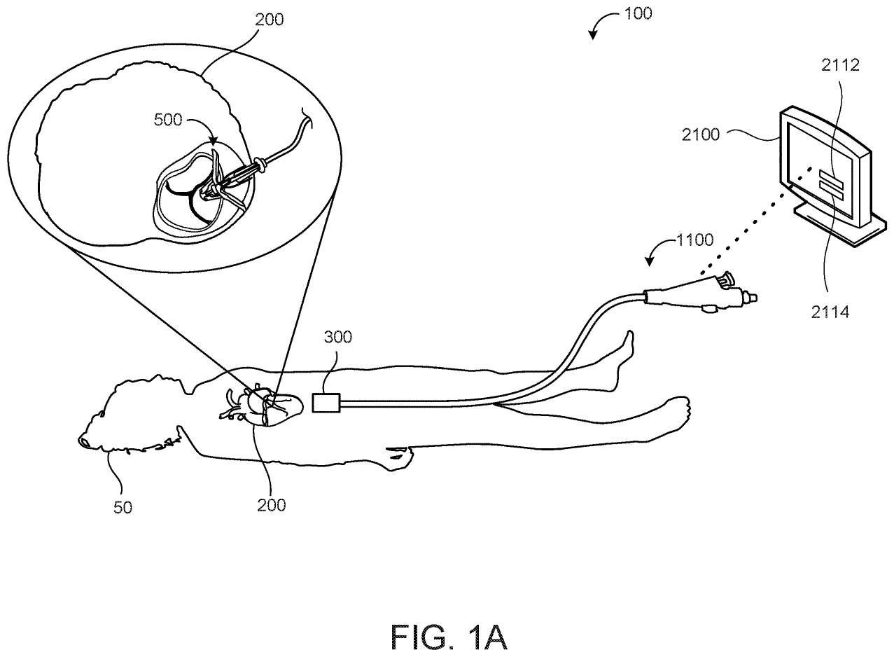

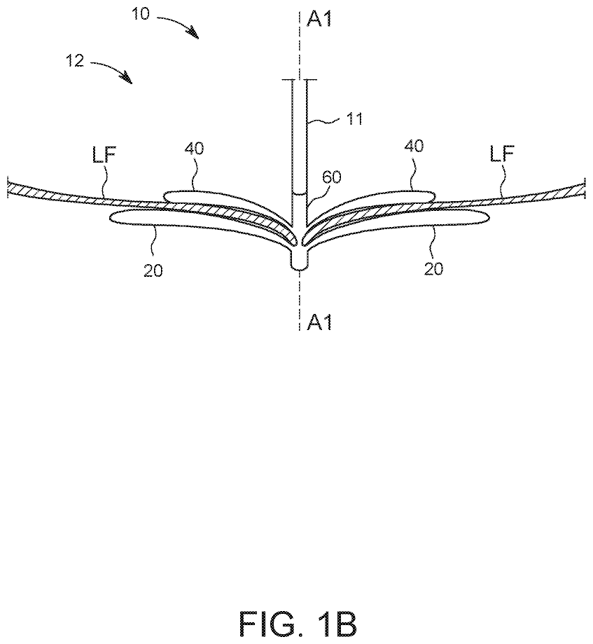

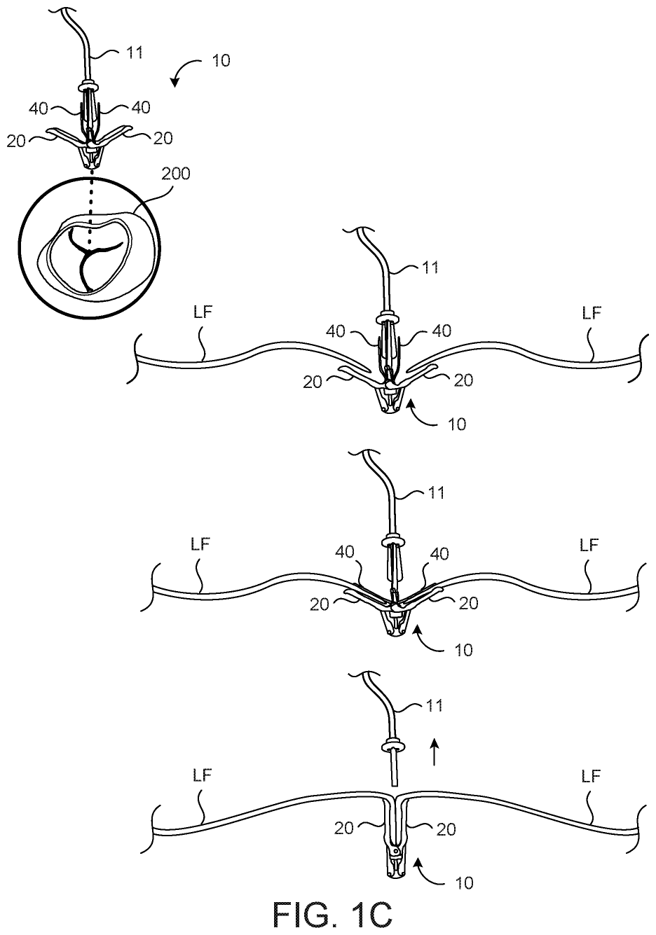

Referring to FIG. 1A, in some implementations, a system 100 includes an interventional system 1100 for repairing a heart valve of a patient 50. The interventional system 1100 includes a sensor assembly 400, a fixation device 500. A steerable guide system 1105 and a computing device 2100 may also form part of the interventional system 1100. The steerable guide system 1105 is generally configured to navigate the fixation device 500 percutaneously through the patient 50 to a target treatment site, such as a heart valve of a heart 200. The computing device 2100 may include data processing hardware 2112 in communication with memory hardware 2114. Together, the components of the interventional system 1100 are configured to repair a heart valve of the heart 200, such as by performing a transcatheter edge-to-edge repair (TEER) procedure.

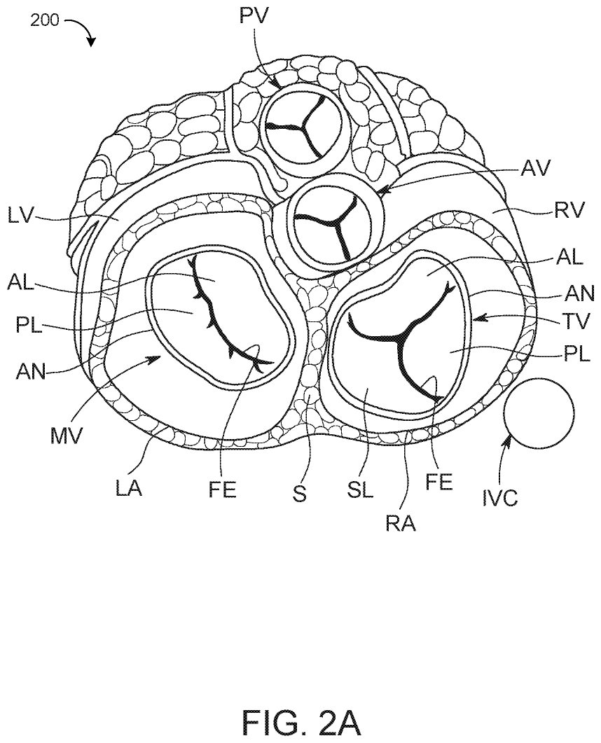

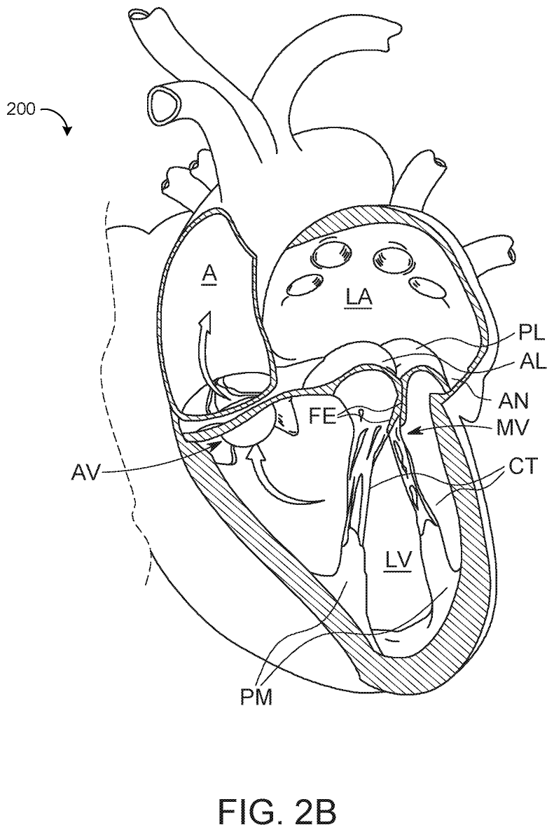

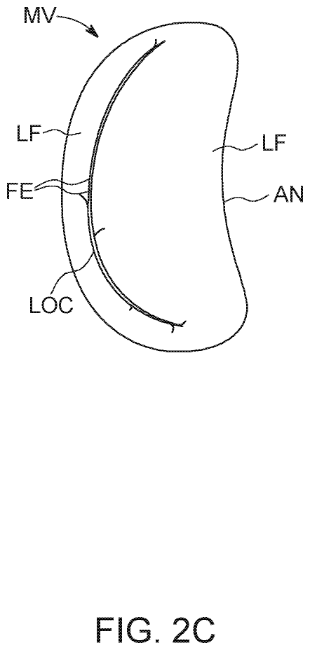

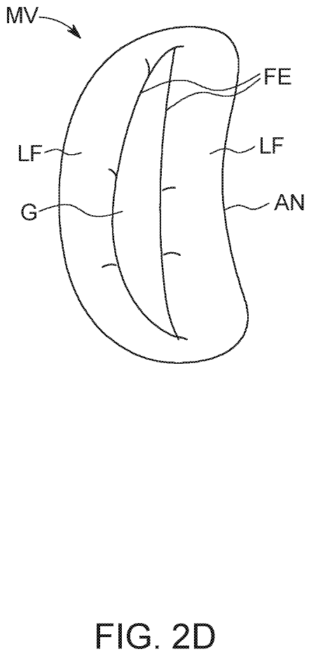

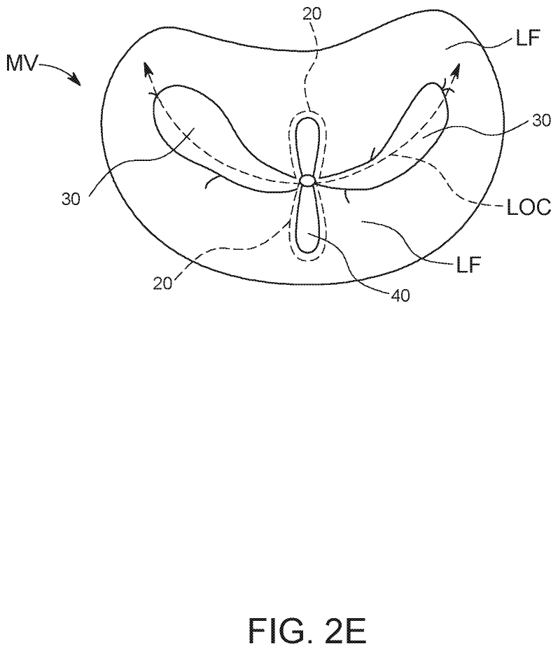

The valves of the normal heart 200 are illustrated in FIGS. 2A and 2B. These valves include a mitral valve MV, a tricuspid valve TV, an aortic valve AV, and a pulmonary valve PV. The mitral valve MV separates a left atrium LA and a left ventricle LV, and the tricuspid valve TV separates a right atrium RA and a right ventricle RV. The mitral valve MV and the tricuspid valve TV are sometimes referred to as the atrioventricular valves. The mitral valve MV is a bicuspid valve in that it has two leaflets referred to as a posterior leaflet PL and an anterior leaflet AL. The tricuspid valve TV typically has three leaflets referred to as the anterior leaflet AL, the posterior leaflet PL, and a septal leaflet SL. However, studies have shown that although the tricuspid valve TV is typically composed of three leaflets of unequal size, in many cases two or more than three leaflets may be present as anatomic variants in healthy subjects. Thus, reference herein to the tricuspid valve TV should be understood to refer to the atrioventricular valve located between the right atrium RA and the right ventricle RV regardless of the number of leaflets be it two, three, or more than three leaflets. However, exemplary implementations discussed herein refer to the usual anatomic structure of the tricuspid valve TV that includes three leaflets.

As illustrated in FIG. 2A, the anterior leaflet AL and the posterior leaflet PL of the mitral valve MV extend from a valve annulus AN to respective free edges FE. The free edges FE are secured to the lower portions of the left ventricle LV through chordae tendineae CT (referred to hereinafter as the chordae CT). The chordae CT includes a plurality of branching tendons that are attached to papillary muscles PM at the lower portions of the left ventricle LV and extend upwardly to the lower surfaces of each of the valve leaflets where they are attached. The three leaflets of the tricuspid valve TV similarly extend from the valve annulus AN to respective free edges FE which are secured via the chordae CT to the papillary muscles PM of the right ventricle RV.

The mitral valve MV depicted in FIGS. 2A and 2B illustrate the proper functioning of an atrioventricular valve during ventricular systole. As the ventricles contract, the free edges FE of adjacent leaflets LF meet along a line of coaptation LOC. The joinder of the leaflets LF at this line of coaptation LOC seals off the ventricle from the atrium and prevents the back flow of blood or “regurgitation” from entering into the atrium. Thus, with the right atrium RA and the left atrium LA respectively sealed off by the mitral valve MV and the tricuspid valve TV, blood in the left ventricle LV can only flow through the aortic valve AV to the body, and blood in the right ventricle RV can only flow through the pulmonary valve PV to the lungs.

A number of structural defects in the heart 200 can cause mitral valve regurgitation (“MVR”) and/or tricuspid valve regurgitation (“TVR”). MVR and TVR occur when their respective leaflets LF do not close properly allowing leakage from the ventricle into the atrium. The mitral valve MV depicted in FIG. 2C illustrates valvular insufficiency of an atrioventricular valve resulting in regurgitation. In the depicted example, an enlargement of the heart 200 may cause the valve annulus AN to become enlarged, making it impossible for the free edges FE of the valve leaflets LF to meet during systole. This may result in a gap G between the leaflets LF which allows blood to leak through the valve. In another example, ruptured or elongated chordae CT can cause a valve leaflet LF to prolapse at least due to inadequate tension transmitted to the leaflet LF via the chordae CT. While an adjacent leaflet LF may maintain a normal profile, the prolapsing leaflets LF may flail about preventing the proper joinder between the leaflets LF resulting in leakage into the atrium. In a further example, regurgitation can occur in patients who have suffered ischemic heart disease which may result in weak ventricular contractions insufficient to effect proper closure.

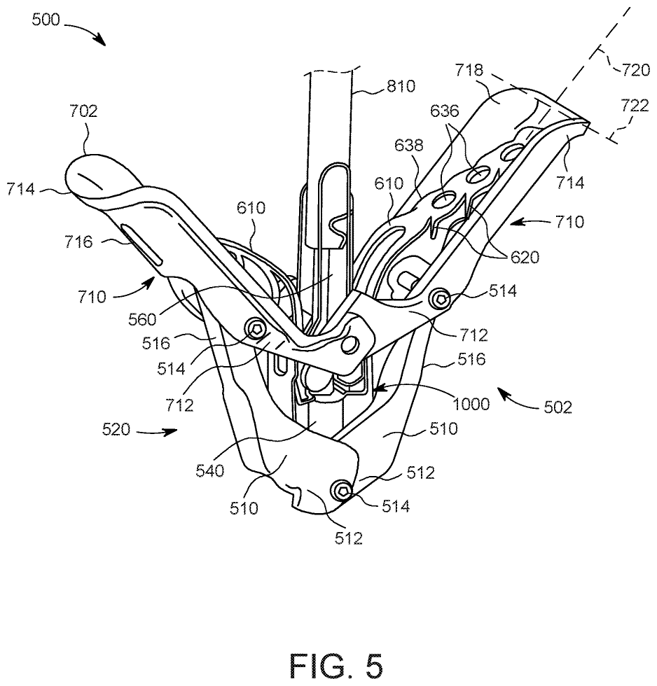

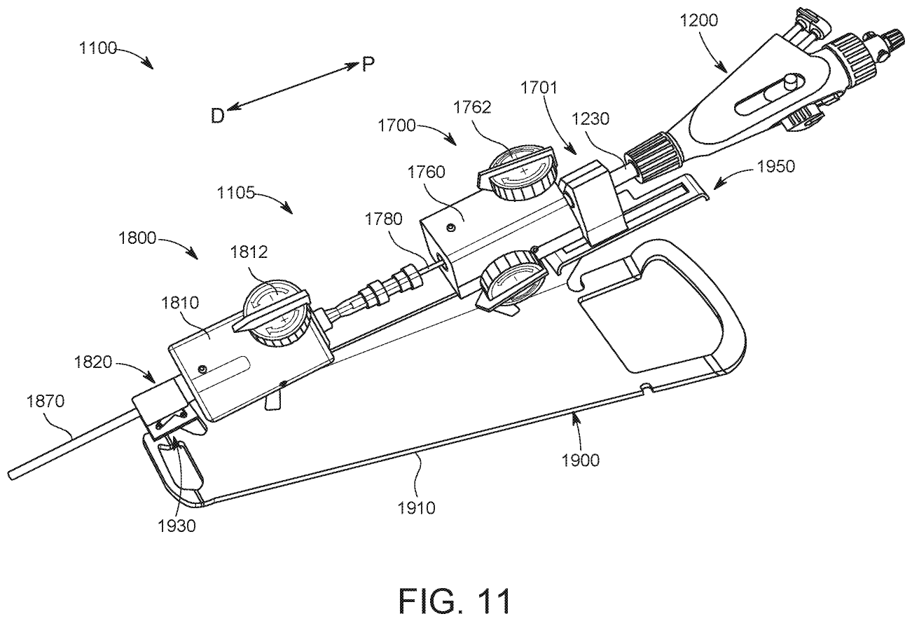

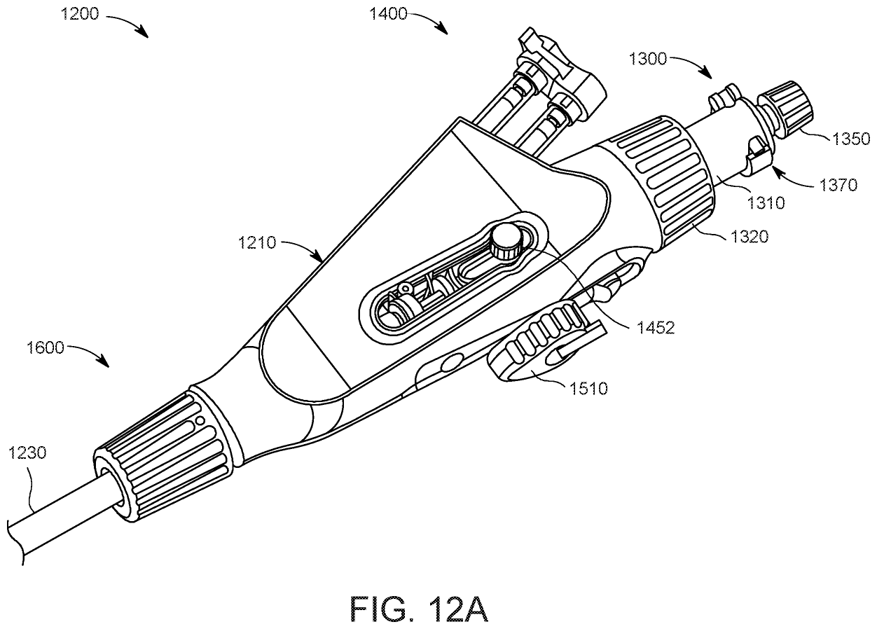

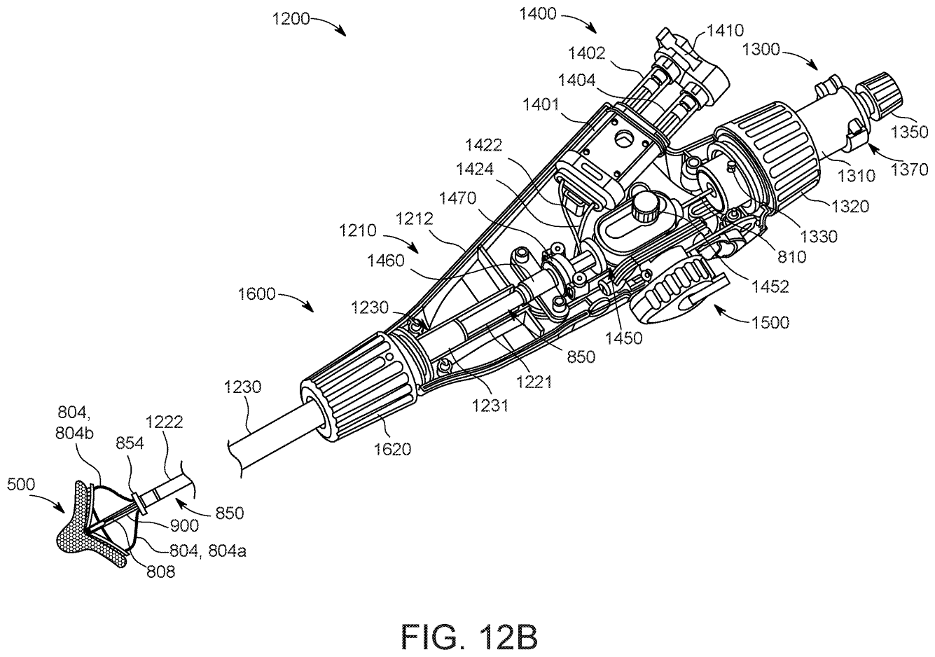

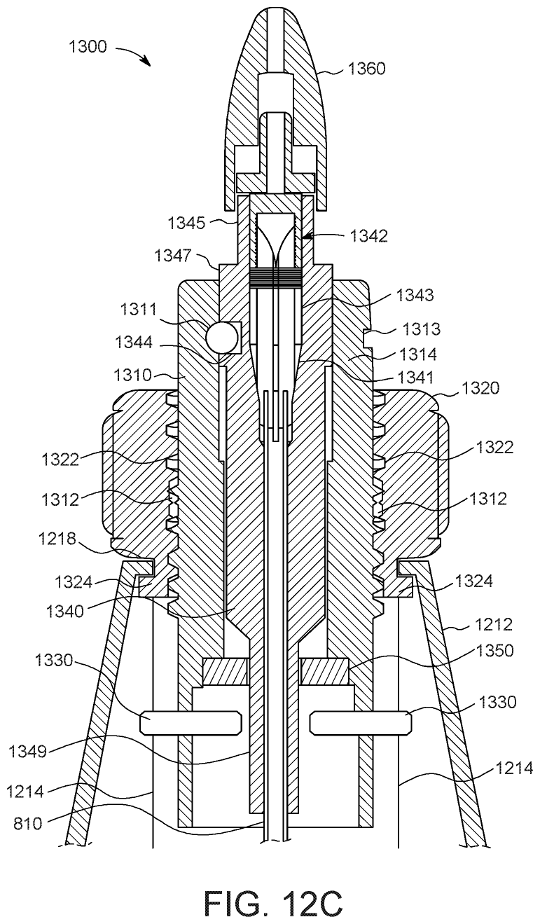

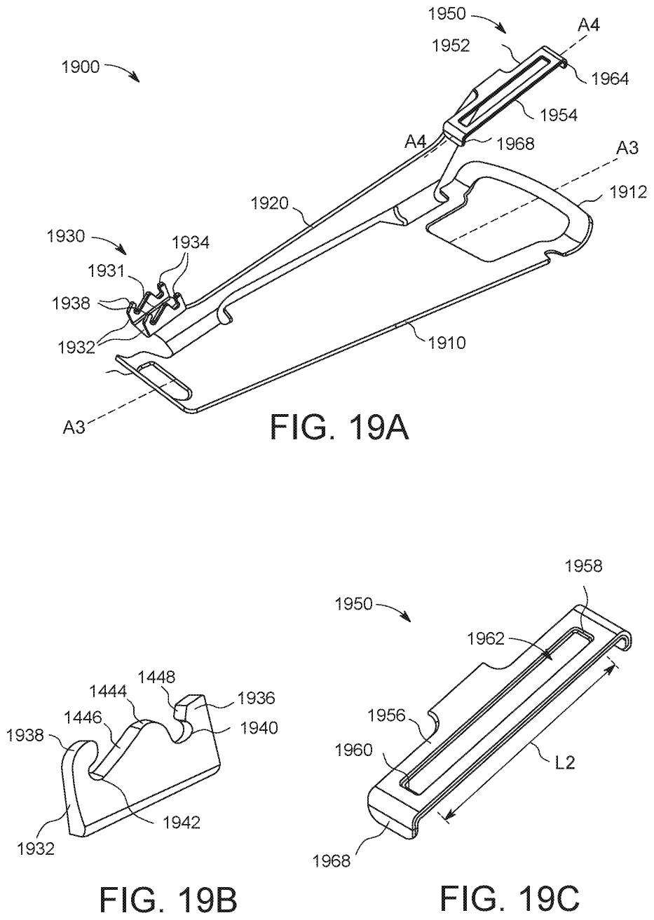

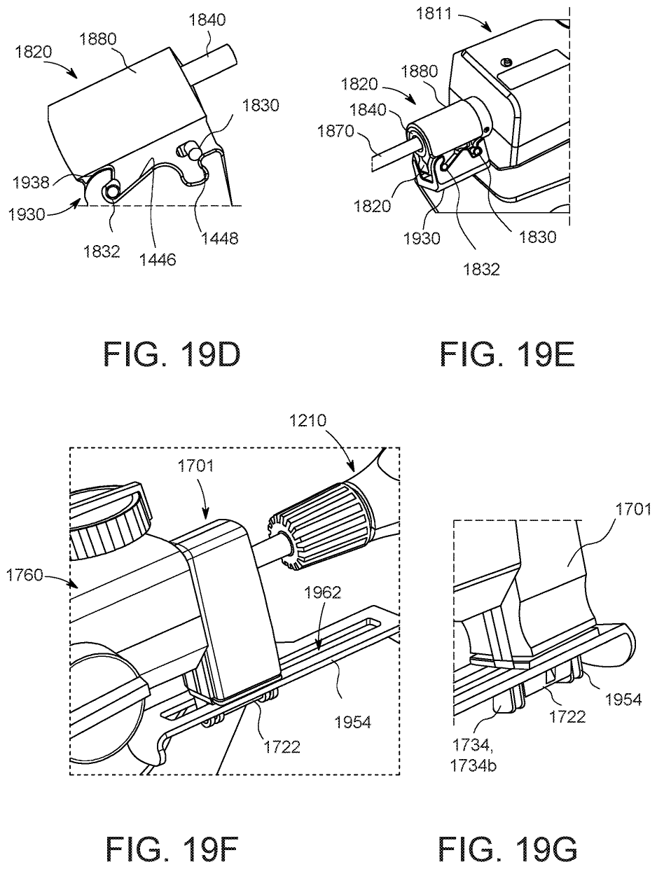

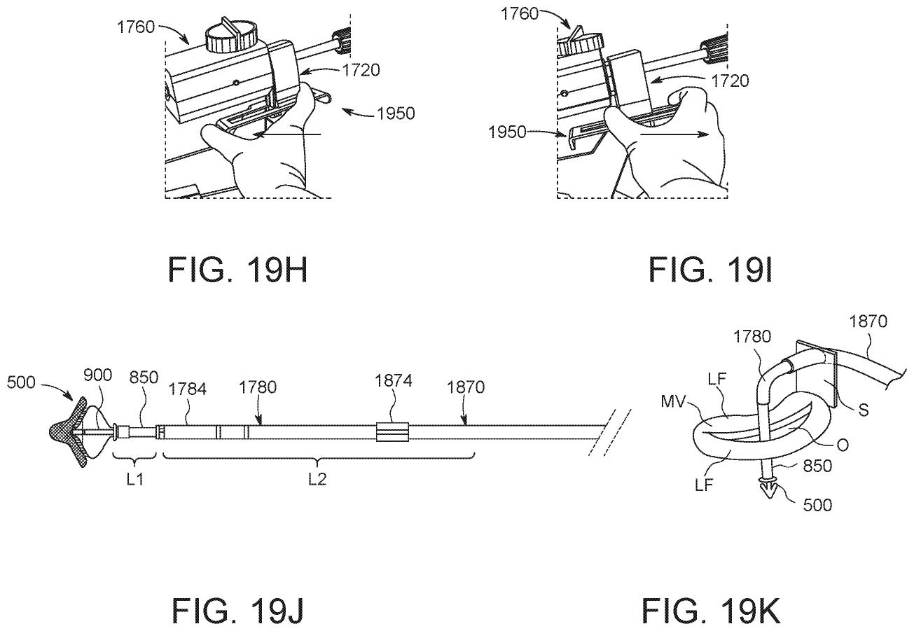

The present disclosure describes exemplary systems, devices, and methods for percutaneously repairing a valve to treat cardiac valve regurgitation, particularly MVR and TVR. For example, the interventional system 1100, according to an implementation of the present disclosure, may include the fixation device 500 (see e.g., FIG. 5), a delivery system 1200 (see e.g., FIG. 12B) for delivery to and deployment of the fixation device 500 within a heart valve. The steerable guide system 1105 (see e.g., FIG. 11) and a stabilizer 1900 ((see e.g., FIG. 19A) may also form part of the interventional system 1100. The steerable guide system 1105 provides a conduit to guide the delivery system 1200 and the fixation device 500 to the heart valve, while the stabilizer 1900 is configured for stabilizing and supporting the use of the delivery system 1200 and the steerable guide system 1105. The delivery system 1200 and the steerable guide system 1105 are collectively referred to herein as a multi-catheter system.

When referring to such disclosed systems, devices, and methods, the term “proximal” (P) shall mean closer to the user or in a direction toward a device to be manipulated by the user outside the patient's body (e.g., a delivery handle 1210 of the delivery system 1200), and the term “distal” (D) shall mean more distant from the user or in a direction toward a device that is positioned at the treatment site within the patient's body (e.g., the fixation device 500). With respect to the mitral valve MV and the tricuspid valve TV, “proximal” shall refer to the atrial or upstream side of the valve leaflets LF, and “distal” shall refer to the ventricular or downstream side of the valve leaflets LF.

FIG. 1B depicts a fixation device 12, according to an implementation of the present disclosure, grasping the leaflets LF of an atrioventricular valve, which is illustrated as the mitral valve MV. The fixation device 12 may be releasably coupled to a distal end of a shaft 11 of the delivery system 1200 to form an interventional tool 10. The fixation device 12 may include distal elements 20 (also referred to herein as distal fixation elements) and proximal elements 40 (also referred to herein as gripping elements). The distal elements 20 and the proximal elements 40 (also referred to herein as proximal fixation elements) may be moveable relative to each other and may protrude radially outward relative to a longitudinal axis A1 of the fixation device 12. As shown in FIG. 1B, the fixation device 12 may be positionable on opposite sides of adjacent leaflets LF of the valve so as to capture or retain the leaflets LF therebetween. In this regard, the proximal elements 40 may be positioned at a proximal side of the valve leaflets LF, and the distal elements 20 may be positioned on a distal side of the valve leaflets LF. The proximal elements 40 may be made from cobalt chromium, nitinol, or stainless steel, for example, and the distal elements 20 may be made from cobalt chromium or stainless steel, for example.

The fixation device 12 may be releasably coupled to the shaft 11 such that it can be detached and left behind as an implant to hold the leaflets LF together in the coapted position. In this regard, the fixation device 12 may be delivered to a target valve percutaneously using any one of a number of different approaches, such as via a transfemoral, a transapical, or a transjugular approach, for example. Thus, in some examples of treating MVR, the fixation device 12 may be delivered to the deficient mitral valve MV using a transfemoral approach in which the fixation device 12 is guided through an inferior vena cava IVC (see FIG. 2A), across an interatrial septum S, and into the left atrium LA where the fixation device 12 is advanced into the mitral valve MV.

Also, in some examples of treating TVR, the fixation device 12 may be guided transfemorally through the inferior vena cava IVC to the right atrium RA where the fixation device 12 is advanced to a desired position within the tricuspid valve TV.

FIG. 2E is an atrial-side view of the fixation device 12 in some examples of a desired orientation in relation to adjacent leaflets LF of an atrioventricular valve, such as the depicted mitral valve MV. The distal elements 20 and the proximal elements 40 are positioned to be substantially perpendicular to the line of coaptation LOC. Thus, in the case of the mitral valve MV, the fixation device 12 may be oriented perpendicular (+/−5 degrees) to the line of coaptation LOC between the posterior leaflet PL and the anterior leaflet AL, and in the case of the tricuspid valve TV, the fixation device 12 may be positioned perpendicular (+/−5 degrees) to a line of coaptation between the septal leaflet SL and the anterior leaflet AL, the septal leaflet SL and the posterior leaflet PL, or the anterior leaflet AL and the posterior leaflet PL, for example. The fixation device 12 may be moved roughly along the line of coaptation LOC to the location of regurgitation. The leaflets LF may be held in place so that, during diastole, the leaflets LF remain in position between the elements 20, 40 surrounded by openings 30 (also referred to herein as orifices) which result from the diastolic pressure gradient. Advantageously, the leaflets LF are coapted such that their proximal or upstream surfaces face each other in a vertical orientation, parallel to the direction of blood flow through the valve. The upstream surfaces may be brought together so as to be in contact with one another or may be held slightly apart but will preferably be maintained in the vertical orientation in which the upstream surfaces face each other at the point of coaptation. This simulates the double orifice geometry of a standard surgical bow-tie repair. Color Doppler echo will show if the regurgitation of the valve has been reduced. If the resulting flow pattern is satisfactory, the leaflets LF may be fixed together in this orientation. If the resulting color Doppler image shows insufficient improvement in valve regurgitation, the fixation device 500 may be repositioned. This may be repeated until an optimal result is produced wherein the leaflets LF are held in place.

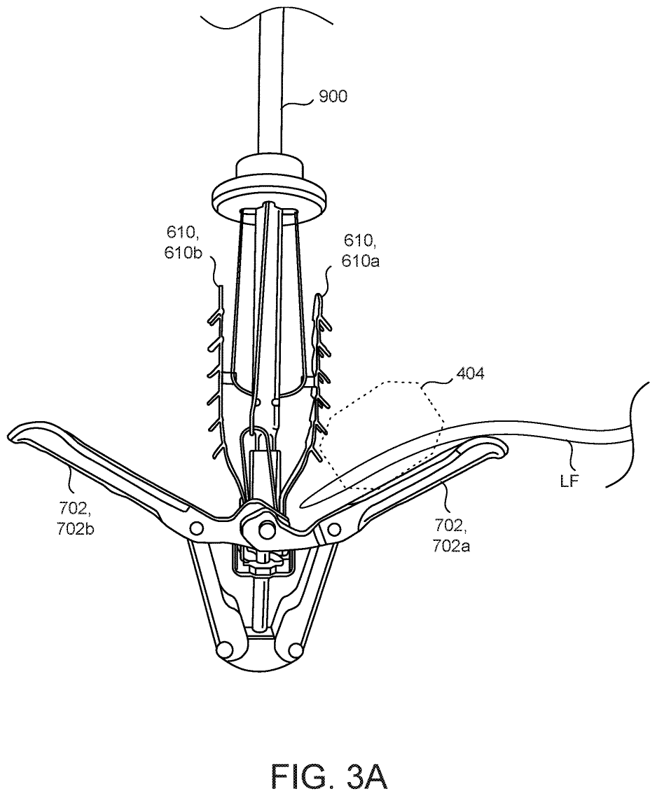

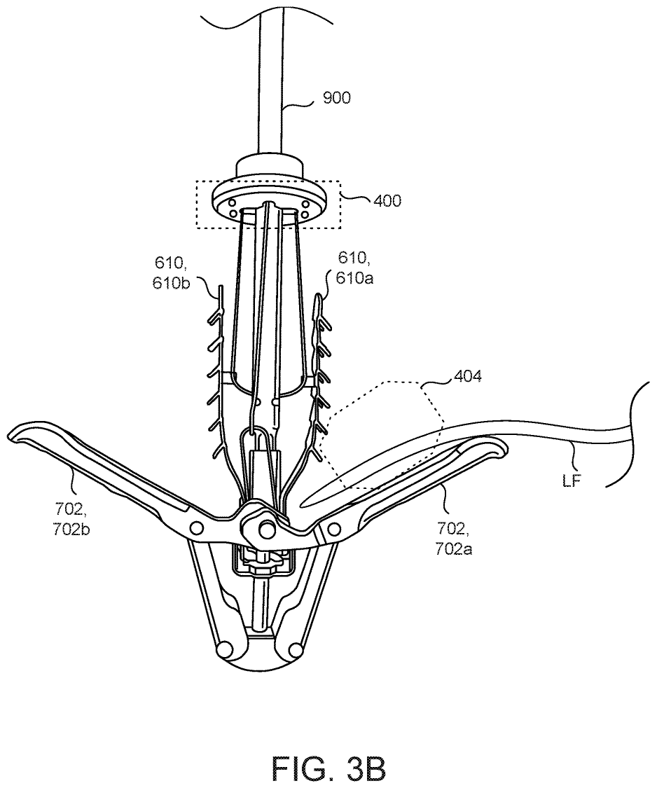

Referring now to FIG. 1C, an exemplary deployment sequence of the fixation device 12 is illustrated in a series of stages. In some implementations, the fixation device 12 includes one or more distal elements 20 (also referred to as distal fixation elements 20) and one or more proximal fixation elements 40. The fixation device 12 is releasably connected to the delivery system 1200 via a shaft 900. In a first stage (illustrated at the top of FIG. 1C), the fixation device 12 is inserted into the heart 200 and navigated toward the target valve to attempt to grasp the leaflets LF. During this initial approach, the distal elements 20 and the proximal fixation elements 40 are actuated to an open configuration, thereby defining a graspable volume of space 404 (FIG. 3A) therebetween. Here, the goal is to position the fixation device 12 such that the leaflets LF are sufficiently inserted into the graspable volume of space 404. However, achieving and confirming this optimal positioning is highly challenging. The leaflets LF are in constant, rapid motion due to the dynamic cardiac cycle of the beating heart 200, and the presence of flowing blood further obscures direct visualization.

To complete the repair, the procedure advances to a second stage where, upon confirming that the leaflets LF are adequately positioned within the graspable volume of space 404, the proximal fixation elements 40 are actuated to a closed (or lowered) position. The actuation compresses and secures the leaflets LF between the distal elements 20 and the proximal fixation elements 40. Finally, in a third stage, the distal elements 20 are actuated to a closed position, drawing the grasped leaflets LF together to coapt the valve and establish a mechanical bridge that reduces regurgitation. Once the clinician confirms a satisfactory reduction in regurgitant flow, the shaft 900 is released from the fixation device 12, leaving the fixation device 12 implanted at the target site.

Referring now to FIGS. 3A and 3B, enlarged views of a fixation device 500 in an open or insertion configuration are provided to further illustrate the leaflet capturing mechanics. As depicted, the fixation device 500 includes a pair of distal elements 702 and a corresponding pair of proximal fixation elements 610. The pair of distal elements 702 include a first distal element 702, 702a and a second distal element 702, 702b. The pair of proximal fixation elements 610 includes a first proximal fixation element 610, 610a and a second proximal fixation element 610, 610b. In this insertion stage, the distal elements 702 are deployed radially outward to prepare for tissue engagement, while the proximal fixation elements 610 are maintained in a raised, unactuated position running generally parallel to the central longitudinal axis of the device and the shaft 900.

The specific open arrangement establishes the graspable volume of space 404, shown outlined in dotted lines on the right side of the fixation device 500. The graspable volume of space 404 is defined as the three-dimensional capture zone bounded generally by an engagement surface of the raised proximal fixation element 610, an opposing engagement surface of the extended distal element 702, and a central body portion of the fixation device 500. For a secure, durable repair, a native leaflet LF should be navigated deeply and securely into the graspable volume of space 404 such that a free edge of the leaflet LF is positioned sufficiently deep (e.g., more than 40%, 60%, 80%, or 100% insertion) within the fixation device 500 and far enough into the graspable volume of space 404 before the proximal fixation element 610 is deployed downwardly to clamp the leaflet LF. If the leaflet LF occupies only a superficial or marginal portion (e.g., less than 40%, 60%, 80%, or 100% insertion) of the graspable volume of space 404, or if the free edge FE has not reached a target depth within the clip prior to closing, the resulting grip may be tenuous, increasing the risk of the leaflet LF slipping out under the high-pressure hemodynamic forces of the functioning heart. The target depth may be 20%, 40%, 60%, 80%, 100%, or any value therebetween. Consequently, accurately confirming the extent to which the leaflet LF fills the graspable volume of space 404 prior to actuation is an important step in ensuring a successful TEER.

To overcome such challenge and confirm adequate insertion, as shown specifically in FIG. 3B, the interventional system 1100 incorporates the sensor assembly 400. The sensor assembly 400 is disposed proximal to the proximal fixation elements 610, such as on a distal end portion of a delivery catheter 850 coupled to the shaft 900.

From the elevated vantage point, the sensor assembly 400 maintains a clear line of sight to direct emitted signals distally into the graspable volume of space 404 and toward the distal elements 702.

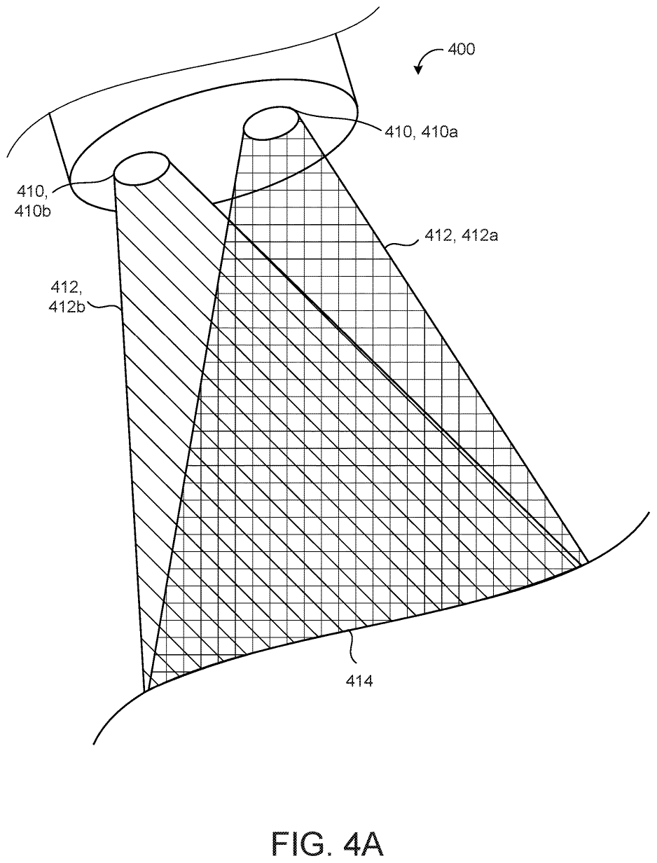

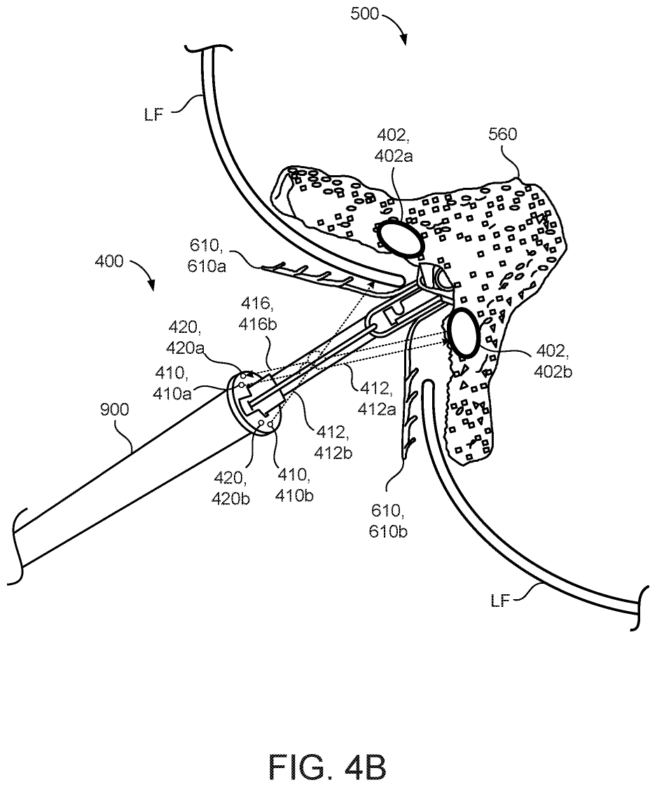

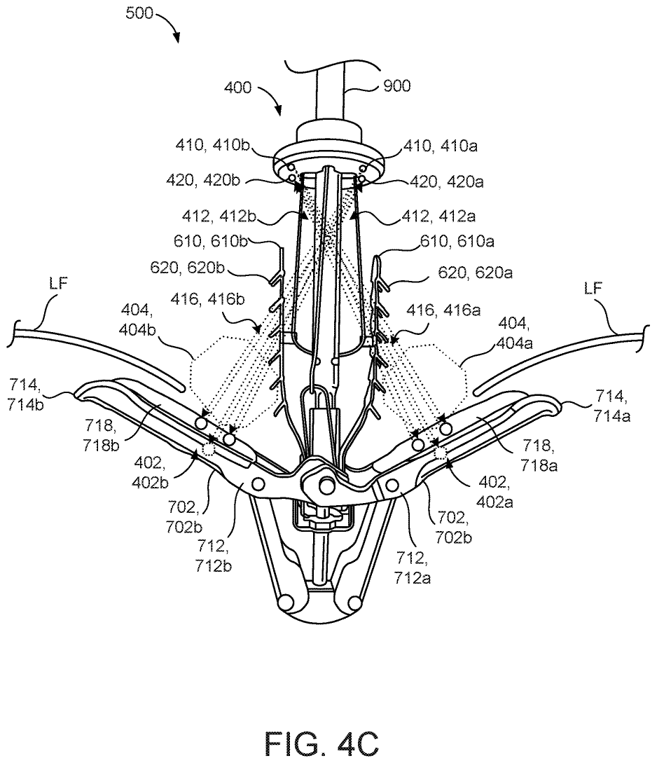

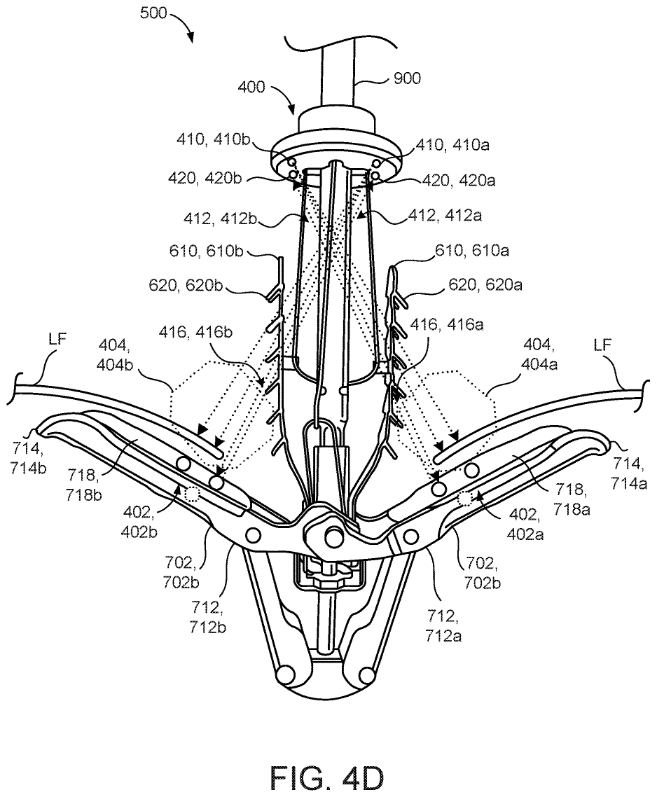

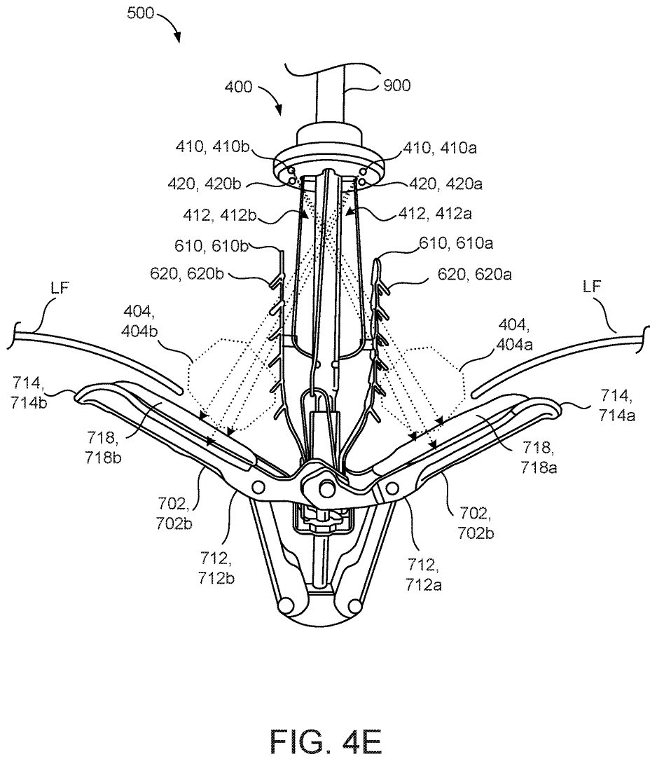

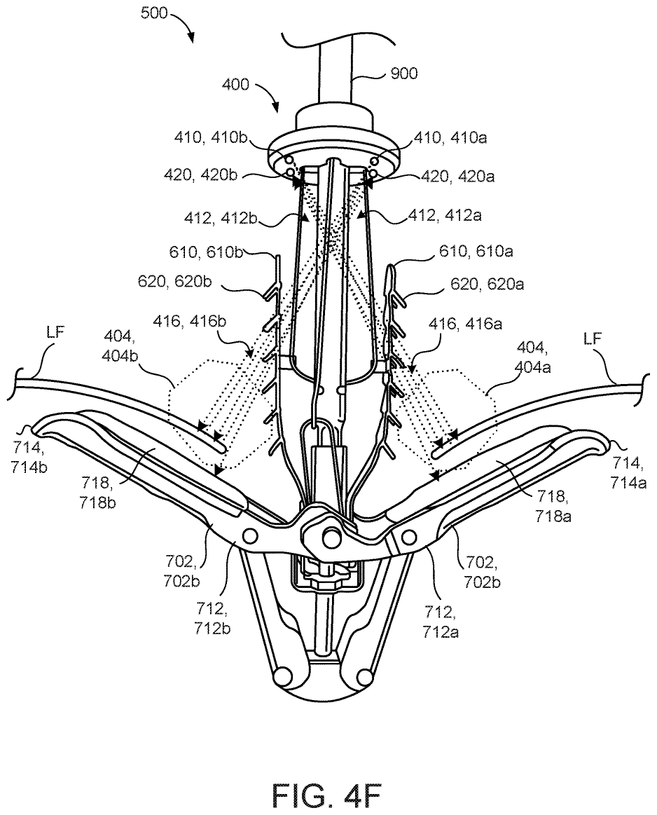

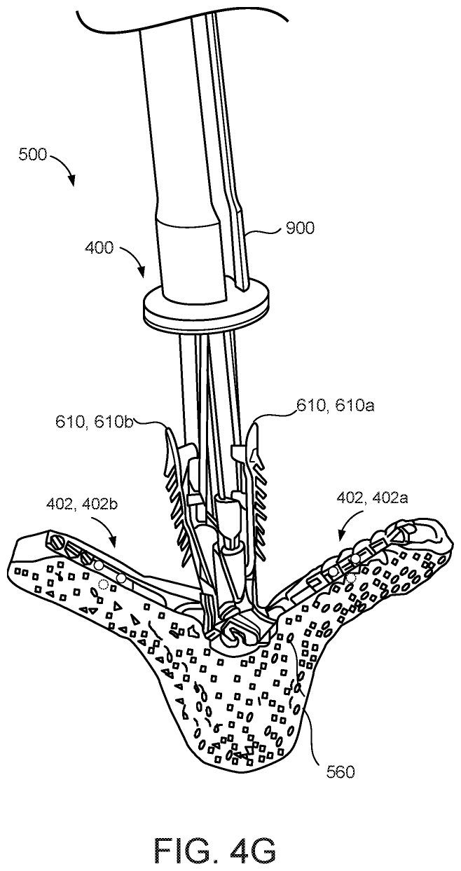

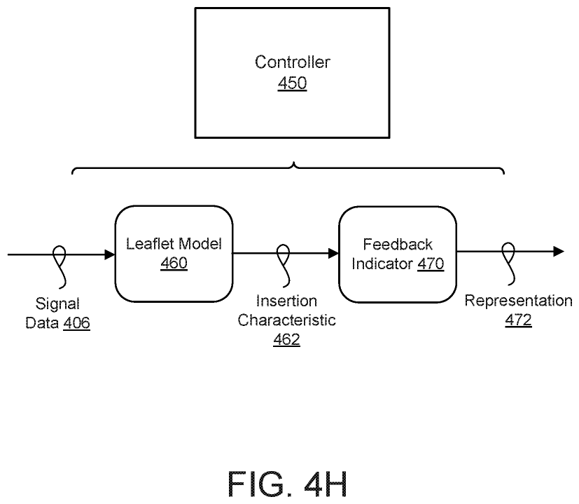

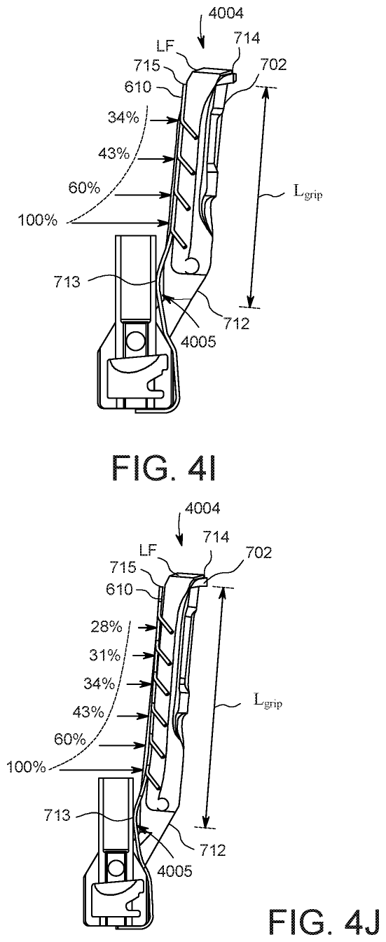

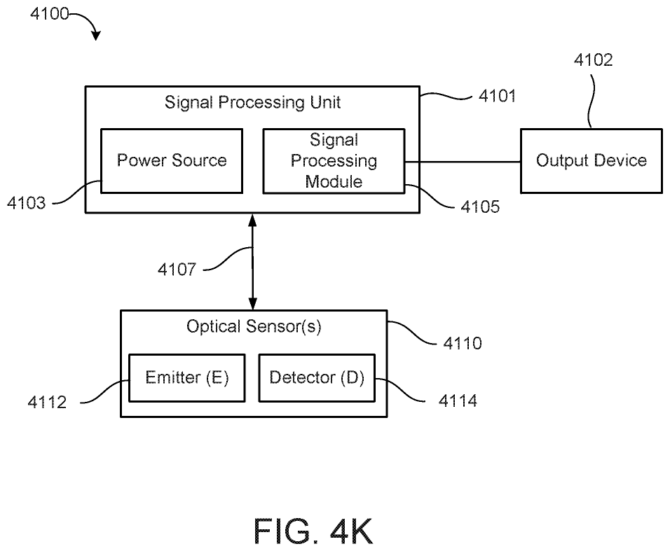

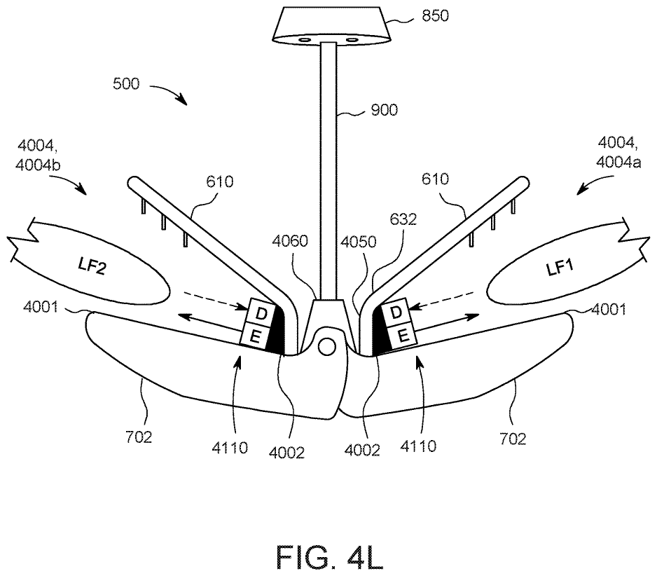

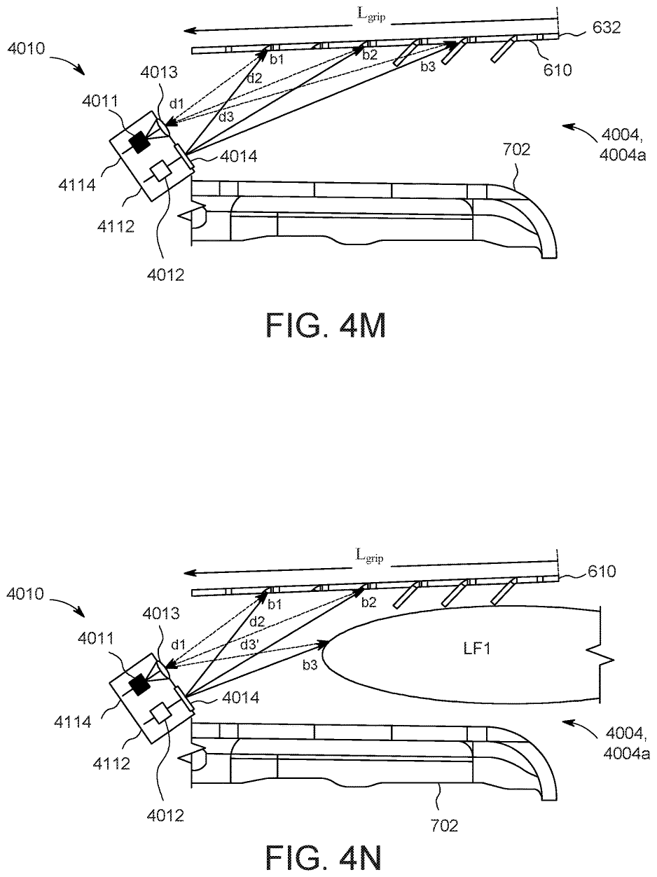

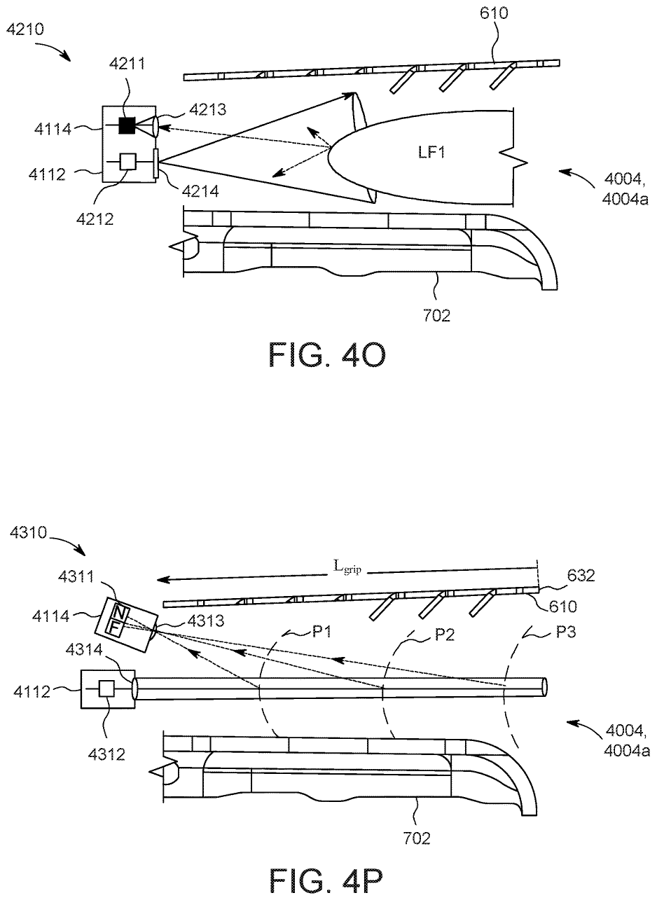

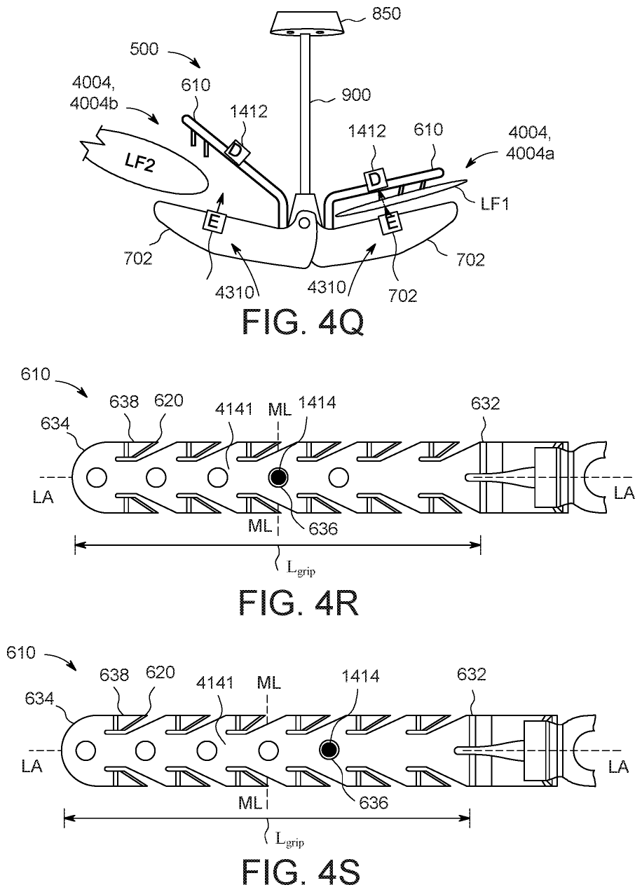

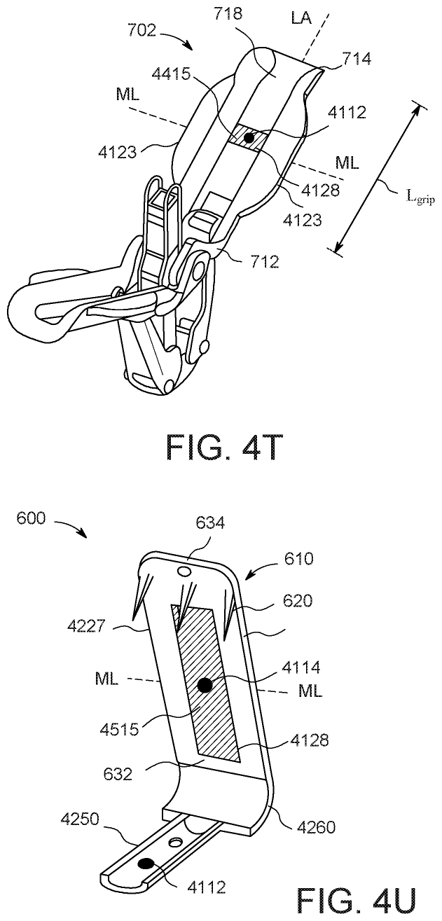

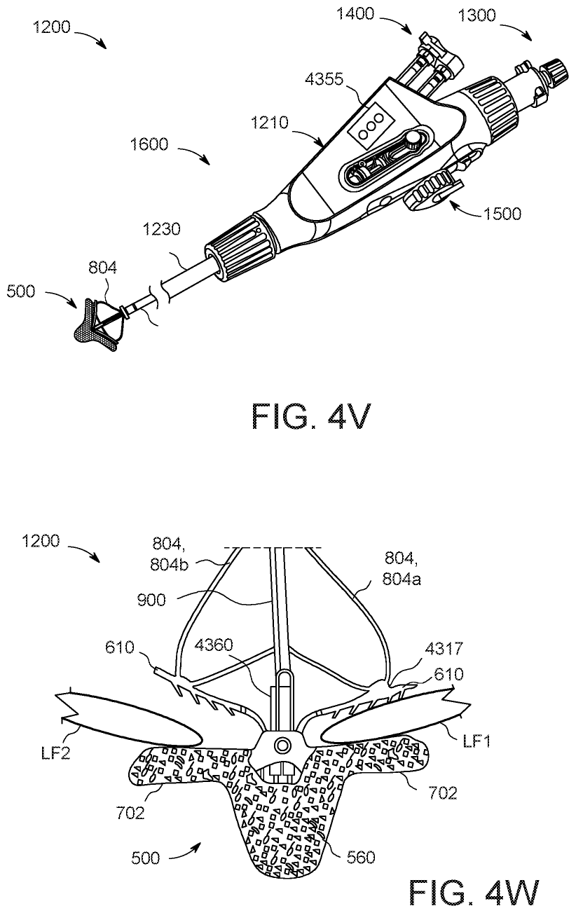

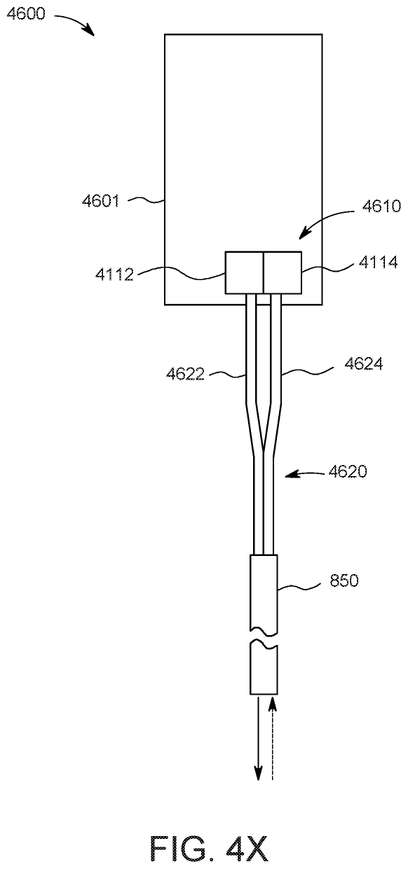

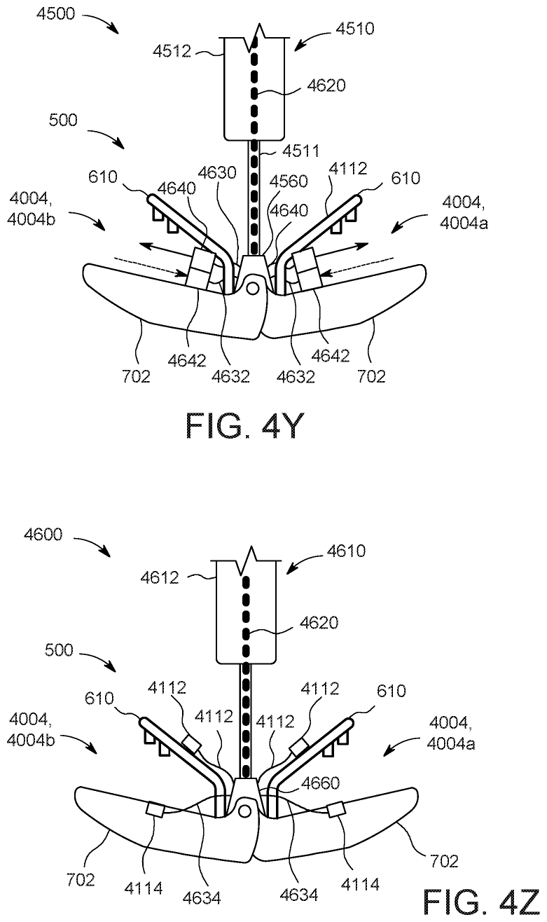

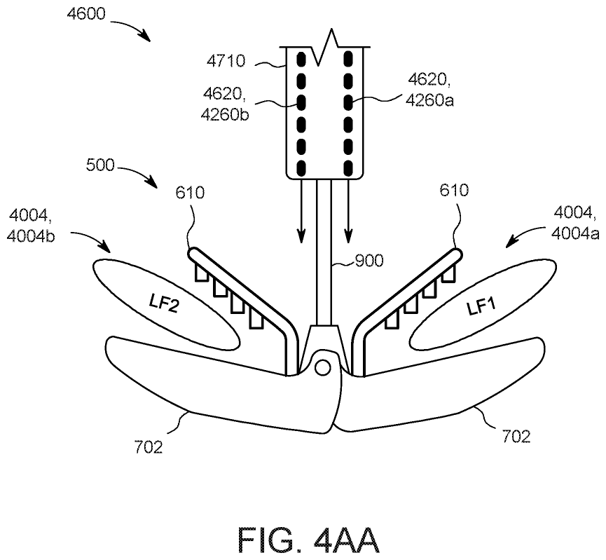

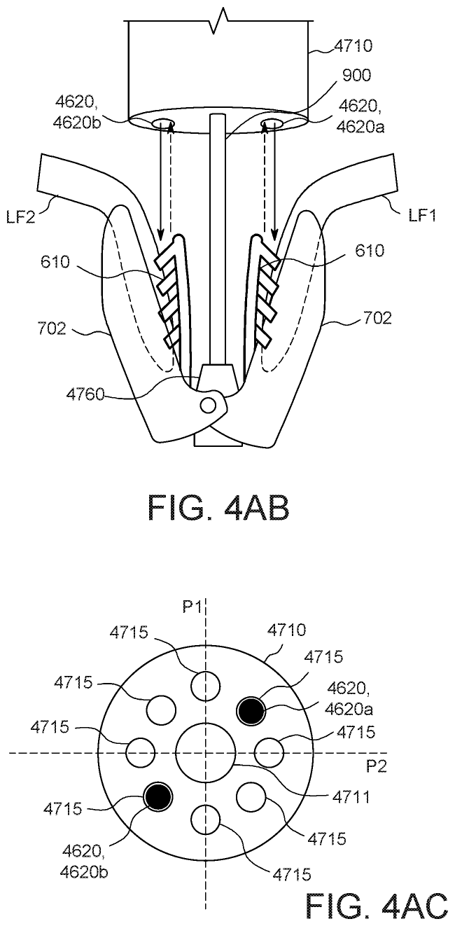

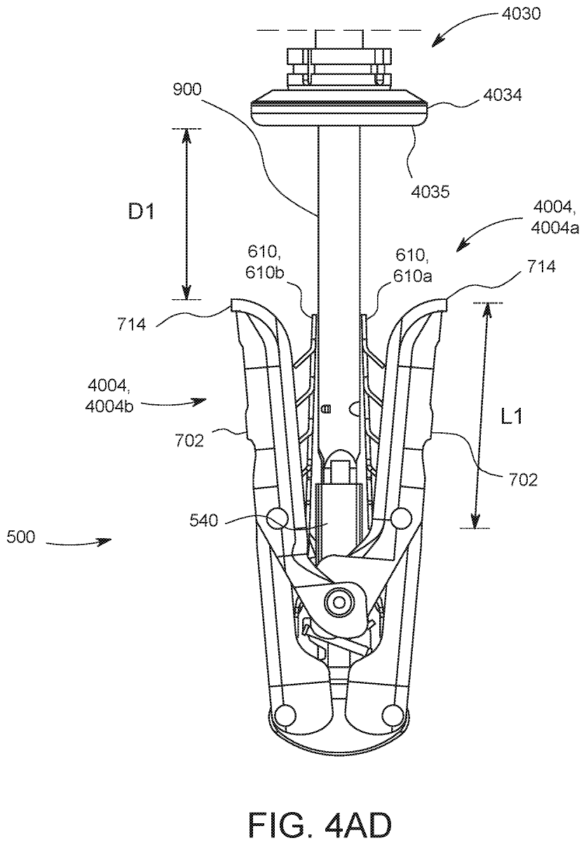

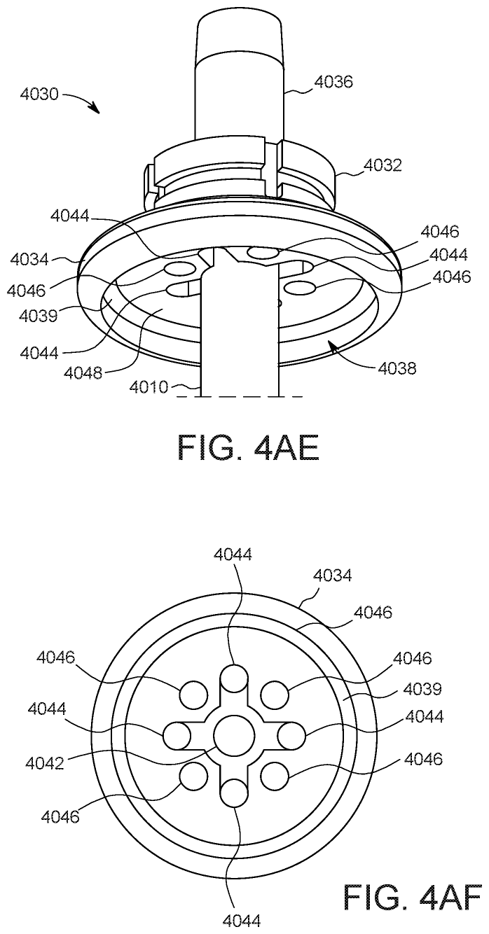

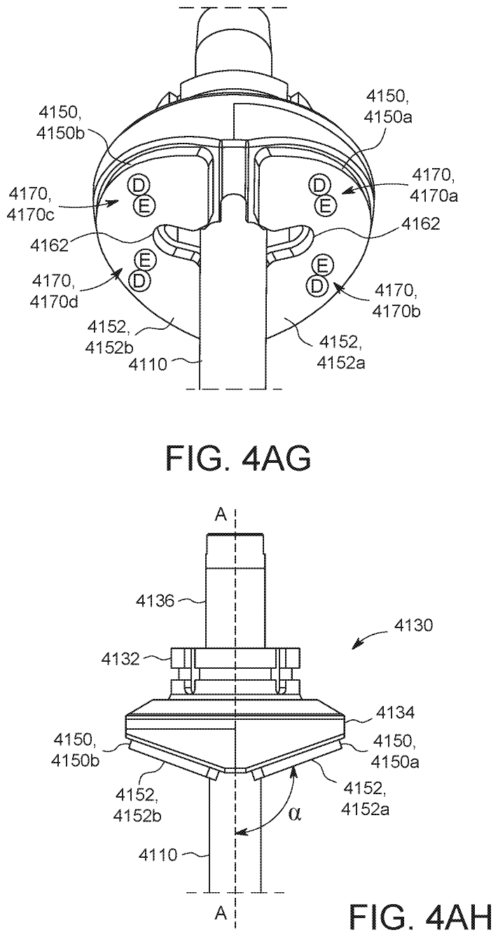

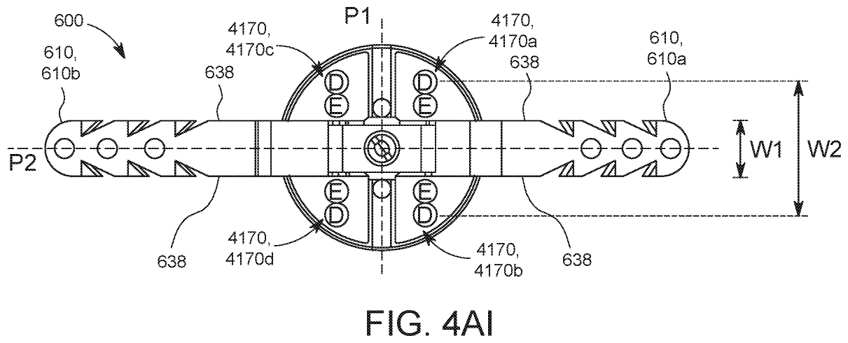

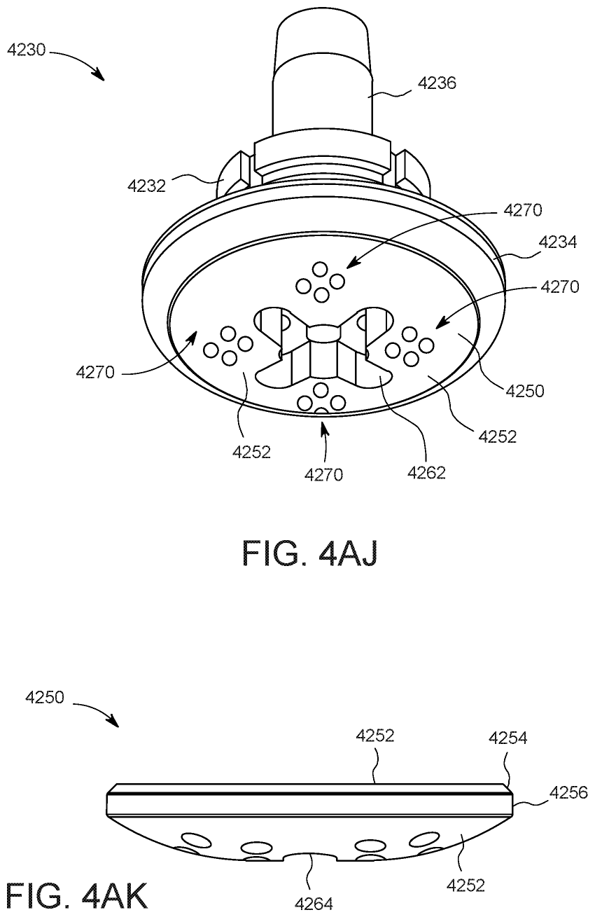

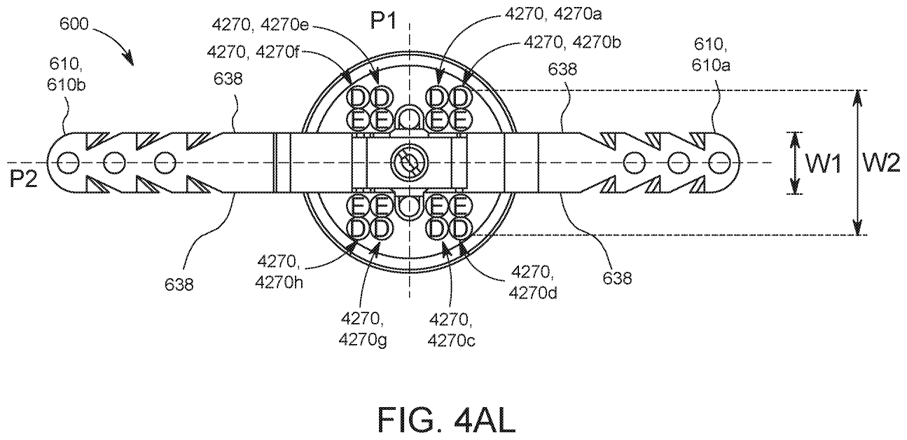

Referring now to FIG. 4A, in some implementations, the sensor assembly 400 includes a first signal emitter 410, 410a and a second signal emitter 410, 410b. The first signal emitter 410a is configured to project a first emitted signal 412, 412a along a first emission path. Similarly, the second signal emitter 410b is configured to project a second emitted signal 412, 412b along a second emission path. The first signal emitter 410a and the second signal emitter 410b may also function as corresponding first and second signal receivers, or they may be paired with discrete receiving components to detect the reflected signals returning from the leaflet tissue or the fixation device. The first emitted signal 412a and the second emitted signal 412b may project distally and widen to form a combined sensing area 414. In this configuration, the first emitted signal 412a and the second emitted signal 412b partially overlap within the combined sensing area 414 to provide comprehensive coverage of the graspable volume of space 404. The dual-signal or multi-signal arrangement allows the interventional system 1100 to more accurately assess the geometry and positioning of the leaflet tissue.

Referring now to FIGS. 4B-4G, the interventional system 1100 includes the sensor assembly 400 configured to detect leaflet insertion during the TEER procedure.

The sensor assembly 400 provides real-time, objective feedback to a clinician regarding a position of the leaflet LF of a heart valve relative to the fixation device 500. This feedback improves grasping outcomes and significantly reduces the need for repositioning during the procedure. The sensor assembly 400 is disposed on a distal end portion of the shaft 900 and is configured to emit the emitted signals 412 toward the fixation device 500 and receive reflected signals 416 back from the fixation device 500 or the leaflet LF.

As illustrated across FIGS. 4B-4G, the fixation device 500 includes a first distal fixation element 702, 702a and a second distal fixation element 702, 702b. The first distal fixation element 702a includes a first engagement surface 718, 718a, and the second distal fixation element 702b includes a second engagement surface 718, 718b.

Within the open position, the first engagement surface 718a and the second engagement surface 718b orient generally toward the sensor assembly 400. For example, when the first distal fixation element 702a and the second distal fixation element 702b reach an angle of approximately 120 degrees relative to one another, the first engagement surface 718a and the second engagement surface 718b align with an emission path of the sensor assembly 400.

A distal end portion of the shaft 900 (or the delivery catheter 850) supports the sensor assembly 400. Placement of the sensor assembly 400 at the distal end portion allows the sensor assembly 400 to maintain a clear line of sight toward the graspable volume of space 404. Proper alignment between the sensor assembly 400 and the first engagement surface 718a and the second engagement surface 718b facilitates accurate signal reception. The sensor assembly 400 may monitor the graspable volume of space 404 to identify a specific location of the free edge FE of the leaflet LF during a grasping sequence. Such an arrangement allows the sensor assembly 400 to detect the reflected signals 416 returning from the first engagement surface 718a and the second engagement surface 718b without interference from other components. In some examples, the sensor assembly 400 is positioned such that the emitted signals 412 travel distally through the graspable volume of space 404 to strike the first engagement surface 718a and the second engagement surface 718b. Moving the first distal fixation element 702a and the second distal fixation element 702b to different angles may vary the intensity of the signals received by the sensor assembly 400.

The fixation device 500 further includes a first proximal fixation element 610, 610a and a second proximal fixation element 610, 610b. As illustrated in the arrangement of FIGS. 4A-4H, the first distal fixation element 702a and the second distal fixation element 702b reside distal to the sensor assembly 400. Conversely, the first proximal fixation element 610a and the second proximal fixation element 610b reside proximal to the sensor assembly 400. In the open position, the first proximal fixation element 610a and the first distal fixation element 702a define a first graspable volume of space 404, 404a between a first engagement surface 620, 620a of the first proximal fixation element 610a and the first engagement surface 718, 718a of the first distal fixation element 702a. For example, when the first distal fixation element 702a extends radially outward at an angle of approximately sixty degrees from the shaft 900, the first graspable volume of space 404a forms a wedge-shaped capture zone configured to receive a portion of the leaflet LF.

Similarly, the second proximal fixation element 610b and the second distal fixation element 702b define a second graspable volume of space 404, 404b between a second engagement surface 620, 620b of the second proximal fixation element 610b and the second engagement surface 718, 718b of the second distal fixation element 702b. The spatial relationship between the sensor assembly 400 and the first graspable volume of space 404a and the second graspable volume of space 404b enables the first signal emitter 410a and the second signal emitter 410b to direct the emitted signals 412 into these zones to detect the presence of heart valve tissue. The emission paths of the signals 412 may be configured in either a non-crossing or a crossing arrangement. In a non-crossing configuration, the first signal emitter 410a directs a signal generally straight toward the corresponding first graspable volume of space 404a, and the second signal emitter 410b directs a signal generally straight toward the corresponding second graspable volume of space 404b. Alternatively, in a crossing or diagonal configuration, the first signal emitter 410a directs a signal diagonally toward the opposite second graspable volume of space 404b, while the second signal emitter 410b directs a signal diagonally toward the opposite first graspable volume of space 404a. Utilizing a crossing configuration may advantageously optimize the angle of incidence against the leaflet tissue and better accommodate compact packaging constraints within the distal end portion of the delivery catheter. Conversely, a non-crossing configuration can beneficially simplify optical alignment and the subsequent signal processing. Placement of the sensor assembly 400 proximal to the first graspable volume of space 404a and the second graspable volume of space 404b allows a signal receiver 420 to monitor leaflet insertion depth from an elevated vantage point before a clinician actuates the first proximal fixation element 610a and the second proximal fixation element 610b toward a closed position.

To facilitate insertion detection, the fixation device 500 includes one or more reflective markers 402 disposed on the first engagement surface 718a and the second engagement surface 718b. The one or more reflective markers 402 reflect the emitted signals 412 emitted by the sensor assembly 400, generating the reflected signals 416 that enable detection of a position of the leaflet LF. As shown in FIG. 4B, in some examples, a single reflective marker 402a, 402b, such as an oval or pad, may be disposed on the first distal fixation element 702a and the second distal fixation element 702b. Alternatively, as shown in the perspective view of FIG. 4C, the one or more reflective markers 402 may include a linear array of discrete reflective markers, such as dots or small circles. The first engagement surface 718a includes a first proximal region 712, 712a and a first distal region 714, 714a. The second engagement surface 718b includes a second proximal region 712, 712b and a second distal region 714, 714b. The first proximal region 712a and the first distal region 714a represent specific zones along the first engagement surface 718a of the first distal fixation element 702a. The first proximal region 712a is located closer to the center axis of the fixation device 500 and the pivot point of the first distal fixation element 702a, serving as the inner-most capture area for the heart valve leaflet. The first distal region 714a is located toward the free end of the first distal fixation element 702a, representing the outer-most capture area. The reflective markers 402 may be disposed exclusively in the first proximal region 712a, exclusively in the first distal region 714a, or across both regions. Placing markers exclusively in the first proximal region 712a is advantageous as it minimizes marker material while providing a clear, binary confirmation of a deep, secure grasp. However, providing an arrangement of the one or more reflective markers 402 (such as a single line, multiple parallel lines, or a scattered array) that spans from the first proximal region 712a to the first distal region 714a is highly advantageous. This span enables the sensor assembly 400 to differentiate between a leaflet LF that is only marginally inserted at the first distal region 714a and a leaflet that is deeply seated at the first proximal region 712a. It is not strictly necessary for the reflective markers 402 to cover the entirety of the engagement surface between these regions; the system may successfully function with the markers 402 covering a minimum of 5%, 10%, 25%, or 50% of the surface area or longitudinal distance between the proximal and distal regions, beneficially allowing for granular insertion feedback while conserving surface area for tissue ingrowth.

In the linear array implementation of FIG. 4C, the one or more reflective markers 402 form a pattern extending from the first proximal region 712a and the second proximal region 712b toward the first distal region 714a and the second distal region 714b. The linear array includes discrete markers spaced at specific intervals, such as every one millimeter, to facilitate highly granular and precise measurement of a depth of the leaflet LF. As the leaflet LF enters the first graspable volume of space 404a or the second graspable volume of space 404b, the leaflet LF sequentially covers the discrete markers. For example, a leaflet LF that covers three markers in a five-marker array indicates a deeper insertion than a leaflet LF covering only the inner-most marker. By monitoring which discrete markers in the linear array the leaflet LF obscures, the sensor assembly 400 provides exact insertion data to a controller 450 for determining an insertion characteristic. The granular feedback allows a clinician to quantify leaflet LF penetration before moving the first proximal fixation element 610a or the second proximal fixation element 610b to the closed position.

The one or more reflective markers 402 reside on the first engagement surface 718a and the second engagement surface 718b in an arrangement that prevents the first proximal fixation element 610a and the second proximal fixation element 610b from obstructing the emission path between the sensor assembly 400 and the one or more reflective markers 402. For example, the one or more reflective markers 402 may reside on lateral edges of the first engagement surface 718a and the second engagement surface 718b, while the first proximal fixation element 610a and the second proximal fixation element 610b occupy a central longitudinal area of the fixation device 500. This spatial separation maintains a clear line of sight for the emitted signals 412 and the reflected signals 416. Consequently, the sensor assembly 400 receives the reflected signals 416 without interference from the first proximal fixation element 610a and the second proximal fixation element 610b whether the first proximal fixation element 610a and the second proximal fixation element 610b occupy an open position or a closed position.

Such a configuration allows the sensor assembly 400 to monitor the graspable volume of space 404 continuously during the grasping sequence. For instance, the first signal emitter 410a directs the first emitted signal 412a past the first proximal fixation element 610a to strike the one or more reflective markers 402 located at the periphery of the first distal fixation element 702a. This clear path of travel for the first emitted signal 412a and the reflected signal 416 enables the signal receiver 420 to detect precise changes in signal intensity as a heart valve leaflet LF enters the graspable volume of space 404.

The sensor assembly 400 may include the first signal emitter 410a, the second signal emitter 410b, a first signal receiver 420, 420a, and a second signal receiver 420, 420b. The first signal emitter 410a directs the first emitted signal 412a along a first emission path toward the single reflective marker 402a disposed on the first engagement surface 718a. Simultaneously, the second signal emitter 410b directs the second emitted signal 412b along a second emission path toward the single reflective marker 402b disposed on the second engagement surface 718b. In some implementations, the first signal emitter 410a is configured to direct the first emitted signal 412a toward the one or more reflective markers 402 when the first distal fixation element 702a is in the open position. The first signal receiver 420a detects a first reflected signal 416, 416a returning from the first engagement surface 718a, while the second signal receiver 420b detects a second reflected signal 416, 416b returning from the second engagement surface 718b.

For example, the sensor assembly 400 may detect a high-intensity reflection from the first engagement surface 718a indicating no leaflet presence, while detecting a low-intensity reflection from the second engagement surface 718b indicating deep leaflet insertion.

Such independent monitoring of the first distal fixation element 702a and the second distal fixation element 702b facilitates simultaneous, side-by-side verification of insertion for multiple leaflets LF. The dual-channel feedback provides the clinician with precise data to confirm a balanced and secure grasp across the line of coaptation LOC before final device deployment.

The interventional system 1100 may leverage a principle of progressive occlusion to determine a depth of leaflet insertion. As the leaflet LF moves into the graspable volume of space 404, the leaflet LF progressively occludes the one or more reflective markers 402 from a perspective of the sensor assembly 400. Because heart valve tissue reflects an emitted signal differently than the one or more reflective markers 402, a reflected signal intensity changes proportionally to an amount of marker surface area the leaflet LF covers. For example, the one or more reflective markers 402 may include a material with higher infrared reflectivity than native tissue, such that the signal receiver 420 detects a decrease in signal return as the leaflet LF covers the one or more reflective markers 402.

A progressive occlusion sequence is illustrated in the progression from FIG. 4C to FIG. 4D. In FIG. 4C, the leaflets LF reside entirely outside the graspable volume of space 404. Consequently, the emitted signals 412 strike an entirety of the exposed reflective markers 402, resulting in a maximum intensity of the reflected signals 416. In some implementations, the one or more reflective markers 402 are arranged such that the reflected signals 416 are proportional to an amount of occlusion of the one or more reflective markers 402 by the leaflet LF of the heart valve positioned in the graspable volume of space 404. In FIG. 4D, the leaflets LF demonstrate marginal insertion, just crossing a threshold into the graspable volume of space 404 and blocking only a proximal region of the one or more reflective markers 402. This initial coverage results in a slight, detectable drop in reflected signal intensity. In FIG. 4D, the leaflets LF reach a position of insertion, occluding some of the one or more reflective markers 402. A significant drop in the reflected signals 416 indicates to a clinician that the leaflets LF reached a suitable depth for a secure, durable grasp.

In some implementations, the interventional system 1100 may detect leaflet insertion without relying on the one or more reflective markers 402. In such implementations, the sensor assembly 400 may be configured to sense reflections directly off of the leaflet LF tissue itself as the leaflet LF enters the graspable volume of space 404. The first signal emitter 410a may be arranged to direct the first emitted signal 412a along an emission path toward the graspable volume of space 404 defined by the proximal fixation elements 610 and the distal elements 702 when in the open position.

The first signal receiver 420a may be arranged to receive the first reflected signal 416a of the first emitted signal 412a off of the leaflet LF of the heart valve when positioned within the graspable volume of space 404. In some examples, the first signal emitter 410a may emit a diverging beam covering the graspable volume of space 404, or a focused beam that scans across the capture zone.

The optical properties of heart valve leaflet tissue may enable reliable detection using the direct tissue sensing approach. Leaflet tissue may reflect infrared light at a higher intensity than blood, which tends to absorb infrared wavelengths. This differential reflectance may allow the sensor assembly 400 to distinguish between the presence and absence of leaflet tissue within the graspable volume of space 404. Wavelengths in the range of about 700 nm to about 900 nm or about 1000 nm to about 1700 nm may provide suitable tissue-to-blood contrast for leaflet detection.

In the direct tissue sensing approach, the intensity of the reflected signals 416 may change proportionally to the position of the leaflet LF within the graspable volume of space 404. As the leaflet LF moves deeper into the graspable volume of space 404, a larger surface area of leaflet tissue becomes available to reflect the emitted signals 412 back toward the signal receiver 420, resulting in increased reflected signal intensity. The controller 450 may process signal data 406 to distinguish tissue reflections from background reflections, such as by comparing received signal intensity against a baseline threshold established when no leaflet tissue is present. In some implementations, the controller 450 may employ temporal filtering, spectral analysis, or machine learning algorithms to improve detection accuracy. The direct tissue sensing approach may offer certain advantages in some implementations, such as providing leaflet position information without requiring the one or more reflective markers 402 on the fixation device 500, enabling continuous position feedback rather than discrete position indications, and reducing sensitivity to leaflet orientation within the graspable volume of space 404. In some implementations, the interventional system 1100 may combine the direct tissue sensing approach with the reflective marker approach, where the controller 450 processes the signal data 406 from both sources to determine an insertion characteristic 462.

To optimize this detection in a dynamic cardiovascular environment, the emitted signals 412a, 412b may include infrared light, and the one or more reflective markers 402 may be configured to highly reflect infrared light. Infrared light penetrates blood and surrounding tissue to a greater extent than visible light, enabling highly reliable detection of the leaflet LF position without interference from ambient light sources or the constant flow of blood in the field. The signal emitters 410a, 410b may be configured to emit near-infrared light having a wavelength of about 700 nm to about 900 nm, or about 1000 nm to about 1700 nm, depending on the optical properties of the selected markers 402 and the expected distance between the sensor assembly 400 and the distal fixation elements 702.

The reflective markers 402 may include a biocompatible material selected from the group consisting of gold, titanium nitride, and alumina with a metallic coating.

Gold provides exceptional reflectivity in the infrared spectrum and is highly biocompatible for long-term implantation. As shown in FIGS. 4B and 4G, the fixation device 500 may further include a covering 560 disposed over the distal fixation elements 702 to minimize tissue trauma and promote cellular ingrowth. In such implementations, the one or more reflective markers 402 are configured to at least partially protrude through the covering 560. This arrangement ensures the reflective surfaces remain exposed to the emitted signals 412 and can successfully bounce the reflected signals 416 back to the signal receivers 420, unimpeded by the fabric or mesh of the covering 560.

Alternatively, the covering 560 may include one or more through-holes aligned with the one or more reflective markers 402, such that the markers 402 sit flush with or recessed beneath the covering 560 but remain optically exposed.

Referring now to FIG. 4H, in some implementations, the data processing hardware 2112 and the memory hardware 2114 of the computing device 2100 executes the controller 450. The controller 450 employs a leaflet model 460 configured to receive signal data 406 from the sensor assembly 400 corresponding to the reflected signal 412. The signal data 406 may include analog or digital information representing the intensity, wavelength, or time-of-flight of the reflected signal 412. The leaflet model 460 processes the signal data 406 received from the sensor assembly 400 and determines an insertion characteristic 462 based on processing the signal data 406. The insertion characteristic 462 provides an indication of a position of the leaflet LF within the graspable volume of space 2002. For example, the insertion characteristic 462 identifies a specific location of the free edge FE of the leaflet LF relative to a base of the distal elements 520. The insertion characteristic 462 provides quantitative data regarding a depth of leaflet LF penetration. In some implementations, the insertion characteristic 462 provides a numeric value representing a distance in millimeters from a fixed reference point, such as a coupling member or a proximal region of the engagement surface 718. In other implementations, the insertion characteristic 462 represents a percentage of the engagement surface 718 currently covered or obscured by the leaflet LF. For example, a value of seventy percent would indicate the leaflet LF covers a majority of the available surface area of the engagement surface 718.

In implementations where the fixation device 500 incorporates the one or more reflective markers 402, the leaflet model 460 evaluates changes in signal intensity. Here, the leaflet model 460 may determine the insertion characteristic 462 by interpreting a decrease in the reflected signal 412 as an increase in insertion of the leaflet LF relative to the engagement surface 718. As the leaflet LF moves deeper into the graspable volume of space 404, the leaflet LF progressively occludes the one or more reflective markers 402. Consequently, the signal receiver 420 detects a reduced amount of reflected light.

For instance, if the leaflet LF blocks four out of five reflective markers 402, the leaflet model 460 recognizes a corresponding eighty percent reduction in reflected signal intensity and correlates the reduction with a specific insertion depth.

In other implementations, where the sensor assembly 400 detects reflections directly off of heart valve tissue, the leaflet model 460 applies an inverse logic. Here, the leaflet model 460 determines the insertion characteristic 462 by interpreting an increase in the reflected signal 412 as an increase in insertion of the leaflet LF relative to the distal elements 702. Leaflet tissue may reflect signals differently than blood. Thus, as the leaflet LF enters the graspable volume of space 402, a larger surface area of tissue becomes available to reflect the emitted signal 412 back toward the signal receiver 420. The leaflet model 460 analyzes the signal data 406 to distinguish between reflections from tissue and reflections from background structures or fluid. An increase in an amplitude of the reflected signal 412 signals that a greater portion of the leaflet LF resides within the graspable volume of space 402. For example, a high-intensity return signal indicates that the leaflet LF is fully seated against the engagement surface 718. Such logic allows a clinician to verify a grasp before actuating the proximal elements 610.

The feedback indicator 470 presents a representation 472 of the insertion characteristic 462 to provide real-time guidance during a procedure. In some implementations, the feedback indicator 470 includes one or more lights integrated with the interventional system 1100, such as within the delivery catheter handle 1210. In other implementations, the feedback indicator 470 is separate from and in communication with the interventional system 1100, such as a monitor or screen displaying a graphical user interface.

The representation 472 may include a visual representation, such as a graphical icon, a numeric value, a bar graph, or a color-coded light. For example, the feedback indicator 470 may display a first color (e.g., green) indicating a threshold insertion of the leaflet LF, a second color (e.g., yellow) indicating marginal insertion, or a third color (e.g., red) indicating no insertion or insufficient insertion. In some examples, the feedback indicator 470 displays a numeric value from 0 to 100 representing the percentage of the engagement surface 718 covered by the leaflet LF. The representation 472 may also include a haptic representation, such as a vibration pattern delivered through the delivery catheter handle 1210, or an audible representation, such as a tone that changes in pitch or frequency as the leaflet LF moves deeper into the graspable volume of space 404. Such indications allow a clinician to verify that the leaflet LF is properly positioned relative to the distal elements 702 before actuating the proximal elements 610 toward the closed position. The real-time data helps confirm that the leaflet LF is fully (or sufficiently) seated within the graspable volume of space 402, which reduces the likelihood of a partial grasp or leaflet slip-out during the repair.

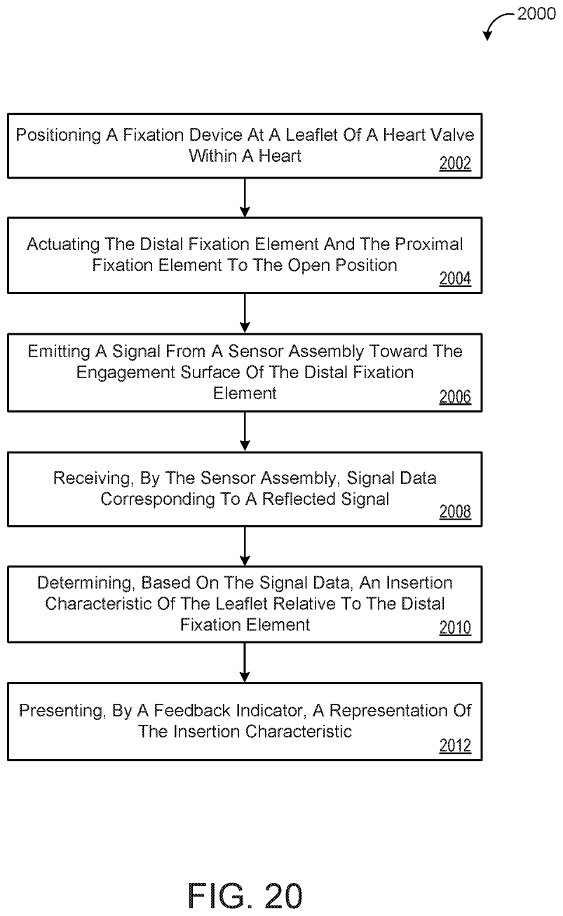

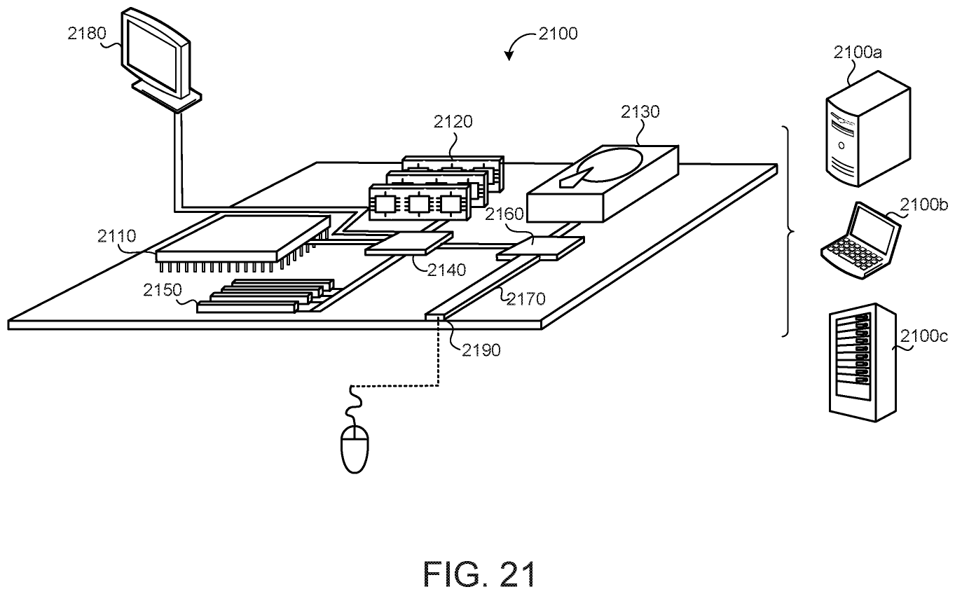

FIG. 20 illustrates an example arrangement of operations for a method 2000 of detecting insertion of the leaflet LF during a transcatheter edge-to-edge repair procedure. At operation 2002, the method 2000 includes positioning the fixation device 500 at the leaflet LF of a heart valve within the heart 200. The fixation device 500 includes the distal elements 702 having the engagement surface 718. The distal elements 702 are actuatable between an open position and a closed position. The fixation device 500 also includes the proximal elements 610 having the engagement surface 620. The proximal elements 610 are actuatable between an open position and a closed position.

At operation 2004, the method 2000 includes actuating the distal elements 610 and the proximal elements 702 to the open position. While in the open position, the fixation device 500 defines the graspable volume of space 402 between the engagement surface 620 of the proximal elements 610 and the engagement surface 718 of the distal elements 702. At operation 2006, the method 2000 includes emitting the emitted signal 412 from the signal emitter 410 of the sensor assembly 400 toward the engagement surface 718 of the distal elements 702. In some implementations, the signal emitter 410 is configured to direct the emitted signal 412 along an emission path toward the one or more reflective markers 402 disposed on the engagement surface 718 of the distal elements 702. For example, the signal emitter 410 emits near-infrared light having a wavelength of about 700 nm to about 900 nm or about 1000 nm to about 1700 nm. At operation 2008, the method 2000 includes receiving, by the signal receiver 420 of the sensor assembly 400, the signal data 406 corresponds to the reflected signal 416. The signal receiver 420 is configured to receive the reflected signal 416 of the emitted signal 412 off of the one or more reflective markers 402 or off of the leaflet LF.

At operation 2010, the method 2000 includes determining, via the controller 450 based on the signal data 406, the insertion characteristic 462 of the leaflet LF relative to the distal elements 702. In implementations where the fixation device 500 includes the one or more reflective markers 402, the controller 450 determines the insertion characteristic 462 by interpreting a decrease in the reflected signal 416 as an increase in leaflet insertion relative to the engagement surface 718 of the distal elements 702. Here, the decrease occurs because the leaflet LF progressively occludes the one or more reflective markers 402 as the leaflet LF moves deeper into the graspable volume of space 404. In other implementations, the signal emitter 410 is arranged to direct the emitted signal 412 toward the graspable volume of space 404 and the signal receiver 420 is arranged to receive the reflected signal 412 off of the leaflet LF. Here, the controller 450 determines the insertion characteristic 462 by interpreting an increase in the reflected signal 412 as an increase in leaflet insertion relative to the distal elements 610.

At operation 2012, the method 2000 includes presenting, by the feedback indicator 470, the representation 472 of the insertion characteristic 462. The representation 472 provides real-time confirmation of the position of the leaflet LF.

Presenting the representation 472 of the insertion characteristic 462 includes displaying a numeric value, such as a percentage of surface coverage or a distance measurement. The feedback indicator 470 also displays at least one of a first color indicating a threshold insertion of the leaflet LF, a second color indicating marginal insertion of the leaflet LF, or a third color indicating no insertion of the leaflet LF. The representation 472 of the insertion characteristic 462 may also include a haptic representation, such as a vibration delivered to the delivery catheter handle 1210.

In some examples, the method 2000 includes identifying a baseline for the sensor assembly 400. Identifying the baseline may include actuating the distal elements 702 to the closed position to confirm an absence of the reflected signal 416 and then actuating the distal elements 702 to the open position to confirm a presence of the reflected signal 416. If the representation 472 indicates insufficient leaflet insertion, the method 2000 includes adjusting at least one of an orientation or a position of the distal elements 702 until the insertion characteristic 462 satisfies a threshold. The adjustment may involve actuating the proximal elements 610 toward the open position and repositioning the fixation device 500 relative to the leaflet LF based on the insertion characteristic 462. In some examples, the method 2000 includes actuating the proximal elements 610 toward the closed position to grasp the leaflet LF. The actuation captures the leaflet LF between the engagement surface 620 of the proximal elements 610 and the engagement surface 718 of the distal elements 702.

The above-described examples of a fixation device 500 may be utilized in a TEER procedure or any other tissue approximation procedures (e.g., procedures involving cardiac and non-cardiac tissues). A TEER procedure is a minimally invasive interventional procedure that aims to approximate cardiac valvular leaflets to reduce valvular regurgitation particularly in the mitral and triscupid valves. One of the challenges of this type of procedure is ensuring that the valve leaflets are adequately inserted into the fixation device, such as the fixation device 500, for example, so that when the fixation device is deployed, it remains secured to the tissue and sufficiently reduces regurgitation. Current TEER techniques typically rely on echocardiography and fluoroscopy for visualization which typically capture fixed views of the target valve.

Such visualization modalities, however, have inherent limitations that present challenges making leaflet capture and grasping one of, if not the most, challenging aspects of a TEER procedure. In particular, technique and technological limitations make it difficult to visually observe the leaflets within the fixation device during and after deployment so that the surgeon cannot directly observe how much leaflet is inserted into the fixation device or whether a leaflet has been captured at all. Thus, surgeons typically assess leaflet capture through circumstantial observations that are heavily reliant on surgeon experience. For instance, a surgeon might detect changes in leaflet movement and reductions in backflow using color Doppler to assess whether a leaflet has been captured and evaluate the effectiveness of the capture.

Difficulties in visualizing leaflet capture can lead to poor leaflet securement and, ultimately, single leaflet device attachment (SLDA), where only one leaflet is captured by the fixation device, or complete detachment (implant embolization). For instance, if a leaflet is not inserted far enough between the proximal and distal elements (i.e., shallow leaflet insertion), the elements may lack the necessary force to properly grasp and retain the tissue, potentially causing leaflet slippage and leading to SLDA or implant embolization. Therefore, it is generally insufficient to merely capture valve tissue between the proximal and distal elements. Instead, the capture must be of sufficient quality to ensure the fixation device remains securely attached to the leaflet. The quality of tissue capture can be influenced by several factors, one of which is the magnitude of the pinch force applied to the tissue.

Finite element analysis has been performed to quantify the pinch force within corresponding proximal and distal elements to help determine how deep a leaflet may be inserted to achieve an adequality high probability that a leaflet will be permanently captured with limited or otherwise acceptable risk of SLDA or embolization. The force distribution resulting from this analysis for a first clamp 4004 comprised of a distal element 702 and a proximal element 610 is shown in FIG. 4I and for a second, longer clamp 4004 comprised of the distal element 702 and the proximal element 610 is shown in FIG. 4J. As shown, the majority of the generated pinch force is applied to the tissue when approximately more than 50% of a maximum leaflet insertion depth is achieved.

Thus, leaflet security is ensured, and leaflet capture is sufficient when a leaflet LF is inserted between proximal and distal elements to a depth of more than 50% of a maximum insertion depth. The maximum insertion depth Lgrip, as depicted in FIGS. 4I and 4J, is the maximum length along which a leaflet LF can be captured between the proximal and distal elements, such as the proximal elements 702 and the distal elements 610. The maximum insertion depth Lgrip may be measured between a crotch 4005 (taking into account any fabric covering on the fixation device) formed between fixed ends 712, 713 and free ends 714, 715 of the respective distal elements 702 and the proximal elements 610. The crotch 4005 defines a closed end of the clamps 4004. The free ends 714, 715 define an open end of the clamps 4004. Although the distal and proximal elements 702, 610 typically capture tissue when each distal element 702 is at about 60 degrees relative to a longitudinal axis defined by a shaft 900 and are then moved to a final, closed position of 5 to 15 degrees (10 to 30 degrees relative each other), it should be noted that the pinch force generally does not change with the angle of the clamp 4004 up to about the initial capture angle of 60 degrees. Thus, the pinch force will be approximately the same throughout the range of motion from initial capture to the final, closed position.

FIG. 4K illustrates an optical sensing system 4100 according to an embodiment of the present disclosure. The optical sensing system 4100 generally includes a signal processing unit 4101, one or more optical sensors 4110, and an interface 4107 coupling the sensors 4110 to the signal processing unit 4101. The optical sensors 4110 each include an emitter 4112 and a detector 4114. The emitter 4112 includes a light source (e.g., a laser diode or light-emitting diode (“LED”)). The detector 4114 includes a detection element (e.g., a photodiode, phototransistor, charge-coupled device (“CCD”), complementary metal-oxide-semiconductor (“CMOS”), or photomultiplier tube (“PMT”)). As described below, the sensing system 4100 can be integrated into a medical device or system and may be configured to detect and measure various conditions within a patient during a medical procedure.

The optical sensing system 4100 may have a reflective configuration. In this configuration, the emitter 4112 directs light into an anatomical space within a patient.

The detector 4114 receives light reflected from the anatomical space and converts it into an electrical signal. This signal may then be processed by the signal processing unit 4101 to provide insights into the anatomical space.

In one implementation, the reflected light detected by the detector 4114 can be used by the system 4100 to determine the distance of one or more objects from the optical sensor 4110. The emitted light may pass through blood or other fluids within the anatomical space and interact with the object, which may be tissue and/or components of a medical device. When the light interacts with the object, portions of it may be absorbed, scattered, or reflected back, depending on the object's composition. By measuring the time delay between the emission and reception of the light and using the speed of light in the medium (e.g., blood), the system 4100 can calculate the distance of the object from the optical sensor 4110.

Additionally or alternatively, the reflected light captured by the detector 4114 may provide detailed information about tissue within the anatomical space. By analyzing characteristics of the reflected light, such as intensity, wavelength, and temporal variations, the system can differentiate between tissue types. Different biological tissues exhibit unique optical signatures due to variations in their structure and composition, which influence how they absorb, scatter, and reflect light.

For example, cardiac tissues such as leaflet tissue and chordae tendineae, for example, exhibit distinct optical behaviors. Leaflet tissue, being thinner and more elastic than chordae tendineae, tends to scatter and absorb light differently compared to the denser, more fibrous chordae. By emitting light at specific wavelengths, the system 4100 can distinguish between these tissue types.

Targeted wavelengths may be selected from the infrared (“IR”) spectrum, which spans a broad range of electromagnetic wavelengths, or from the near-infrared (“NIR”) spectrum, which is a subset of IR. These wavelengths are particularly effective at penetrating blood with minimal interference from water and hemoglobin absorption. In one example, wavelengths may be selected within the IR range of 500 to 1,000 nm. In another example, wavelengths may be selected within the NIR range of 700 to 1,500 nm, which overlaps with the lower portion of the IR spectrum. Additionally, the system 4100 may be configured to modulate wavelengths within these ranges so as to dynamically adjust the emitted light which may help further analyze tissue characteristics. Such modulation can enhance the ability of the system 4100 to identify subtle differences in tissue composition or structure.

In another example, the optical system 4100 may have a through-beam configuration. In this configuration, the emitter 4112 is positioned opposite the detector 4114. When tissue is positioned between the emitter 4112 and the detector 4114, the emitter 4112 emits light through the tissue which is then detected by the detector 4114.

In one implementation, the system 4100 may generate a binary signal indicating whether tissue is present between the emitter 4112 and the detector 4114.

When the emitted light is attenuated as it passes through the tissue, the system 4100 interprets this reduction in light intensity and outputs a binary signal to indicate the presence of tissue. Conversely, when no attenuation occurs, the system 4100 signals the absence of tissue. Thus, in the through-beam configuration, the system 4100 may provide a binary output (i.e., tissue present or tissue absent) at the sensor's location.

Additionally or alternatively, in the through-beam configuration, the system 4100 may analyze the attenuation of light as it passes through the tissue to gather additional information about the tissue. For example, the system 4100 may be configured to determine the thickness of the tissue based on the degree of light absorption and scattering. Furthermore, the type of tissue can be identified by examining the spectral characteristics of the transmitted light, as described above with respect to the reflective configuration.

Signals generated by the sensors 4110 can be processed and converted into a readable output signal by the signal processing unit 4101, enabling a surgeon to assess conditions within the patient. As shown in FIG. 4K, the signal processing unit 4101 generally includes a power source 4103 and a signal processing module 4105.

The power source 4103 is configured to provide electrical energy to the system 4100, including any circuits that drive the optical sensors 4110 or process the generated signals. The power source 4103 may be a DC power source, such as a battery or an external DC power adapter, for example. However, in some applications, an AC power source may be utilized, with AC to DC conversion implemented within the system 4100 for signal processing and sensor operation.

The signal processing module 4105 is configured to receive and process signals generated by the optical sensors 4110. In this regard, the signal processing module 4105 handles one or more tasks, such as signal amplification, noise reduction, wavelength filtering, and/or analog-to-digital conversion, for example. For instance, the signal processing module 4105 may include one or more amplifier circuits (e.g., transimpedance amplifiers) to boost low-current signals from the detector 4114 of the optical sensor 4110 to measurable voltage levels. The signal processing module 4105 may also include one or more filter circuits (e.g., low-pass, high-pass, and band-pass filters) to reduce electronic noise from the signals and, in some instances, to isolate specific wavelength ranges of the detected light. Further, the signal processing module 4105 may include an analog-to-digital converter (“ADC”) to transform analog optical signals into digital signals suitable for further processing by a processor. Thus, in some configurations, the signal processing module 4105 may include a processor and a memory to perform additional signal processing tasks, such as spectral analysis and/or the application of machine learning algorithms for classifying optical signal patterns, for example.

The processed signals from the signal processing module 4105 may be output to an output device, such as an output device 4102 shown in FIG. 4K. The output device 4102 may be a display (e.g., a monitor, touchscreen, projector, or television) or another visual indicator (e.g., LED array) that provides visual feedback to assist in a surgeon's decision-making process. The results may be presented as raw data, such as light intensity readings or wavelength measurements, or as processed interpretations of the signals generated by one or more optical sensors 1010, simplifying the decision-making process for the surgeon.

The interface 4107 is configured to communicatively coupled the sensors 4107 and the signal processing unit 4101. The interface 4107 may be a hardwired interface or a wireless interface, for example. In a hardwired configuration, the interface 4107 may include one or more conductors, which may carry electrical signals between the sensors 4110 and the signal processing unit 4101. Such configuration enables stable and reliable signal transmission with minimal noise interference. In a wireless configuration, the interface 4107 may additionally or alternatively include transmitters, receivers, and/or transceivers which may be enabled with any one of various wireless protocols (e.g., Bluetooth Low Energy (“BLE”), Near Field Communication (“NFC”), or Zigbee) to transmit signals from the sensors 4110 to the signal processing unit 4101.

FIG. 4L illustrates an exemplary implementation of the optical sensing system 4100 in which one or more sensors 4110 of the sensing system 4100 are coupled to the fixation device 500 in a reflective configuration. The fixation device 500 includes a first clamp 4004a and a second clamp 4004b each comprised of the distal element 702 and the proximal element 610. The fixation device 500 may also include a center portion 4060 disposed between each clamp 4004a, 4004b. When the center portion 4060 is provided, the first clamp 4004a extends outwardly relative to the center portion 4060 for capturing a first tissue (e.g., a first valve leaflet), and the second clamp 4004b extends outwardly relative to the center portion 4060 for capturing a second tissue (e.g., a second valve leaflet). In this regard, the clamp 4004a, 4004b defines an open end 4001 and a closed end 4002. The closed end 4002 is positioned closest to the center portion 4060. When tissue is captured between one of the clamps 4004a, 4004b, the tissue enters into the clamp 4004a, 4004b from the open end 4001 and moves toward the closed end 4002.

The components of the fixation device 500 may be any of the components described above with respect to the fixation device 500. In this regard, the distal elements 702 may be any of the aforementioned distal elements (e.g., the distal elements 702) and may be operable via an actuation mechanism. The proximal elements 610 may be any of the aforementioned proximal elements (e.g., the proximal elements 610). Additionally, the center portion 4060 may include a coupling member (e.g., a coupling member 560) which is configured to releasably coupled to a catheter 900. In another example, the center portion 4060 may include a base (e.g., a base 532) and/or a stud (e.g., a stud 540) which may be coupled to the distal elements 702 and may be configured to releasably coupled to an actuator rod (e.g., an actuator rod 810) for actuation of the distal elements 702. In a further example, the center portion 4060 may further include a locking mechanism (e.g., a locking mechanism 1000) configured to secure the distal elements 702 in a selected position. The fixation device 500 may also include a cover like that of the cover 560 (see also a cover 560 of FIG. 4W).

In the example depicted in FIG. 4L, the sensors 4110 are arranged in a reflective configuration. The optical sensor 4110 is positioned between the proximal element 610 and the distal element 702 of the clamp 4004a, 4004b. The optical sensor 4110 is positioned at or proximate to the closed end 4002 of the respective clamp 4004a, 4004b such that the emitter 4112 and the detector 4114 thereof are directed towards the open end 4001. In other words, the emitter 4112 is configured to emit light into a space between the proximal element 610 and the distal element 702 toward the open end 4001, and the detector 4114 is configured to detect light returning from this space toward the closed end 4002. As depicted, the emitter 4112 and the detector 4112 are in a stacked relationship. The detector 4114 is also shown as being closest to the proximal element 610. However, in other examples, the emitter 4112 may be closest to the proximal element 610. Additionally, in other examples, the emitter 4112 and the detector 4114 may not be stacked but may rather be positioned in a side-by-side arrangement.

In one implementation of the sensors 4110 with the fixation device 500, each sensor 4110 is secured to the closed end of a clamp 4004, such as via adhesive, one or more magnets, an interference fit, flexible PCB circuit board or the like. For example, the proximal element 610 may have a fixed end 632 coupled to a base 4050. The base 4050 partially defines the closed end 4002 of the clamp 4004a, 4004b. The sensor 4110 may be coupled to the base 4050 via adhesive or the like. Alternatively, the base 4050 may include a bracket or housing onto which the sensor 4110 is mounted via magnets or an interference fit, for example. In other examples, the distal element 702 at least partially defines the closed end 4002. The sensor 4110 may be coupled to the distal element 702, such as on an engagement surface thereof. In such configuration, the sensor 4110 may move with the distal element 702.

In another implementation of the sensors 4110 (schematically represented) with the fixation device 500, each sensor 4110 may be free floating with respect to the closed end 4002 of the clamp 4004a, 4004b. For example, the emitter 4112 and the detector 4114 may project from the center portion 4060 of the fixation device 500 through an opening, such as an opening within a base 702, and into the space between the proximal element 610 and the distal element 702. Thus, the emitter 4112 and the detector 4114 may be stationary relative to the movements of the proximal and distal element 610, 702.

In the operation of the fixation device 500, light is emitted from the emitter 4112 into the space between the proximal element 610 and the distal element 702. Such light may be in the IR or NIR spectrum, as described above. The clamp 4004a, 4004b is moved to an open position at a target tissue (e.g., mitral valve or tricuspid valve). A first tissue, such as a first leaflet LF1, enters into the open end 4001 of the first clamp 4004a, and a second tissue, such as a second leaflet LF2, enters the open end 4001 of the second clamp 4004b. As leaflets LF1 and LF2 enter into their respective clamps 4004a, 4004b, the light emitted from the emitter 4112 interacts with the tissue such that some light is absorbed and some light is deflected back towards the detector 4114. The detector 4114 detects the reflected light and produces a corresponding signal which is communicated back to the signal processing unit 4101 where it is processed.

Such signal may be indicative of the distance of leaflet LF1, LF2 relative to the sensor 4110. Since the position of the sensor 4110 within the clamp 4004a, 4004b is known, the position of tissue with respect to the clamp 4004a, 4004b can also be determined. For example, as described above, it is desirable for tissue to be grasped between the clamp 4004a, 4004b with the tissue positioned at a depth greater than 50% of the grip length of the clamp 4004a, 4004b as measured from the open end 4001. The signal processing unit 4101 may generate output signals to the output device 4102 to inform the surgeon of the depth of the tissue between the clamp 4004a, 4004b and whether the tissue has reached this depth. If the tissue has not reached this depth, the surgeon may attempt a regrasp.

The signal generated by the detector 4114 may also be indicative of the type of tissue within the clamp 4004a, 4004b. For example, if chordae tendineae of the valve is inadvertently captured between the clamp 4004a, 4004b, such signal may indicate that chordae is within the clamp 4004a, 4004b, and the signal processing unit 4101 may inform the surgeon who then may attempt a regrasp to and unsnag the chordae. In another example, the signal may indicate that leaflet tissue is between the clamp 4004a, 4004b and, as mentioned above, the depth may also be determined allowing the surgeon to assess the quality of the grasp and whether a regrasp should be attempted.

FIG. 4M depicts an exemplary optical sensor 4010 according to an example of the fixation device 500. In this configuration, the emitter 4112 includes a light source 4012. The light source 4012 may be an LED or a laser diode configured to emit light in the IR or NIR spectrum, for example. The emitter 4112 may also include an optical element 4014. The optical element 4014 may be a beam splitter or the like for separating the light produced by the light source 4012 into one or more beams. Alternatively, the emitter 4112 may have a plurality of the light sources 4012 (e.g., laser diodes) for producing separate beams of light. The emitter 4112 may also be oriented at an angle toward the proximal element 610, such as when the proximal element 610 is in a lowered position, light emitted from the emitter 4112 is projected onto the proximal element 610. In this regard, the emitter 4112 is configured to project one or more beams of light at a one or more locations along a grip length Lgrip of the proximal element 610. For example, the emitter 4112 may be configured to project a first beam b1, a second beam b2, and a third beam b3 onto separate locations of the proximal element 610. The first beam bl may be projected to a location beyond 50% of the grip length of the proximal element 610 as measured from a free end 632 thereof. The second beam b2 may be projected to a location at a midline of (i.e., at 50% the grip length) of the proximal element 610. The third beam b3 may be projected at a location between the free end 632 and the midline (e.g., between 25% and 50% of grip length).

As shown, light reflected back from the beam b1-b3 is detected by the detector 4114. The detector 4114 includes a detection element 4011. The detection element 4011 may be a photodiode (e.g., silicon photodiode or avalanche photodiode), phototransistor, CCD, CMOS, or PMT, for example. The detector 4114 may also include an optical element 4013. The optical element 4013 may be a lens or the like for capturing the reflected light and concentrating it on the detector element 4011. The distance between each point at which the beams b1-b3 intersects the proximal element 610 and the detector 4114 may be determined by the signals generated by the detector element 4011. For example, a distance dl is a distance between the detector 4114 and the point at which the beam bl intersects the proximal element 610, a distance d2 is a distance between the detector 4114 and the point at which the beam b2 intersects the proximal element 610, and a distance d3 is a distance between the detector 4114 and the point at which the beam b3 intersects the proximal element 610. Such distances may be known to the system 4100 such that when no tissue is positioned between the proximal element 610 and the distal element 702, the system 4100 may be in a first state in which a variable response of the detector 4114 confirms tissue is absent. In some examples, the beam b1-b3 may be configured to have different wavelengths or intensities which may help the detector 4114 distinguish between the beam b1-b3.

As illustrated in FIG. 4N, when tissue, such as leaflet LF1, is captured between the proximal element 610 and the distal element 702, the tissue LF1 may intersect one or more of the beams b1-b3. Interruption in any one of these beam paths to the proximal element 610 indicates the presence of the tissue LF1 at least at the location in which the beam b1-b3 would intersect the proximal element 610. For example, where the leaflet tissue LF1 intersects the third beam b3, the light of the beam b3 reflected back to the detector 4114 is disrupted and the system 4100 enters into a second state in which the detector 4114 confirms the variable response is proportional to tissue capture at the projected position of the third beam b3. Such disruption may be sensed by the detector 4114 by light reflected from the tissue LF1 which may have a distance d3′ different than the distance d3. Additionally, the light intensity from the third beam b3 deflected off of the tissue LF1 may be reduced further confirming the presence of the tissue LF1. In other words, the detector 4114 is configured to generate a signal based on the deflected light.

Such signal may be a dynamic signal that changes over time based on the optical interactions between the light beams (e.g., the beams b1-b3) generated by the detector 4114 and the proximal element 610 and/or tissue (e.g., LF1, LF2). Such dynamic signal may indicate the interaction of tissue at any one or more of the beams b1-b3 which in turns may be used by the system 4100 to determine the location of tissue within the clamp 4004a, 4004b. Additionally, the dynamic signal may further indicate the type of tissue interacting with the beams b1-b3.

Although FIGS. 4M and 4N depict a sensor 4210 projecting light onto the proximal element 610, it should be understood that a sensor 4112, 4114 may be configured to alternatively project light onto the distal element 610 in a similar manner.

FIG. 40 depicts another exemplary optical sensor 4210 (schematically represented) according to an example of the fixation device 500. In this configuration, the emitter 4112 includes a light source 4212. The light source 4212 may be an LED or a laser diode configured to emit light in the IR or NIR spectrum, for example. The emitter 4112 may also include an optical element 4214. The optical element 4214 may be a diffuser or the like for diffusing the light produced by the light source 4212 into a cone of light which extends into the space between the proximal element 702 and the distal element 610. In one example, the emitter 4112 and the detector 4114 may be oriented generally parallel to the distal element 702 such that the cone of light extends toward the open end 4001 of the clamp 4004a.

When the leaflet tissue LF1 is captured between the proximal element 610 and the distal element 702, light from the emitter 4112 is at least partially deflected and at least partially absorbed by the tissue LF1. The reflected light is detected by the detector 4114. The detector 4114 includes a detection element 4211. The detection element 4211 may be a photodiode (e.g., silicon photodiode or avalanche photodiode), phototransistor, CCD, CMOS, or PMT, for example. The detector 4114 may also include an optical element 4213. The optical element 4213 may be a lens or the like for capturing the reflected light and concentrating it on the detector element 4211. The detector is configured to generate a signal based on the deflected light. Such signal may indicate a distance between the sensor and the tissue LF1 and correspondingly a depth of the tissue LF1 within the clamp. Additionally, such signal may be indicative of the type of tissue disposed between the proximal element 702 and the distal element 610, as described above.

FIG. 4P depicts another exemplary optical sensor 4310 (schematically represented) according to an example of the fixation device 500. In this configuration, the emitter 4112 includes a light source 4312. The light source 4312 may be an LED or a laser diode configured to emit light in the IR or NIR spectrum, for example. The emitter 4112 may also include an optical element 4314. The optical element 4314 may be a lens or the like for producing a concentrated beam of light from the light source 4312. Such beam is projected into the space between the proximal element 702 and the distal element 610. In one example, such as that depicted in FIG. 4P, the emitter 4112 may be oriented parallel to the distal element 702, while the detector 4114 may be oriented at an angle relative to the emitter 4112 and toward the beam of light.

When the leaflet tissue LF1 is captured between the proximal element 610 and the distal element 702, light from the emitter 4112 is at least partially deflected and at least partially absorbed by the tissue LF1. The reflected light is detected by the detector 4114. The detector 4114 includes a detection element 4311. The detection element 4311 may be a multipart photodiode, such as a two-part photodiode having a near component N and a far component F. The detector 4114 may also include an optical element 4314.

The optical element 4314 may be a lens, beam splitter, prism, diffuser, or the like for capturing the reflected light directing it at the near portion N, the far portion F, or between the near and far portion of the detection element 4311.

The detector 4114 produces a signal based on the deflected light and where the deflected light is concentrated in the two-part photodiode 4311. For example, when the leaflet tissue LF1 is located at a first position P1, such as beyond 50% of a grip length Lgrip of the proximal element 610, the reflected light may be concentrated on the near portion N of the detector element 4311 indicating that leaflet tissue is in the first position P1. In another example, when the leaflet tissue LF1 is located at a second position P2, such as at a midline of the proximal element 632, the reflected light from the tissue LF2 may be concentrated at a location between the near portion N and the far portion F of the detector element 4311 to indicate that the tissue in the second position P2. In a further example, when the leaflet tissue LF1 is located at a third position P3, such as between the free end 632 of a gripper 610 and the midline, the reflected light from the tissue LF1 may be concentrated on the far part F of the detector element 4114 indicating that the tissue LF1 in the third position P3. The signals generated by the detector element 4311 may also be indicative of the type of tissue disposed between the proximal element 702 and the distal element 610, as described above.

FIG. 4Q illustrates another exemplary implementation of the optical sensing system 4100. In this implementation, one or more sensors 4310 (schematically represented) of the sensing system 4100 are coupled to a fixation device 500 in a through-beam configuration. The fixation device 500 is like the fixation device 500 in that it includes a first and second clamp 4004a, 4004b each comprised of the distal element 702 and the proximal element 610. Additionally, the fixation device 500 may also include a center portion 4060 disposed between the clamp 4004a, 4004b. Also, like the fixation device 500, each of the components of the fixation device 500 (e.g., the distal elements 702, the proximal elements 610, and the center portion 4060) may be any of the components described above with respect to the fixation device 500.

As illustrated in FIG. 4Q, the sensors 4310 are coupled to the proximal elements 610 and the distal elements 702 and each include the emitter 4112 and the detector 4114. The emitter 4112 includes a light source (e.g., a laser diode or LED) which is configured to emit light in the IR or NIR spectrum. The detector 4114 includes a detection element (e.g., a photodiode, phototransistor, CCD, CMOS, or PMT) which is configured to detect the light emitted from the emitter 4112. In the depicted example, the emitter 4112 is coupled to the distal element 702, and the detector 4114 is coupled to the proximal element 610. However, in another example, the detector 4114 may be coupled to the distal element 702, and the emitter 4112 may be coupled to the proximal element 610. The emitter 4112 and the detector 4114 are disposed opposite each other such that when the distal elements 702 are in an open position (e.g., 120 degrees relative to each other) and the proximal elements 610 are in a lowered position, the emitter 4112 and the detector 4114 are disposed directly opposite each other allowing light emitted from the emitter 4112 to be directed to the detector 4114.

In the operation of the fixation device 500, the distal elements 702 are moved to the open position, and the proximal elements 610 are moved to the lowered position (as shown in the right side of FIG. 4Q). When tissue is absent from the clamp 4004a, 4004b or the tissue LF1, LF2 has not reached the location of the emitter 4112 and the detector 4114, light emitted from the emitter 4112 passes through the space between the distal and proximal elements 702, 610 to the detector 4114 relatively undisturbed such that a signal generated by the detector 4114 indicates the absence of tissue. Conversely, when the tissue LF1, LF2 reaches the location of the emitter 4112 and the detector 4114 and is disposed therebetween, as shown with respect to the first clamp 4004a of FIG. 4Q, light emitted from the emitter 4112 passes through the tissue LF1 which is then detected by the detector 4114. The reduction in light intensity indicates the presence of the tissue LF1. Additionally, the detector 4114 may generate a signal based on the degree of light absorption and scattering which can be interpreted by the signal processing unit 4101 to determine a thickness of the tissue and the type of tissue grasped between the clamp 4004a. As such, the sensor 4310 may be utilized to distinguish between leaflet tissue and chordae tendineae, for example, Referring now in addition to FIG. 4R, which depicts a proximal element 610 according to an example of the fixation device 500. The proximal element 610 is similar to the proximal element 610, and accordingly, like elements are assigned like reference numerals but within a larger number series. The proximal element 610 includes an elongate body 4141 and a plurality of frictional elements 620. The elongate body 4141 defines a gripping length Lgrip extending from a first end 632 (or fixed end) to a second end 634 (or free end). In the example depicted, the detector 4114 (or the emitter 4112) is coupled to the elongate body 4141. For example, an opening 636 may extend through the elongate body 4141, and the detector 4114 may be at least partially positioned within the opening 636 such that the detector 4114 faces a corresponding distal element 702.

Additionally, the detector 4114 is positioned at a midline ML of the proximal element 610. In other words, the detector 4114 is positioned at 50% of the grip length Lgrip of a proximal element 2610 as measured from free end 634. Thus, when the detector 4114 signals the presence of tissue, the tissue is determined to extend to at least 50% of the total grip length Lgrip.

Referring now in addition to FIG. 4S, which depicts a proximal element 610 according to an example of the fixation device 500. The proximal element 610 is similar to the proximal element 610, and accordingly, like elements are assigned like reference numerals but within a larger number series. In this example, the detector 4114 (or the emitter 4114) is coupled to the elongate body 4141, such as within an opening 2116 thereof. Additionally, the detector 4114 is positioned at a location between a midline ML and fixed end 32 of the proximal element 610. In other words, the detector 4114 is positioned at a location beyond 50% of the grip length Lgrip of the proximal element 610 as measured from free end 634. Thus, when the detector 4114 signals the presence of tissue, the tissue is determined to extend beyond 50% of the grip length Lgrip, which may further indicate regrasping is not needed as the tissue may be considered sufficiently deep within the clamp 4004a, 4004b to limit the risk of SLDA.

Referring now in addition to FIG. 4T, which depicts a distal element 702 according to an example of the fixation device 500. In this example, the emitter 4112 (or the detector 4114) is coupled to the distal element 702, such as within a window 4128 thereof. The window 4128 may extend through an engagement surface 718 of the distal element 702. In some examples, a substrate 4415, such as a silicon substrate, may be disposed within the window 4128 and the emitter 4112 may be connected to the substrate 4415 to secure it to the distal element 702. Additionally, the emitter 4112 is positioned at a location corresponding to the detector location on a corresponding proximal element.

Thus, in one example, the emitter 4112 may be located at a midline ML of the distal element 702 which corresponds to the placement of the detector 4114 on the proximal element 610. In another example, the emitter 4112 may be located at a location beyond the midline ML corresponding to the placement of the detector 4114 on the proximal element 610.

Referring now in addition to FIG. 4U, which depicts a proximal element 610 according to an example of the fixation device 500. The proximal element 610 is similar to the proximal element 610, and accordingly, like elements are assigned like reference numerals but within a larger number series. In this regard, the proximal element 610 is coupled to an arm bend feature 4260 which is coupled to a base 4250. The base 4250 may be coupled to a corresponding distal element (e.g., the distal element 702). Additionally, the proximal element 610 includes first and second side portions 4247 (or elongate members) which define a window 4248 therebetween. The detector 4114 (or the emitter 4112) is coupled to the proximal element 610, such as at a midline ML thereof or a location between midline ML and a fixed end 632. In some examples, a substrate 4515, such as a silicon substrate, may be disposed within the window 4248 and the emitter 4112 may be connected to the substrate 4515 to secure it to the distal element 610.

Additionally, the emitter 4112 (or the detector 4114) may be positioned on the base 4250 at a location corresponding to the location of the detector 4114 on the proximal element 610.

According to the foregoing examples, the optical sensing system 4100 includes the signal processing unit 4101, one or more sensors 4110, and an interface 4107 between the sensors 4110 and the signal processing unit 4101. Thus, reference to the sensors 4110 in the following discussion includes any of such optical sensors. Further, as mentioned above, the system 4100 may include the output device 4102 for presenting discernable information from the signal processing unit 4101, aiding in surgeon decision-making.

In one exemplary implementation of the optical sensing system 4100, the system 4100 may be integrated into a fixation device, such as the fixation device 500. In this regard, the sensors 4110 may be coupled to one of these fixation devices as described above, Additionally, the signal processing unit 4101 may also be coupled to any one of these fixation devices 500, such as to a center portion 4060 thereof. In this example, the signal processing unit 4101 may process signals from each sensor 4110 and then transmit an output signal to the output device 4102 via a wireless protocol or via a hardwired connection through a delivery catheter (e.g., delivery catheter 850), for example.

In another exemplary implementation of the optical sensing system 4100, the sensors 4110 may be coupled to a fixation device (e.g., the fixation device 500) as described above and may communicate with the signal processing unit 4101 positioned external to the patient. Thus, in this example, a transmitter may be coupled to center portion (e.g., the center portion 4060) and may be configured to wirelessly transmit signals generated by the sensors 4110 to an extracorporeally located signal processing unit 4101.

In a further exemplary implementation of the optical sensing system 4100, the system 4100 may be integrated with a medical system, such as an interventional system 1100. The interventional system 1100 includes a fixation device 500 and a delivery system 4600. The fixation device 500 may be any of the fixation device 500. The delivery system 4600 generally includes a delivery device handle 1210 and delivery catheter 1230 extending from the handle 1210. The delivery catheter 1230 is representative of delivery catheters 850. The delivery device handle 1210 includes a plurality of controls 1400, 1300, 1500 for controlling the fixation device 500, such as the proximal elements 610 and the distal elements 702, and for disconnecting the fixation device 500 from a shaft 900 of the catheter 1230 so as to leave the fixation device 500 within a patient as an implant.

In one example, the sensors 4110 may be coupled to the fixation device 500 in accordance with any of the foregoing examples described above. Additionally, the signal processing unit 4101 may be coupled to the delivery device handle 1210, such as embedded therein or separately connected (e.g., via a cable). In this example, the interface 4107 between the signal processing unit 4101 and the sensors 4110 may be a hardwired interface which may extend from implant 500, through the catheter 1230 to catheter handle 1210. For example, gripper lines 804 may be comprised of insulating wiring which may extend through the catheter 1230 and have the dual functions of providing an electrical connection between the sensors 4110 and control unit 4101 and raising and lowering the proximal elements 610. Alternatively, electric conductors (e.g., leads or traces) may extend through the catheter 1230 and into a center portion 4360 of the fixation device 500. When the fixation device 500 is disconnected from the delivery catheter 1230 during a procedure, the interface 4107 between the sensors 4110 and control unit 4101 may be disconnected and the sensors 4110 may be left within the fixation device 500 after disconnection between the catheter 1230 and the fixation device 500.

As mentioned, the signal processing unit 4101 may be disposed in the delivery device handle 1210. In this example, the signal processing unit 4101 may be coupled via a cable or otherwise to other external devices, such as the output device 4102. In another example, the delivery device handle 1210 may include the output device 4102. For example such output device may be an LED array 4355 that turns on or off when any one of the sensors 4110 indicate leaflet tissue has reached the designated location within the fixation device 500.

In another example, the signal processing unit 4101 may be positioned external to a delivery system 1200. In such example, the signal processing unit 4101 may be coupled to hardwiring within the delivery system 4600 via a cable or otherwise so as to electrically couple the signal processing unit with the sensors 4110.

FIG. 4X depicts an optical sensing system 4600 according to an embodiment of the present disclosure. The optical sensing system 4600 is similar to the optical sensing system 4100 in that it includes a signal processing unit 4601 and one or more sensors 4610 that each include the emitter 4112 and the detector 4114. The emitter 4112 includes a light source (e.g., a laser diode or LED) which is configured to emit light in the IR or NIR spectrum. The detector 4114 includes a detection element (e.g., a photodiode, phototransistor, CCD, CMOS, or PMT) which is configured to detect the light emitted from the emitter 4112. However, unlike the optical sensing system 4100, the emitter 4112 and the detector 4114 may be directly coupled to the signal processing unit 4601, as shown. Additionally, the system 4600 includes an optical fiber bundle 4620 that includes an emitter optical fiber 4622 that is coupled to the emitter 4112, and a detector optical fiber 4624 that is coupled to the detector 4114. The optical fibers 4624, 4622 may be plastic optical fibers or glass optical fibers.

The signal processing unit 4601 and the sensors 4610 may be disposed within a delivery system handle, such as a delivery system handle 4600, for example. As illustrated in FIG. 4X, optical fibers 4620 may extend into the delivery catheter 850 toward a fixation device, examples of which are described below.

FIG. 4Y illustrates an exemplary implementation of the optical sensing system 4600 which deploys optical fibers 4630, 4632 in a reflective configuration. In this implementation, optical fiber bundles, such as first and second optical fiber bundles 4620, extend through a catheter 4512 to the fixation device 500. The fixation device 500 is like the fixation device 500 in that it includes a first and second clamp 4004a, 4004b each comprised of the distal element 702 and the proximal element 610. Additionally, the fixation device 500 may also include a center portion 4560 disposed between the clamp 4004a, 4004b. Also, like the fixation device 500, each of the components of the fixation device 500 (e.g., the distal elements 702, the proximal elements 610, and the center portion 4560) may be any of the components described above with respect to the fixation device 500.

As shown in FIG. 4Y, optical fiber bundles 4620 may extend through catheter 4512, a catheter shaft 4511, and into a center portion 4560 of the fixation device 500.

Emitter and detector fibers 4630, 4632 of each bundle 4620 extend from the center portion 4560 and into a space between each respective clamp 4004a, 4004b. Emitter and detector fibers 4630, 4632 may be docked within a corresponding mount 4640, 4642 (or dock) which holds them in place and directs respective ends of fibers 4630, 4632 in a direction toward the space between the clamps 4004a, 4004b such that light is emitted from emitter fiber 4630 into the space, and light reflected from the space enters detector fiber 4632. In this regard, mounts 4630, 4632 are positioned at a closed end of each clamp 4004a, 4004b, and fibers 4630, 4632 are arranged to detect the presence of tissue within the clamps 4004a, 4004b and to measure characteristics of the tissue in a manner similar to that described above with respect to the fixation device 500 of FIG. 4L.

FIG. 4Z illustrates another exemplary implementation of the optical sensing system 4600 which deploys optical fibers 4630, 4632 in a through-beam configuration. In this implementation, optical fiber bundles, such as first and second optical fiber bundles 4620a, 4620b, extend through a catheter 4610 to the fixation device 500. The fixation device 500 is like the fixation device 500 in that it includes a first and second clamp 4004a, 4004b each comprised of the distal element 702 and the proximal element 610.

Additionally, the fixation device 500 may also include a center portion 4660 disposed between the clamp 4004a, 4004b. Also, like the fixation device 500, each of the components of the fixation device 500 (e.g., the distal elements 702, the proximal elements 610, and the center portion 4660) may be any of the components described above with respect to the fixation device 500.

As shown in FIG. 4Z, optical fiber bundles 4620 may extend through a catheter 4612, a catheter shaft 4611, and into a center portion 4660 of the fixation device 500. Emitter and detector fibers 4630, 4632 of each bundle 4620 extend from the center portion 4660 and to respective proximal elements 610 and distal elements 702. Emitter and detector fibers 4630, 4632 may be docked within a corresponding mount 4640, 4642 (or dock) which holds them in place. Such mounts 4640, 4642 are respectively disposed on the proximal and distal elements 610, 702 directly opposite each other such that, when fibers 4630, 4632 are docked thereto, ends of fibers 4630, 4632 are directed toward each other. Thus, when tissue is positioned therebetween, light emitted from emitter fiber 4630 passes through tissue to detector fiber 4620 to detect the presence of tissue within the clamps 4004a, 4004b and to measure characteristics of the tissue in a manner similar to that described above with respect to the fixation device 500 of FIG. 4Q.

FIG. 4AA-4AC illustrates a further exemplary implementation of the optical sensing system 4600. In this implementation, optical fiber bundles, such as first and second optical fiber bundles 4620a, 4620b, extend through a catheter 900 towards the fixation device 500. The fixation device 500 is like the fixation device 500 in that it includes a first and second clamp 4004a, 4004b each comprised of the distal element 702 and the proximal element 610. Additionally, the fixation device 500 may also include a center portion disposed between the clamp 4004a, 4004b. Also, like the fixation device 500, each of the components of the fixation device 500 (e.g., the distal elements 702, the proximal elements 610, and the center portion) may be any of the components described above with respect to the fixation device 500.

As shown in FIG. 4AA and 4AB, fiber bundles 4620a, 4620b extend through delivery catheter and terminate at an end of delivery catheter such that ends of emitter and detector fibers 4630, 4632 are exposed at the end of delivery catheter and point toward the fixation device 500. As such, light emitted from emitter fibers 4630 is directed toward a central region of the fixation device 500, and light reflected back from tissue captured within the fixation device 500 is detected by detector fibers 4632.

As shown in FIG. 4AC, catheter 500 has a plurality of lumens 4715 extending through it. The lumens 4715 are circumferentially arranged about catheter shaft 900. In one example, the catheter 4710 includes eight lumens 4715. However, in other examples, the catheter 4710 may have less than eight lumens 4715 (e.g., six lumens 4715) or more than eight lumens 4715 (e.g., nine lumens 4715) circumferentially arranged about a catheter shaft 4710. A first plane P1 and a second plane P2 bisect a catheter 4710 with first and second planes P1, P2 being arranged orthogonal to each other. Second plane P2 intersects the clamps 4004a, 4004b, and first plane P1 extends equidistantly between the clamps 4004a, 4004b. First and second fiber bundles 4620a, 4620b may extend through separate lumens 4715. In one example, first and second fiber bundles 4620a, 4620b extend through lumens 4715 disposed opposite each other. Additionally, first and second fiber bundles 4620a, 4620b may extend through lumens 4715 that are circumferentially offset relative to both first and second planes P1, P2. For example, first and second fiber bundles 4620a, 4620b may extend through lumens that are offset from first plane P1 by angle that is 30 degrees to 45 degrees relative to first plane P1. Such angular offset allows light emitted from emitter fiber 4630 to avoid interacting with the proximal element 610 and to instead interact with tissue captured by the clamps 4004a, 4004b.

In operation, when it is believed that tissue has been captured by the fixation device 500, the distal elements 702 are closed into a position adjacent to coupling member 4760, as illustrated in FIG. 4AB. For example, the distal elements 702 may be closed to an angle of 10 to 30 degrees relative to each other. In this closed position, captured tissue is positioned adjacent to a center portion 4760 of the fixation device 500 and at least partially circumferentially extends about the center portion 4760. Light emitted from emitter fibers 4630 intersects the tissue within each clamp 4004a, 4004b and is at least partially reflected back to detector fibers 4632 which directs the light to the detector 4114 thereby generating as signal indicative of one or more characteristics of the tissue. For example, light reflected from the tissue may measure the distance of tissue from emitter and detector fibers 4630, 4632 which can in turn determine how much tissue is engaged by the clamps 4004a, 4004b. Additionally, at least due to the circumferential positioning of fiber bundles 4620a, 4620b, the reflected light can determine the level of tissue engagement circumferentially about the center portion 4760 which is also indictive of the amount of tissue grasped by the proximal elements 610. For instance, shallow tissue insertion results in limited circumferential extension, while deep tissue insertion results in greater circumferential extension of tissue about the center portion 4760. Thus, in some examples, additional fiber bundles 4620 may extend through additional lumens 4715 to obtain a higher resolution of the circumferential extension of the tissue.

FIG. 4AD-4AF illustrate a further exemplary implementation of the optical sensing system 4600. In this implementation, rather than coupling emitter and detector components to a fixation device emitter and detector components are coupled to a ring 4130 disposed at a distal end of a delivery catheter shaft 4010 and directed toward the fixation device 500. Locating the emitter and detector components on the ring 4130 rather than on the fixation device 500 may reduce complexity as it facilitates routing of optical fibers, electrical leads, or both from the emitter and detector components through the delivery catheter shaft 4010 to a signal processing unit, such as the signal processing unit 4101. The fixation device 500 is like the fixation device 500 in that it includes a first and second clamp 4004a, 4004b comprising, respectively, a first distal element 702a and a first proximal element 610a, and a second distal element 702b and a second proximal element 610b. Additionally, the fixation device 500 may also include a coupling member 540 disposed between the clamp 4004a, 4004b. Also, like the fixation device 500, each of the components of the fixation device 500 (e.g., the distal elements 702a, 702b, the proximal elements 610a, 610b, and the coupling member 540) may be any of the components described above with respect to the fixation device 500.

Referring to FIG. 4AD and 4AE, the ring 4130 may be disposed at a distal end of delivery catheter, such as delivery catheter 1230, and may be releasably coupled to the fixation device 500. The ring 4130 may be a radio-opaque ring, also referred to herein as an RO ring, and may be made from a stainless-steel material, such as 304 SS, 316L, or 316LVM. As shown in FIG. 4AE, the ring 4130 may include a head 4034, a body 4032 extending proximally from the head 4034, and a shaft 4036 extending proximally from the body 4032. The head 4034 may have a larger diameter than the body 4032, and the body 4032 may have a larger diameter than the shaft 4036. The shaft 4036 and the body 4032 may be configured to couple to the delivery catheter. However, other configurations for coupling to the delivery catheter are contemplated. For example, in some embodiments, the shaft 4036 may not be included, and the body 4032 may be configured as the only feature that couples directly to the delivery catheter. The head 4034 may have an outer diameter sized to facilitate retraction of the fixation device 500 into a steerable guide catheter. In one example, the outer diameter of the head 4034 may be about 0.004 inches larger than a diameter of the guide catheter, such that the ring 4130 may guide the fixation device 500 during retraction. In one example, the outer diameter of the head 4034 may be about 0.212 inches.

The ring 4130 may be connected to the delivery catheter shaft 4010 such that the ring 4130 rotates with the delivery catheter shaft 4010. In this configuration, a sensor package disposed on the ring 4130 may maintain a fixed rotational alignment with first and second clamps 4004a, 4004b of the fixation device 500 during rotation of the delivery catheter shaft 4010.

As shown in FIG. 4AE and 4AF, the head 4034 may be configured to receive a sensor package. In this regard, the head 4034 may include a cavity 4038 sized and shaped to receive the sensor package. The cavity 4038 is disposed at a distal side of the head 4034 and opens in a distal direction. The head 4034 may also include a distal surface 4035 surrounding the cavity 4038. The cavity 4038 may include a planar inner surface 4048 and a conical inner surface 4039 extending circumferentially about planar inner surface 4048. The conical inner surface 4039 may taper inwardly in a proximal direction and may be configured to interface with a corresponding conical surface on the sensor package to assist with positioning the sensor package within the cavity 4038. In one example, the sensor package may be centered within the cavity 4038.

As also shown in FIG. 4AF, the ring 4130 may include a major lumen 4042 at a center thereof configured for passage of the delivery catheter shaft 4010. The ring 4130 may also include a plurality of minor lumens arranged about the major lumen 4042. In one example, the ring 4130 may include four minor lumens 4044 for control lines, such as lock lines or proximal element lines, arranged at 90-degree intervals about the major lumen 4042. The ring 4130 may also include four minor lumens 4046 for sensor leads arranged at 45-degree offsets from minor lumens 4044. Thus, the ring 4130 may include a total of eight minor lumens arranged about the major lumen 4042 at 45-degree intervals. However, more or fewer lumens are possible depending on sensor requirements and fixation device control requirements. The ring 4130 may be connected to the delivery catheter such that the minor lumens 4044, 4046 of the ring 4130 align with corresponding lumens in the delivery catheter.

Referring now to FIG. 4AD, which illustrates a standoff distance between the ring 4130 and the fixation device 500. A standoff distance D1 may be defined between free ends 4019 of the distal elements 702a, 702b and the distal surface 4035 of the head 4034 when the fixation device 500 is in a closed position such that the first and second clamps 4004a, 4004b are at their highest point relative to the ring 4130. Having a suitable standoff distance provides clearance such that the fixation device 500 may open and close freely without interference from a sensor package disposed in the ring 4130. The standoff distance D1 may vary depending on a clamp length L1 of the fixation device 500, with the clamp length L1 being defined along each of the first and second clamps 4004a, 4004b. In one example, the clamp length L1 may be about 9 mm, and the standoff distance D1 between the distal surface 4035 of the head 4034 and the free ends 4019 of the clamps 4004a, 4004b may be about 0.23 inches, or about 5.8 mm. In another example, the clamp length L1 may be about 12 mm, and the standoff distance D1 may be about 0.146 inches, or about 3.7 mm. In some embodiments, the standoff distance D1 may be within a range of plus or minus 5% of these values.

Referring now to FIG. 4AG-4AI, one or more sensor packages according to an embodiment of the present disclosure may be disposed within a ring 4130. The ring 4130 is similar to the ring 4130 described above with respect to FIG. 4AD-4AF. As shown in FIG. 4AH, the ring 4130 may include a body 4132 extending proximally from a head 4134, and a shaft 4136 extending proximally from the body 4132. In this embodiment, a first sensor package 4150a and a second sensor package 4150b may be disposed within the ring 4130 and may include respective first and second sensor-bearing surfaces 4152a, 4152b. The first and second sensor-bearing surfaces 4152a, 4152b may each be a flat, angled surface. In this regard, the first and second sensor-bearing surfaces 4152a, 4152b are not curved in either a first plane parallel to a central axis of the ring 4130 or a second plane perpendicular to the central axis of the ring 4130. Rather, the first and second sensor-bearing surfaces 4152a, 4152b may be angled relative to the central axis A of the ring 4130. Emitter-detector pairs 4170 may be disposed on each of the first and second sensor-bearing surfaces 4152a, 4152b and may be directed toward respective proximal elements 4140a, 4140b of a fixation device.

As shown in FIG. 4AG and 4AH, the first sensor package 4150a and the second sensor package 4150b may be disposed on opposite sides of a delivery catheter shaft 4110 within the ring 4130. The first sensor-bearing surface 4152a of the first sensor package 4150a and the second sensor-bearing surface 4152b of the second sensor package 4150b may each be angled such that the first and second sensor-bearing surfaces 4152a, 4152b each extend distally toward the delivery catheter shaft 4110. Together, the first and second sensor-bearing surfaces 4152a, 4152b may form a V-shape when viewed in front elevation. In the embodiment depicted, the planes of the first and second sensor-bearing surfaces 4152a, 4152b converge, but the first and second sensor-bearing surfaces 4152a, 4152b themselves do not converge. However, in other examples, the first and second sensor-bearing surfaces 4152a, 4152b themselves may converge. In other embodiments, a single, unitary sensor package may be provided having both the first sensor-bearing surface 4152a and the second sensor-bearing surface 4152b. The first and second sensor packages 4150a, 4150b may include openings 4162 for control lines, such as proximal element lines and lock lines. In the example depicted, one opening 4162 may extend through the first sensor package 4150a for passage of a proximal element line that controls a first proximal element 4140a, and another opening 4162 may extend through the second sensor package 4150b for passage of a proximal element line that controls a second proximal element 4140b.

As shown in FIG. 4AH, a central axis A may extend axially along the ring 4130 and the delivery catheter shaft 4110. An angle a may be measured from the central axis A to each of the first sensor-bearing surface 4152a and the second sensor-bearing surface 4152b. The angle a may be in a range of about 190 degrees to about 210 degrees from the central axis A, or about 15 degrees to about 25 degrees from a plane perpendicular to the central axis A, plus or minus 5 degrees. The angular relationship of the first and second sensor-bearing surfaces 4152a, 4152b may be symmetrical about the central axis A. The angle a may be selected such that the emitter-detector pairs 4170 disposed on the first and second sensor-bearing surfaces 4152a, 4152b are directed toward a gripping device of the fixation device 500 when first and second clamps of the fixation device 500 are in an open position. In one example, the angle a may be selected such that an optical axis of each emitter-detector pair 4170 is directed toward a location along a length of a corresponding proximal element that is at about 50% of a length of the corresponding proximal element 610a, 610b as measured from a free end thereof.

As shown in FIG. 4AG and 4AL, one or more emitter-detector pairs 4170 may be disposed on the first and second sensor-bearing surfaces 4152a, 4152b. In one example, four emitter-detector pairs 4170 may be provided, with two emitter-detector pairs 4170 disposed on the first sensor-bearing surface 4152a and two emitter-detector pairs 4170 disposed on the second sensor-bearing surface 4152b. However, more or fewer emitter-detector pairs 4170 may be provided depending on sensing requirements. For example, in some embodiments, one emitter-detector pair 4170 may be disposed on each of the first and second sensor-bearing surfaces 4152a, 4152b, for a total of two emitter-detector pairs 4170. In other embodiments, three or more emitter-detector pairs 4170 may be disposed on each of the first and second sensor-bearing surfaces 4152a, 4152b. Each emitter-detector pair 4170 may include an emitter E and a detector D disposed adjacent one another on the corresponding sensor-bearing surface. In the example of four emitter-detector pairs 4170, first and second emitter-detector pairs 4170a, 4170b may be disposed on the first sensor-bearing surface 4152a and may be directed toward a first proximal element 610a of a gripping device 600. Third and fourth emitter-detector pairs 4170c, 4170d may be disposed on the second sensor-bearing surface 4152b and may be directed toward a second proximal element 610b of the gripping device 600. The first and second emitter-detector pairs 4170a, 4170b may be laterally offset from a centerline of the first proximal element 610a such that an optical axis of each of the first and second emitter-detector pairs 4170a, 4170b is positioned adjacent a respective side edge 4142 of the first proximal element 610a. The third and fourth emitter-detector pairs 4170c, 4170d may be similarly laterally offset from a centerline of the second proximal element 610b such that an optical axis of each of the third and fourth emitter-detector pairs 4170c, 4170d is positioned adjacent a respective side edge 4142 of the second proximal element 610b. In this configuration, the first emitter-detector pair 4170a may be positioned adjacent a first side edge 4142 of the first proximal element 610a, and the second emitter-detector pair 4170b may be positioned adjacent a second side edge 4142 of the first proximal element 610a opposite the first side edge 4142. Additionally, the third emitter-detector pair 4170c may be positioned adjacent a first side edge 4142 of the second proximal element 610b, and the fourth emitter-detector pair 4170d may be positioned adjacent a second side edge 4142 of the second proximal element 610b opposite the first side edge 4142 of the second proximal element 610b. Because the side edges 4142 of the first and second proximal elements 610a, 610b define boundaries beyond which captured tissue may extend, positioning the emitter-detector pairs 4170a, 4170b, 4170c, 4170d adjacent the side edges 4142 may facilitate detection of tissue extending beyond the side edges 4142 of the first and second proximal elements 610a, 610b. Also, positioning the emitter-detector pairs 4170a, 4170b, 4170c, 4170d adjacent the side edges 4142 may reduce the likelihood that the first and second proximal elements 610a, 610b obscure the light emitted from the emitters E.

Referring now to FIG. 4AI, which depicts the relationship of the emitter-detector pairs 4170a, 4170b, 4170c, 4170d relative to the gripping device 600 having the first and second proximal elements 610a, 610b. A first plane P1 and a second plane P2 are orthogonal and each bisect the ring 4130. Second plane P2 extends along a length of the first and second proximal elements 610a, 610b and bisects their respective widths W1. First and second planes P1, P2 divide the emitter-detector pairs 4170a, 4170b, 4170c, 4170d into quadrants, with one emitter-detector pair 4170 disposed in each quadrant. The first and second proximal elements 610a, 610b each have a first width W1 defined between the side edges 4142 thereof. The first and second emitter-detector pairs 4170a, 4170b have a collective second width W2, and the third and fourth emitter-detector pairs 4170c, 4170d also have a collective second width W2. The widths W1 and W2 are measured parallel to first plane P1 and perpendicular to the second plane P2. The second width W2 is greater than the first width W1 such that the emitter-detector pairs 4170a, 4170b, 4170c, 4170d extend beyond the side edges 4142 of the first and second proximal elements 610a, 610b. This width relationship facilitates directing light toward and beyond the side edges 4142 of the first and second proximal elements 610a, 610b to define a width component of a detection zone, which may assist in assessing the adequacy of tissue capture by the gripping device 600.

In operation, distal elements of the fixation device 500 may be positioned at an open position to receive tissue between first and second clamps of the fixation device 500. When it is believed that tissue has been captured between first and second clamps by the first and second proximal elements 610a, 610b, the emitters E of the emitter-detector pairs 4170a, 4170b, 4170c, 4170d emit light toward regions adjacent the side edges 4142 of the first and second proximal elements 610a, 610b. Light emitted from the emitters E may intersect tissue captured within first and second clamps and may be at least partially reflected back to the detectors D of the emitter-detector pairs 4170a, 4170b, 4170c, 4170d. Reflected light received by the detectors D may be transmitted to the signal processing unit 4101 for generation of a signal indicative of one or more characteristics of the tissue, or signal generated by the detectors D may be transmitted to the signal processing unit 4101. In this regard, in implementations in which the emitters E and the detectors D are optical fibers, the reflected light may be directed through the detector optical fibers to the signal processing unit 4101. In implementations in which the emitters E and the detectors D are discrete emitter and detector elements mounted on the sensor-bearing surface, the detectors D may generate electrical signals indicative of the reflected light, and the electrical signals may be transmitted through electrical leads to the signal processing unit 4101. In either implementation, the signal generated based on the reflected light may be used to determine the presence or absence of tissue adjacent each side edge 4142 of the first and second proximal elements 610a, 610b, and thus to determine whether tissue is adequately captured by first and second clamps.

Because the first and second sensor-bearing surfaces 4152a, 4152b are each flat surfaces, the emitter-detector pairs 4170 disposed on a common sensor-bearing surface may emit light at substantially the same angle relative to the central axis A. The angle a of the first and second sensor-bearing surfaces 4152a, 4152b may be selected such that the emitter-detector pairs 4170 are directed toward a location along a length of the first and second proximal elements 610a, 610b that is at about 50% of a grip length of the first and second proximal elements 610a, 610b as measured from a free end thereof. Tissue insertion to at least 50% of the grip length may be associated with sufficient pinch force for secure tissue capture and reduced risk of single leaflet device attachment.

Accordingly, the angular relationship of the first and second sensor-bearing surfaces 4152a, 4152b may facilitate detection of tissue at a clinically significant insertion depth associated with secure capture and reduced risk of single leaflet device attachment. In some implementations, the emitter-detector pairs 4170 disposed on a common sensor-bearing surface may be offset from one another along a length direction of a corresponding proximal element 610a, 610b such that each emitter-detector pair 4170 interrogates a different location along the length of the corresponding proximal element 610a, 610b. Such axial offset between the emitter-detector pairs 4170 may permit assessment of tissue insertion depth along the length of the first and second proximal elements 610a, 610b at multiple locations in addition to the about 50% location.

Referring now to FIG. 4AJ-4AK, a sensor package 4250 according to a further embodiment of the present disclosure may be disposed within a ring 4230. The ring 4230 is similar to the ring 4130. As shown in FIG. 4AJ, the ring 4230 may include a body 4232 extending proximally from a head 4234, and a shaft 4236 extending proximally from the body 4232. In this embodiment, the sensor package 4250 may be disposed within the ring 4230 and may include a sensor-bearing surface 4258. Unlike the first and second sensor-bearing surfaces 4152a, 4152b of the embodiment of FIG. 4AG-4AI, which are flat surfaces, the sensor-bearing surface 4258 of the sensor package 4250 may be curved in both a first plane parallel to a central axis of the ring 4230 and a second plane perpendicular to the central axis of the ring 4230. For example, the sensor-bearing surface 4258 may be a dome surface or a semi-spherical surface. A plurality of emitter-detector pairs 4270 may be disposed on the sensor-bearing surface 4258 and may be directed toward a gripping device of a fixation device. The curvature of the sensor-bearing surface 4258 may cause the emitter-detector pairs 4270 disposed at different locations on the sensor-bearing surface 4258 to emit light at different angles and at different heights relative to the ring 4230, thereby permitting detection of tissue across multiple zones along a length of the gripping device.

Referring to FIG. 4AJ and 4AK, the sensor package 4250 may be disposed within the ring 4230 and may include one or more mating surfaces extending from a proximal side to a distal side thereof for engagement with the ring 4230. At a proximal side of the sensor package 4250, a planar surface 4252 may be provided for mating with a planar inner surface of the ring 4230. Extending distally from the planar surface 4252, a conical surface 4254 may be provided for mating with a conical inner surface of the ring 4230, which may assist in centering the sensor package 4250 within the head 4234 of the ring 4230. Additionally, a cylindrical surface 4256 may extend distally from the conical surface 4254. The sensor-bearing surface 4258 may extend distally from the cylindrical surface 4256 and may provide a surface on which the emitter-detector pairs 4270 are disposed. The sensor package 4250 may also include a central opening 4264 coaxial with a major lumen of the ring 4230 for passage of a delivery catheter shaft 4210 through the sensor package 4250. The sensor package 4250 may further include a plurality of peripheral openings 4262 configured to align with minor lumens of the ring 4230 for passage of control lines, such as proximal element lines and lock lines.

As best shown in FIG. 4AK, the sensor-bearing surface 4258 may have a radius of curvature in a range of about 0.05 inches to about 0.15 inches. In one example, the radius of curvature may be in a range of about 0.08 inches to about 0.12 inches. The curvature of the sensor-bearing surface 4258 in both the first plane parallel to the central axis of the ring 4230 and the second plane perpendicular to the central axis of the ring 4230 may cause the emitter-detector pairs 4270 disposed at different locations on the sensor-bearing surface 4258 to emit light at different angles toward the gripping device. The emitter-detector pairs 4270 disposed at a central region of the sensor-bearing surface 4258 closer to the central axis of the ring 4230 may emit light at angles directed more distally along the central axis, whereas the emitter-detector pairs 4270 disposed at peripheral regions of the sensor-bearing surface 4258 further from the central axis of the ring 4230 may emit light at angles directed more outwardly toward respective first and second proximal elements 610a, 610b. Additionally, the curvature of the sensor-bearing surface 4258 may cause the emitter-detector pairs 4270 to be disposed at different heights relative to the ring 4230, with the emitter-detector pairs 4270 disposed at a more proximal location on the sensor-bearing surface 4258 being at a different height than the emitter-detector pairs 4270 disposed at a more distal location on the sensor-bearing surface 4258. This height differential may further affect the angle at which light is directed toward the first and second proximal elements 610a, 610b. As a result, the emitter-detector pairs 4270 disposed on the sensor-bearing surface 4258 may collectively cover a range of emission angles allowing interrogation of tissue across multiple zones along a length of the gripping device between open and closed positions of the fixation device.

As shown in FIG. 4AJ, one or more emitter-detector pairs 4270 may be disposed on the sensor-bearing surface 4258 of the sensor package 4250. In one example, as shown in FIG. 4AL, eight emitter-detector pairs 4270a, 4270b, 4270c, 4270d, 4270e, 4270f, 4270g, 4270h may be provided on the sensor-bearing surface 4258. However, more or fewer emitter-detector pairs 4270 may be provided depending on sensing requirements. For example, in some embodiments, as few as two emitter-detector pairs 4270 may be provided on the sensor-bearing surface 4258. In other embodiments, four, six, or more than eight emitter-detector pairs 4270 may be provided on the sensor-bearing surface 4258. As best shown in FIG. 4AK and 4AL, each emitter-detector pair 4270 may include an emitter E and a detector D disposed adjacent one another on the sensor-bearing surface 4258.

In the example of eight emitter-detector pairs 4270, the emitter-detector pairs 4270 may be arranged in quadrants on the sensor-bearing surface 4258, with two emitter-detector pairs 4270 disposed in each quadrant. First and second emitter-detector pairs 4270a, 4270b may be disposed in a first quadrant and may be directed toward the first proximal element 610a of the gripping device 600. Third and fourth emitter-detector pairs 4270c, 4270d may be disposed in a second quadrant opposite the first quadrant and may also be directed toward the first proximal element 610a. Fifth and sixth emitter-detector pairs 4270e, 4270f may be disposed in a third quadrant and may be directed toward the second proximal element 610b of the gripping device 600. Seventh and eighth emitter-detector pairs 4270g, 4270h may be disposed in a fourth quadrant opposite the third quadrant and may also be directed toward the second proximal element 610b.

Within each quadrant, the two emitter-detector pairs 4270 disposed therein may be axially offset from one another along a length direction of the corresponding proximal element 610a, 610b. For example, one emitter-detector pair 4270 within a quadrant may be disposed at a more proximal axial location along the length direction, and the other emitter-detector pair 4270 within the same quadrant may be disposed at a more distal axial location along the length direction. Due to the curvature of the sensor-bearing surface 4258, the two emitter-detector pairs 4270 within a common quadrant may emit light at different angles and at different heights relative to the ring 4230, such that each emitter-detector pair 4270 within a common quadrant interrogates a different zone along the length of the corresponding proximal element 610a, 610b.

The first and second emitter-detector pairs 4270a, 4270b may be laterally offset from a centerline of the first proximal element 610a such that an optical axis of each of the first and second emitter-detector pairs 4270a, 4270b is positioned adjacent a first side edge 4242 of the first proximal element 610a. The third and fourth emitter-detector pairs 4270c, 4270d may be laterally offset from the centerline of the first proximal element 610a such that an optical axis of each of the third and fourth emitter-detector pairs 4270c, 4270d is positioned adjacent a second side edge 4242 of the first proximal element 610a opposite the first side edge 4242. The fifth and sixth emitter-detector pairs 4270e, 4270f may be laterally offset from a centerline of the second proximal element 610b such that an optical axis of each of the fifth and sixth emitter-detector pairs 4270e, 4270f is positioned adjacent a first side edge 4242 of the second proximal element 610b. The seventh and eighth emitter-detector pairs 4270g, 4270h may be laterally offset from the centerline of the second proximal element 610b such that an optical axis of each of the seventh and eighth emitter-detector pairs 4270g, 4270h is positioned adjacent a second side edge 4242 of the second proximal element 610b opposite the first side edge 4242 of the second proximal element 610b. Because the side edges 4242 of the first and second proximal elements 610a, 610b define boundaries beyond which captured tissue may extend, positioning the emitter-detector pairs 4270a, 4270b, 4270c, 4270d, 4270e, 4270f, 4270g, 4270h adjacent the side edges 4242 may facilitate detection of tissue extending beyond the side edges 4242 of the first and second proximal elements 610a, 610b.

Referring now to FIG. 4AL, which depicts the relationship of the emitter-detector pairs 4270a, 4270b, 4270c, 4270d, 4270e, 4270f, 4270g, 4270h relative to the gripping device 600 having the first and second proximal elements 610a, 610b. A first plane P1 and a second plane P2 are orthogonal and each bisect the ring 4230 and the sensor package 4250. Second plane P2 extends along a length of the first and second proximal elements 610a, 610b and bisects their respective widths W1. First and second planes P1, P2 divide the sensor-bearing surface 4258 into quadrants, with two emitter-detector pairs 4270 disposed in each quadrant. The first and second proximal elements 610a, 610b each have a first width W1 defined between the side edges 4242 thereof. The first, second, third, and fourth emitter-detector pairs 4270a, 4270b, 4270c, 4270d have a collective second width W2, and the fifth, sixth, seventh, and eighth emitter-detector pairs 4270e, 4270f, 4270g, 4270h also have a collective second width W2. The widths W1 and W2 are measured parallel to first plane P1 and perpendicular to the second plane P2. The second width W2 is greater than the first width W1 such that the emitter-detector pairs 4270a, 4270b, 4270c, 4270d, 4270e, 4270f, 4270g, 4270h extend beyond the side edges 4242 of the first and second proximal elements 610a, 610b. This width relationship facilitates directing light toward and beyond the side edges 4242 of the first and second proximal elements 610a, 610b to define a width component of a detection zone, which may assist in assessing the adequacy of tissue capture by the gripping device 600.

In operation, distal elements of the fixation device 500 may be positioned at an open position of about 702 degrees or less relative to each other to receive tissue between first and second clamps of the fixation device 500. When it is believed that tissue has been captured between first and second clamps by the first and second proximal elements 610a, 610b, the emitters E of the emitter-detector pairs 4270a, 4270b, 4270c, 4270d, 4270e, 4270f, 4270g, 4270h emit light toward regions adjacent the side edges 4242 of the first and second proximal elements 610a, 610b. Light emitted from the emitters E may intersect tissue captured within first and second clamps and may be at least partially reflected back to the detectors D of the emitter-detector pairs 4270a, 4270b, 4270c, 4270d, 4270e, 4270f, 4270g, 4270h. Reflected light received by the detectors D may be transmitted to the signal processing unit 4101 for generation of a signal indicative of one or more characteristics of the tissue, or a signal generated by the detectors D may be transmitted to the signal processing unit 4101. In implementations in which the emitters E and the detectors D are optical fibers, the reflected light may be directed through the detector optical fibers to the signal processing unit 4101. In implementations in which the emitters E and the detectors D are discrete emitter and detector elements mounted on the sensor-bearing surface 4258, the detectors D may generate electrical signals indicative of the reflected light, and the electrical signals may be transmitted through electrical leads to the signal processing unit 4101. In either implementation, the signal generated based on the reflected light may be used to determine the presence or absence of tissue adjacent each side edge 4242 of the first and second proximal elements 610a, 610b, and thus to determine whether tissue is adequately captured by first and second clamps.

Because the sensor-bearing surface 4258 is curved in both the first plane P1 parallel to the central axis of the ring 4230 and the second plane P2 perpendicular to the central axis of the ring 4230, the eight emitter-detector pairs 4270a, 4270b, 4270c, 4270d, 4270e, 4270f, 4270g, 4270h may each emit light at a distinct angle and from a distinct height relative to the ring 4230. As a result, the emitter-detector pairs 4270a, 4270b, 4270c, 4270d, 4270e, 4270f, 4270g, 4270h may collectively interrogate multiple zones along a length of each of the first and second proximal elements 610a, 610b. For example, the emitter-detector pairs 4270 disposed at a more proximal axial location within a quadrant may interrogate a first zone along the length of the corresponding proximal element 610a, 610b, and the emitter-detector pairs 4270 disposed at a more distal axial location within the same quadrant may interrogate a second zone along the length of the corresponding proximal element 610a, 610b. In some examples, the first zone may correspond to a location along the length of the corresponding proximal element 610a, 610b that is about 25% of a length of the corresponding proximal element 610a, 610b as measured from a free end thereof. In other examples, the first zone may correspond to a location along the length of the corresponding proximal element 610a, 610b that is about 50% of the length as measured from the free end thereof. In further examples, the second zone may correspond to a location along the length of the corresponding proximal element 610a, 610b that is about 75% of the grip length as measured from the free end thereof. As described above, tissue insertion to at least 50% of the grip length may be associated with sufficient pinch force for secure tissue capture and reduced risk of single leaflet device attachment. By interrogating multiple zones along the length of the first and second proximal elements 610a, 610b, the emitter-detector pairs 4270a, 4270b, 4270c, 4270d, 4270e, 4270f, 4270g, 4270h may collectively confirm that tissue has reached the 50% threshold and that tissue is adequately distributed along the length of the first and second proximal elements 610a, 610b.

In both the embodiment of the first and second sensor packages 4150a, 4150b of FIG. 4AG-4AI and the embodiment of the sensor package 4250 of FIG. 4AJ-4AL, one or more sensor packages may be disposed within a cavity of a ring at a distal end of a delivery catheter shaft, such as the cavity 4038 of the ring 4130 described above with respect to FIG. 4AD-4AF. The first and second sensor packages 4150a, 4150b and the sensor package 4250 may each be made from a ceramic material, an engineering polymer, or a flexible printed circuit board material, for example. The first and second sensor packages 4150a, 4150b and the sensor package 4250 may each be coupled to the ring via medical-grade adhesives, ultrasonic welding, or a press-fit connection. Flush mounting of the first and second sensor packages 4150a, 4150b and the sensor package 4250 within the ring may be advantageous so that the emitters E and the detectors D disposed on the first and second sensor packages 4150a, 4150b and the sensor package 4250 do not interfere with functions of the fixation device, such as opening and closing thereof.

In both the embodiment of the first and second sensor packages 4150a, 4150b of FIG. 4AG-4AI and the embodiment of the sensor package 4250 of FIG. 4AJ-4AL, the emitter-detector pair 4170, 4270 may include an emitter E and a detector D disposed adjacent one another on a corresponding sensor-bearing surface. The emitter E and the detector D within an emitter-detector pair 4170, 4270 may be laterally offset from a centerline of a corresponding proximal element such that an optical axis of the emitter-detector pair 4170, 4270 is positioned adjacent a side edge of the proximal element.

Within each emitter-detector pair 4170, 4270, the emitter E and the detector D may be arranged perpendicular to, parallel to, or at other orientations relative to a length direction of the corresponding proximal element. In some implementations, the emitter E and the detector D within an emitter-detector pair 4170, 4270 may be oriented at slightly different angles relative to one another, such as due to a curvature of the sensor-bearing surface on which the emitter-detector pair 4170, 4270 is disposed. The angular offset between the emitter E and the detector D within the emitter-detector pair 4170, 4270 may reduce specular reflection of light and may enhance detection of diffuse light reflected from tissue within a detection zone of the emitter-detector pair 4170, 4270.

In both the embodiment of the first and second sensor packages 4150a, 4150b of FIG. 4AG-4AI and the embodiment of the sensor package 4250 of FIG. 4AJ-4AL, optical cross-talk between adjacent emitter-detector pairs 4170, 4270 may occur when light emitted from an emitter E of one emitter-detector pair 4170, 4270 is received by a detector D of another emitter-detector pair 4170, 4270. Optical cross-talk may be mitigated by one or more approaches. For example, the emitters E of the emitter-detector pairs 4170, 4270 may be activated sequentially in time, with each detector D being gated to receive light only during a time window corresponding to activation of the emitter E of its paired emitter-detector pair 4170, 4270. Additionally or alternatively, optical isolation structures, such as opaque walls, baffles, or septa, may be disposed between adjacent emitter-detector pairs 4170, 4270 within the first and second sensor packages 4150a, 4150b and the sensor package 4250 to limit propagation of light between adjacent emitter-detector pairs 4170, 4270. Additionally or alternatively, the emitters E of the emitter-detector pairs 4170, 4270 may be configured to emit light with a limited angular spread to reduce light received by the detectors D of other emitter-detector pairs 4170, 4270.

In both the embodiment of the first and second sensor packages 4150a, 4150b of FIG. 4AG-4AI and the embodiment of the sensor package 4250 of FIG. 4AJ-4AL, the emitters E and the detectors D of the emitter-detector pairs 4170, 4270 are disposed on the ring 4130, 4230 at a distal end of a delivery catheter shaft 4110, 4210 and are spaced apart from a fixation device 3500 by a standoff distance, such as the standoff distance D1 described above. Accordingly, light emitted by the emitters E of the emitter-detector pairs 4170, 4270 toward the fixation device 3500 propagates through an anatomical space between the ring 4130, 4230 and the fixation device 3500 which may contain blood, prior to interacting with tissue captured by the fixation device 3500. Light reflected from the tissue similarly propagates through blood in the anatomical space prior to reception by the detectors D of the emitter-detector pairs 4170, 4270. The emitters E of the emitter-detector pairs 4170, 4270 may be configured to emit light at wavelengths selected to propagate through blood with reduced interference from absorption by water, hemoglobin, or both, as described above with respect to the optical sensing system 4100. For example, the emitters E of the emitter-detector pairs 4170, 4270 may emit light at wavelengths in the infrared range of about 500 nm to about 1,000 nm, or in the near-infrared range of about 700 nm to about 1,500 nm. Selection of wavelengths in these ranges may facilitate propagation of light between the emitters E of the emitter-detector pairs 4170, 4270, the tissue captured by the fixation device 500, and the detectors D of the emitter-detector pairs 4170, 4270 across the standoff distance between the ring 4130, 4230 and a fixation device 3500.

In both the embodiment of the first and second sensor packages 4150a, 4150b of FIG. 4AG-4AI and the embodiment of the sensor package 4250 of FIGS. 4AJ-4AL, the emitters E and the detectors D of the emitter-detector pairs 4170, 4270 may be coupled to sensor leads that extend from the first and second sensor packages 4150a, 4150b and the sensor package 4250 through the ring 4130, 4230 and through a delivery catheter, such as the delivery catheter 1230 described above, to the delivery device handle 1210 of the delivery catheter. Sensor leads may pass through minor lumens of the ring 4130, 4230, such as the minor lumens 4046 described above, and through corresponding lumens in the delivery catheter 1230 to the delivery device handle 1210. In implementations in which the emitters E and the detectors D of the emitter-detector pairs 4170, 4270 are optical fibers, the sensor leads may be optical fibers that extend from the first and second sensor packages 4150a, 4150b and the sensor package 4250 through the ring 4130, 4230 and the delivery catheter 1230 to the delivery device handle 1210, where the optical fibers may be optically coupled to one or more light sources and one or more photodetectors of the optical sensing system 4100. In implementations in which the emitters E and the detectors D of the emitter-detector pairs 4170, 4270 are discrete emitter and detector elements mounted on the first and second sensor-bearing surfaces 4152a, 4152b or the sensor-bearing surface 4258, the emitters E may include a light source such as a light-emitting diode or laser diode, and the detectors D may include a detection element such as a photodiode, phototransistor, charge-coupled device, complementary metal-oxide-semiconductor device, or photomultiplier tube, as described above with respect to the optical sensors 4110 of the optical sensing system 4100. In such implementations, the sensor leads may be electrical leads that extend from the first and second sensor packages 4150a, 4150b and the sensor package 4250 through the ring 4130, 4230 and the delivery catheter 1230 to the delivery device handle 1210, where the electrical leads may be electrically coupled to the signal processing unit 4101 of the optical sensing system 4100.

FIGS. 5, 6A-6D, and 8S depict the fixation device 500 according to another implementation of the present disclosure. The fixation device 500 may generally include the pair of distal elements 700, the pair of proximal elements 610, a coupling member 530, an actuator 520, and a stud 540. The distal elements 700 may include elongate arms 710 in which each arm 710 has a proximal end portion 712, which may be rotatably connected to the coupling member 530, and a free end 714, as best shown in FIG. 5. The free ends 714 may each have a rounded shape to minimize interference with and trauma to surrounding tissue structures according to some examples. In some examples, each free end 714 defines a curvature extending about two axes 720, 722. A first axis 720 may be a longitudinal axis of each respective arm 710. Additionally, the arms 710 may each include an engagement surface 718 that may also be curved about the first axis 720 and may extend at least partially along a length of the arm 710 to the free end 714. Thus, in some examples, the engagement surfaces 718 may each have a cupped or concave shape which may maximize contact area engagement with tissue and may assist in grasping and holding the valve leaflets LF. Such cupped or concave shape may further allow the arms 710 to nest around the shaft 900 of an interventional tool 502 while in the closed position to minimize the profile of the fixation device 500. Thus, the arms 710 may be at least partially cupped or curved inwardly about their longitudinal axes 720 which may form a concavity extending along the first axis 720 which may nest the proximal elements 610 when in a lowered position thereof. A second axis 722 about which each free end 714 may be curved may extend perpendicular to the first axis 720, as is also shown in FIG. 5. The curvature about the second axis 722 may be a reverse curvature located at the most distal portion of the free ends 714. In addition to the dual curvature, the free ends 714 may flare outwardly at their respective longitudinal edges. It is believed that both the reverse curvature and flare help create an atraumatic configuration that minimizes trauma to the tissue engaged therewith.

In the nonlimiting implementation depicted, a transverse width across the engagement surfaces 718 (which is in the direction of the second axis 722 and determines the width of tissue engaged) may be at least about 2 mm, 3-10 mm in some examples, and about 4-6 mm in some examples. In some implementations, a wider engagement may be desired wherein the engagement surfaces 718 are larger, for example about 2 cm, or multiple fixation devices 500 may be used adjacent to each other. The arms 710 may also have a length of about 6-12 mm (defined along the first axis 720), and the engagement surfaces 718 may be configured to engage a length of tissue of about 4-10 mm along the longitudinal axis 720 of the arms 710 according to some examples. Also, as shown in the illustrated example, each arm 710 may include a plurality of openings 716 to enhance grip and to promote tissue ingrowth following implantation.

In some examples, the actuator 520 includes two link members or legs 510.

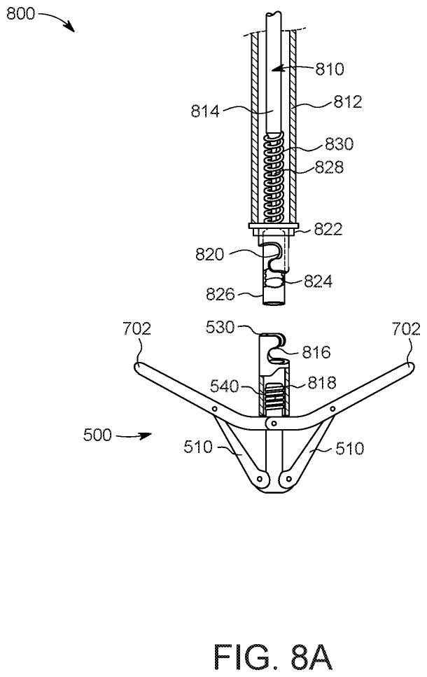

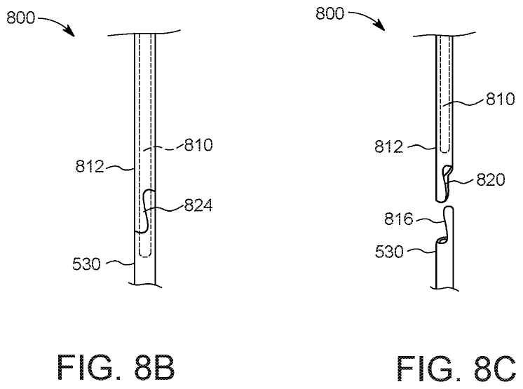

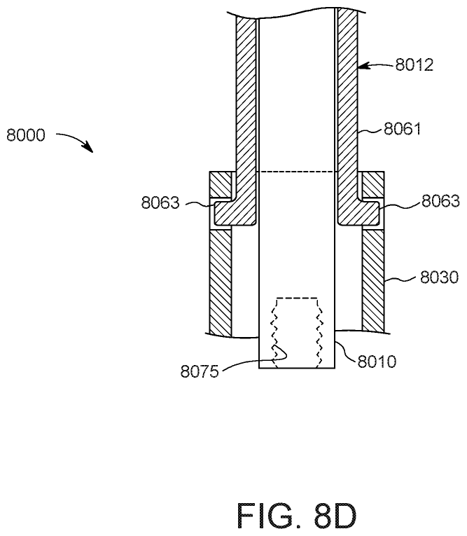

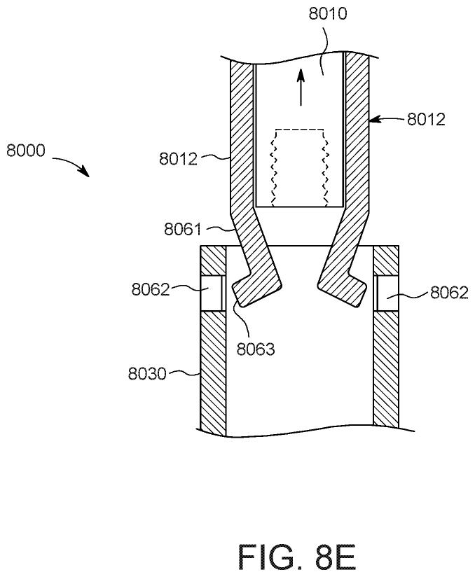

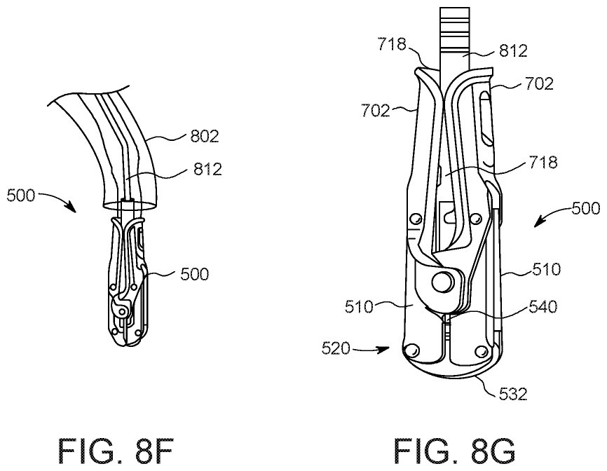

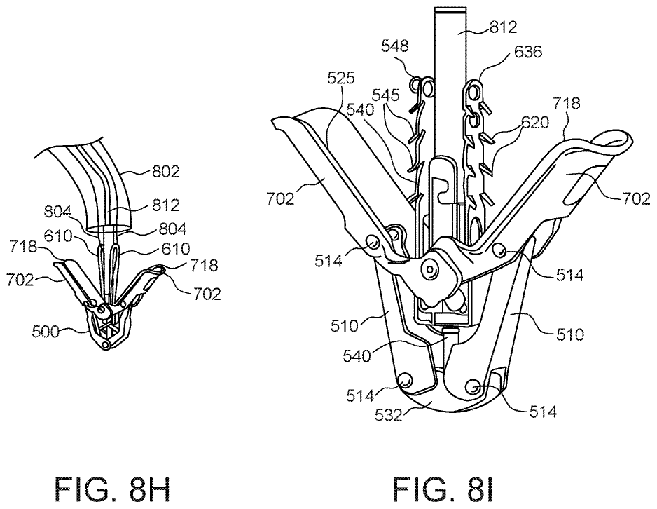

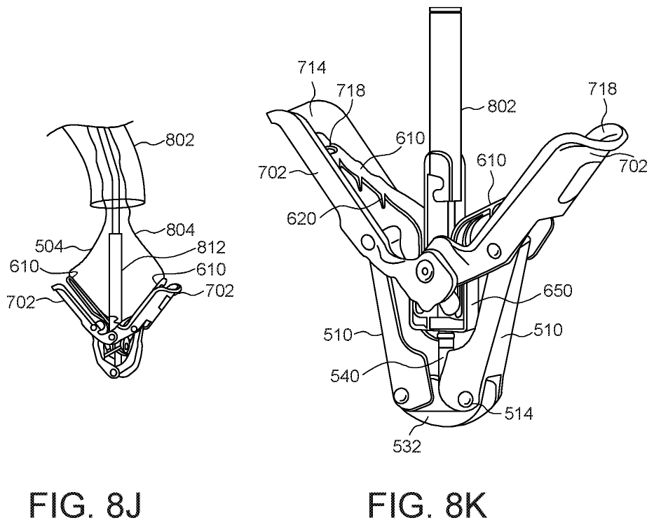

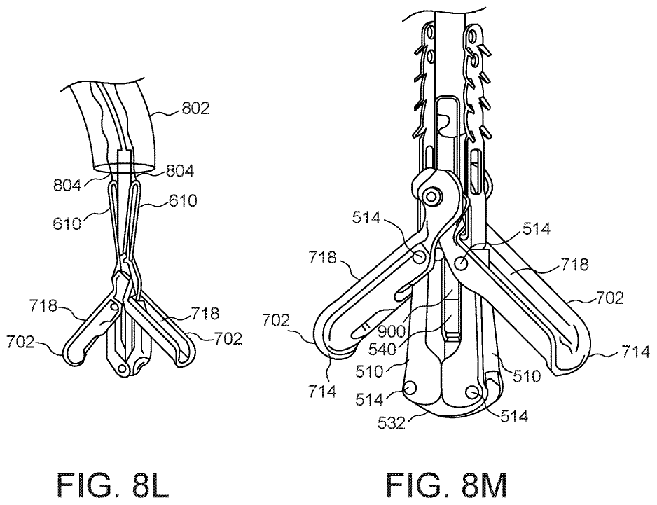

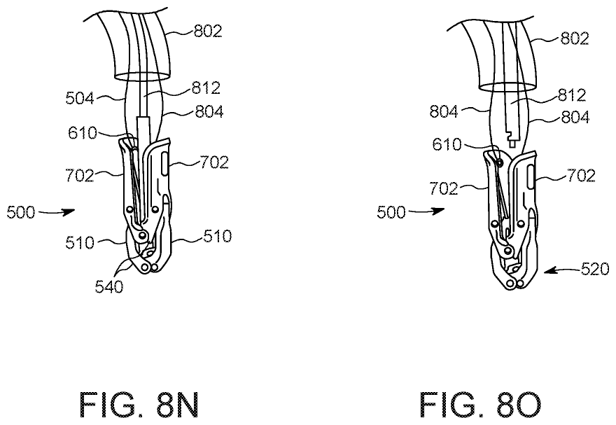

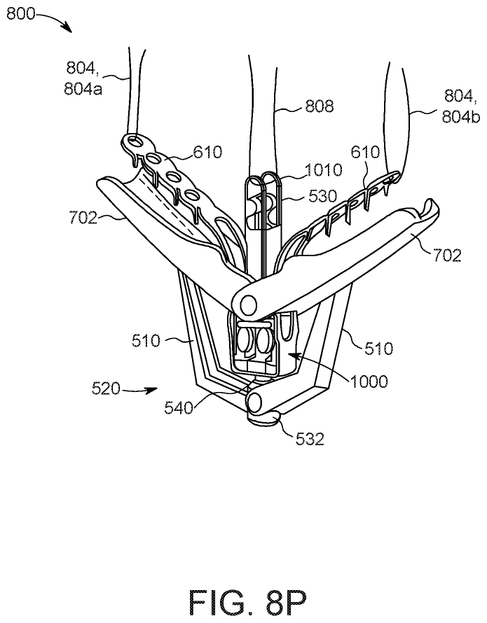

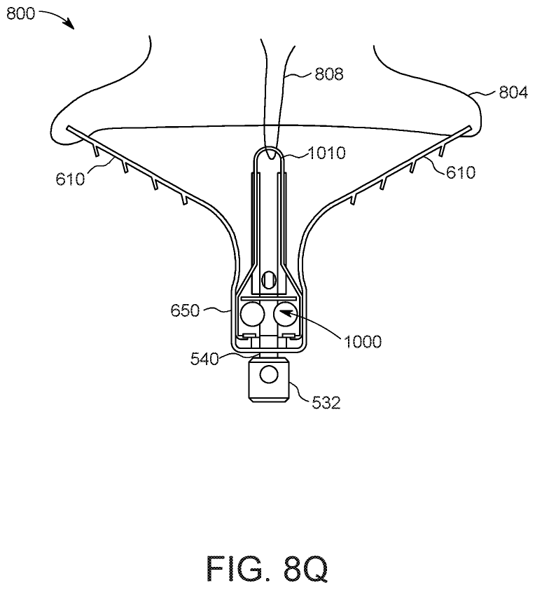

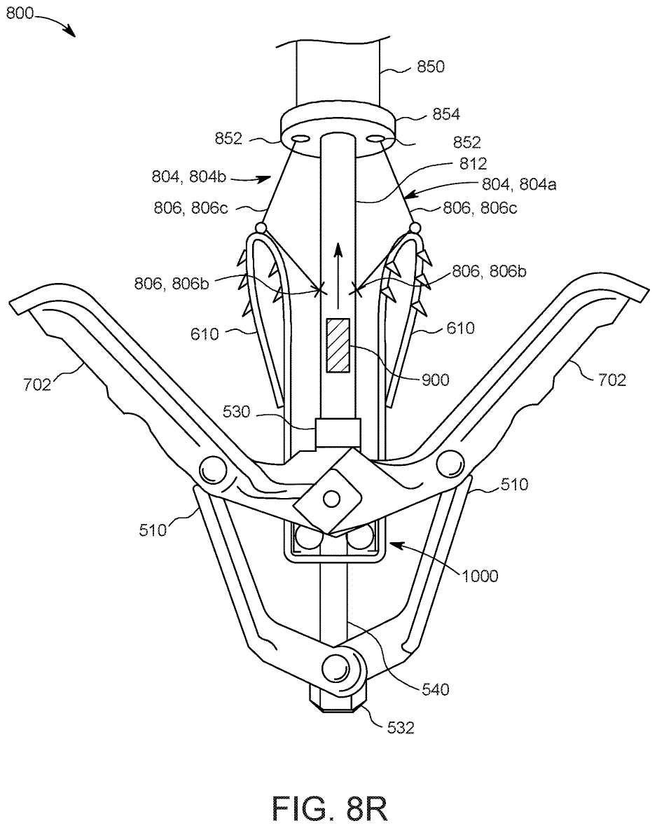

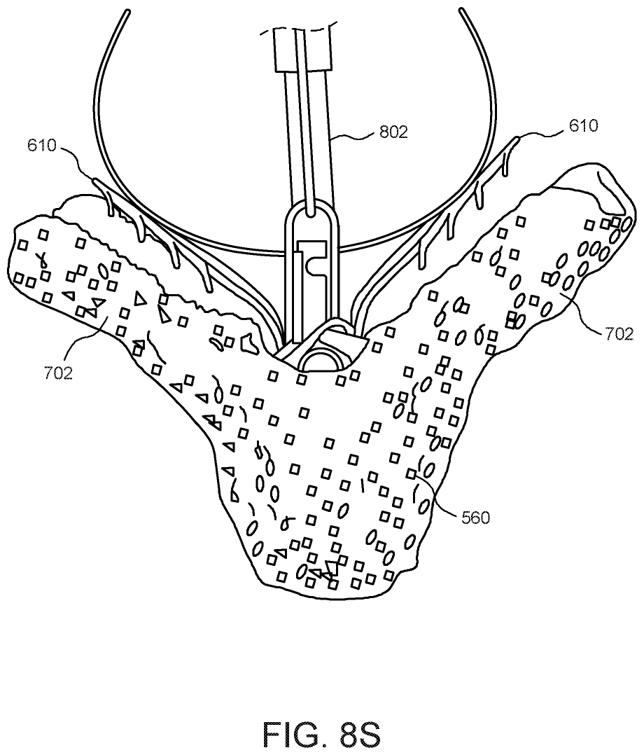

The legs 510 may be comprised of a rigid or semi-rigid metal or polymer such as Elgiloy®, cobalt chromium or stainless steel, however any suitable material may be used. Each leg 510 may have a first end 512, which may be rotatably joined with one of the distal elements 700 at a riveted joint 514, and a second end 516, which may be rotatably joined with the stud 540, as shown in FIG. 5. Although the depicted implementation shows both legs 510 pinned to the stud 540 by a single rivet 514, it is also contemplated that each leg 510 may be individually attached to the stud 540 by a separate rivet, pin or the like. In other implementations, the actuator 520 includes a base 822, and the second ends 516 of the legs 510 may be rotatably joined with the base 822, such as by one or more riveted joints 514, as best shown in FIG. 8K. An actuator rod 810 of the delivery system 1200 may be joinable with the actuator 520 directly, such as via direct connection with the base 822, or indirectly, such as via connection with the stud 540, which itself may extend from the base 822. In either of these implementations, the actuator rod 810 may be axially extendable and retractable in a proximal-distal direction to actuate the actuator 520 and consequently rotate the distal elements 700 between open, closed, and inverted positions, which are described further below. Additionally, the coupling member 530, the stud 540, and/or the base 822 may include a center portion or center body of the fixation device 500, for example.

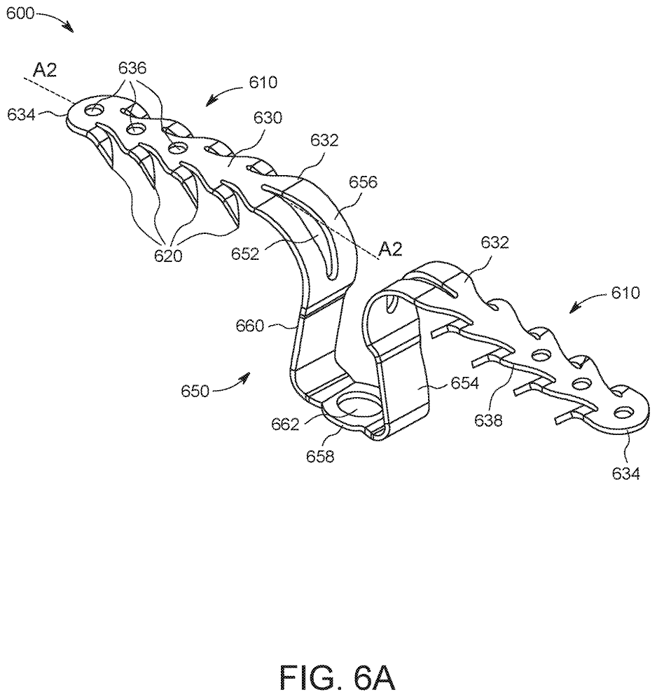

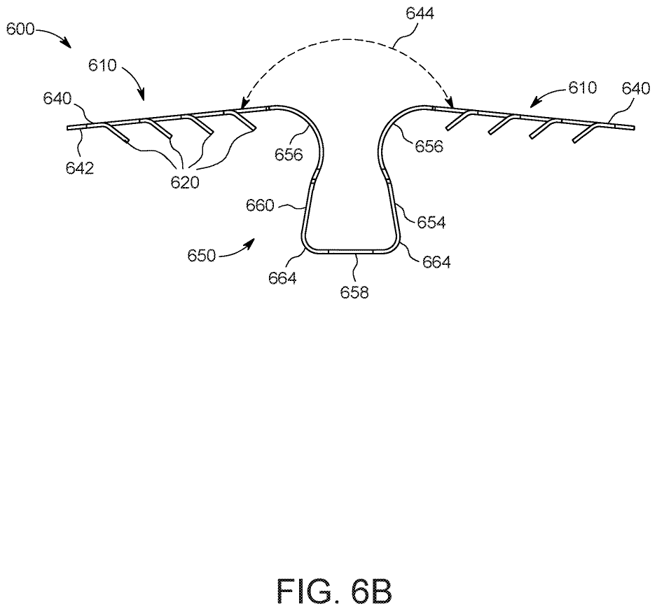

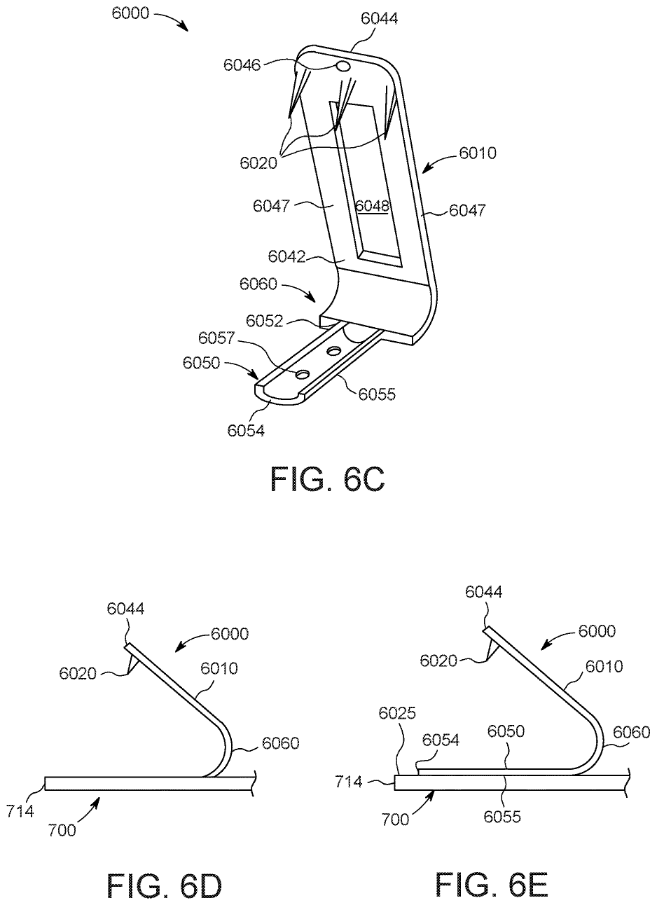

The proximal elements 610 may, in some examples, be flexible, resilient, and cantilevered from a center of the fixation device 500. For example, FIGS. 6A and 6B depict a gripping device (e.g., gripper arms) 600 that may generally include the pair of proximal elements 610, a base section 650, and a pair of arm bend features 656 partitioning the proximal elements 610 from the base section 650.

The proximal elements 610 may be in the form of elongate arms 630 that each extend along a longitudinal axis A2 from a first end portion or fixed end 632 to a second end portion or free end 634, as shown in FIG. 6A. Each proximal element 610 may also have opposed side edges 638 that define a width transverse to the longitudinal axis A2.

Such width may be less than the width of a corresponding distal element 700 such that the proximal element 610 may be recessed within the concavity formed by the engagement surface 718 of the distal element 700 when the proximal element 610 is moved into a lowered position, as described in more detail below.

The proximal elements 610 may also each have a first side or proximal side 640 and a second side or distal side 642. In some examples, the proximal elements 610 include a plurality of openings 636 that may extend from the proximal side 640 to the distal side 642, as shown in FIG. 6A. Such openings 636 may be used to couple a proximal element line, which is discussed further below, to the proximal element 610 for raising and lowering the proximal element 610. Each proximal element 610 may also include one or more frictional elements 620 extending from the distal side 642. For example, each proximal element 610 may include one or more rows of the frictional elements 620 where the frictional elements 620 in each row may be aligned in a direction transverse to the longitudinal axis A2. The frictional elements 620 in such rows may also be aligned with the frictional elements 620 in other rows in a lengthwise direction thereby forming columns of the frictional elements 620. For example, in the implementation depicted in FIGS. 6A and 6B, each proximal element 610 includes four rows of two frictional elements 620. In other words, two columns of four frictional elements 620. In other implementations, the proximal elements 610 include one to six rows of two to six frictional elements 620 per row, for example. However, in yet other implementations, the frictional elements 620 may be arranged in an offset relationship in a lengthwise and/or transverse direction such that at least some frictional elements 620 are not aligned with another frictional element 620 in such directions.

The frictional elements 620 may include frictional protrusions or tines having tapering pointed tips extending from the distal side 642 of the proximal elements 610.

Moreover, the frictional elements 620 may also be angled toward the fixed end 632 of the proximal element 610 which may help prevent the frictional elements 620 from inadvertently snaring tissue during repositioning of the fixation device 500. In some examples, the frictional elements 620 are integral with or connected to a distal surface 642 of the proximal element 620 and protrude therefrom. In additional examples, such as shown in FIG. 6A, the frictional elements 620 are formed from the side edges 638, such as by cutting and bending the base material forming the proximal elements 610, for example. Any suitable frictional elements 620 may be used, such as prongs, windings, bands, barbs, grooves, channels, bumps, surface roughening, sintering, high-friction pads, coverings, coatings, or a combination of these. However, some types of frictional elements 620 that can be utilized may permanently alter or cause some trauma to the tissue engaged. Thus, it may be advantageous for the frictional elements 620 to be atraumatic and generally frictional rather than penetrative so as to not injure or otherwise affect the tissue in a clinically significant way.

The base section 650 may be connected to a center portion of the fixation device 500 such that the proximal elements 610 extend outwardly therefrom. For example, the base section 650 may be coupled to the coupling member 530. In the implementation depicted, the base section 650 includes a first member 654, a second member 658, and a third member 660. The first member 654 and the third member 660 may be connected to the second member 658 to form a generally U-shaped or box-shaped structure which may allow a lock (discussed below) to be positioned between the first member 654 and the third member 660. However, other shapes may be formed, such as a V-shape, a crescent shape, or semicircular, for example. In some implementations, the first member 654 and the third member 660 are connected to the second member 658 via base bend features 664, for example. Also, the second member 658 may define an opening 662 extending therethrough for receipt of the stud 540 and/or the actuator rod 810, as shown in FIG. 6A.

The arm bend features 656 may couple the respective proximal element 610 and the base section 650. For example, an arm bend feature 656 can couple the proximal element 610 to the first member 654 of the base section 650, and another arm bend feature 656 can couple the other proximal element 610 to the third member 660 of the base section 650. As shown, the arm bend features 656 may form a living hinge about which the proximal elements 610 may bend relative to the base section 650. In this regard, the arm bend features 656 may be integral with the proximal elements 610 and the base section 650 and may bias the proximal elements 610 to a relaxed position. As illustrated in FIG. 6B, the proximal elements 610 may form a relaxed angle 644 formed between the proximal sides 640 of each proximal element 610. Such relaxed angle 644 is formed when the proximal elements 610 are in the relaxed position and may form an angle of about 85 degrees to 200 degrees (+/−5 degrees). For example, the proximal elements 610 may form a relaxed angle of 180 degrees in the relaxed position. In another example, the proximal elements 610 may form a relaxed angle of 185 degrees in the relaxed position. Although the implementation depicted illustrates the bend features 656 as living hinges, in other implementations the bend features 656 may include a biased hinge that modularly connects the proximal elements 610 to the base section 650. For example, the proximal elements 610 may be separately formed from the base section 650 and modularly connected to the base section 650 via the arm bend features 656 which may each include a spring biased hinge biasing the respective proximal element 610 to the relaxed position, for example.

The arm bend features 656 may also each include an elongate opening 652 extending along the longitudinal axis A2 which may furcate each arm bend feature 656, as illustrated in FIG. 6A. Such elongated opening 652 may have a uniform width extending along the longitudinal axis A2. However, in some implementations, such as the implementation depicted, the elongate opening 652 may form a bowling-pin shape such that a width of the opening 652 is narrower at one end (e.g., the end closest to the free end 634) than the other end (e.g., the end furthest from the free end 634) and is wider somewhere in between. The elongate opening 652 may also not be relegated to just the arm bend feature 656 but may also extend from the arm bend feature 656 to the proximal element 610 and/or the base section 650. The elongate opening 652 and corresponding furcation of the arm bend features 656 may be configured (e.g., in size, shape, spacing, position, etc.) so as to provide the desired resiliency, fatigue resistance, and/or flexibility at the coinciding arm bend features 656.

The base bend features 664 and the arm bend features 656 may be configured to give the gripping device 600 a bent configuration when the gripping device 600 is in a relaxed state (i.e., when the proximal elements 610 are in the relaxed position), such that when the gripping device 600 is forced into a stressed state (e.g., by bending the proximal elements 610 at one or more of the base bend features 664 and the arm bend features 656), the gripping device 600 is resiliently biased toward the relaxed state. In the exemplary implementation depicted, the gripping device 600 may be formed from a metallic sheet of a spring-like material, such as a shape-memory metal (e.g., Nitinol) which may provide the bias of the proximal elements 610 toward the relaxed position.

Alternatively, the gripping device 600 could be molded from a biocompatible polymer.

Each proximal element 610 may, in some examples, be configured to be at least partially recessed within the concavity of the distal element 700 when no tissue is present. When the fixation device 500 is in the open position, each proximal element 610 may be separated from the engagement surface 718 near the free end 714 of the arm 710 and may slope toward the engagement surface 718 near the free end 714 with the free end 634 of the proximal element 610 contacting the engagement surface 718, as illustrated in FIGS. 5 and 8K. This arrangement may be facilitated by the dimensions of the base section 650. For example, increasing or decreasing the respective lengths of the first member 654, the second member 658, and the third member 660 of the base section 650 may increase or decrease the separation distance between the proximal element 610 and the corresponding distal element 700 which may help accommodate a valve leaflet or other tissues of varying thicknesses. Further examples of gripping devices that may be utilized in the fixation device 500 are described in more detail in U.S. Pat. No. 11,096,691, the disclosure of which is incorporated by reference herein in its entirety.

In other implementations the proximal elements 610 may be connected to or otherwise extend from the distal elements 700 rather than from a center of the fixation device 500, like that of the fixation device 500. For example, FIGS. 6C and 6D depict a gripping device 6000 according to another implementation of the present disclosure that may generally include a first arm 6010, a second arm 6050, and an arm bend feature 8030 partitioning the first arm 6010 from the second arm 6050. The gripping device 6000 may be made from a memory-metal material, such as Nitinol, for example.

The first arm 6010 may constitute a proximal element of the fixation device 500, like that of and as an alternative to the proximal element 610 and may include one or more frictional elements 6020 which may be similar to the frictional elements 620 discussed above. Thus, a plurality of frictional elements 6020 may extend from a distal side of the first arm 6010 such as in one or more rows and/or columns. In the implementation depicted, a single row of three frictional elements 6020 may be provided near a free end 6044 of the first arm 6010. But, as mentioned above, the first arm 6010 may have any number of frictional elements 6020, such as two, four, or six, for example. The first arm 6010 may also include a pair of elongate members 6047 offset from each other to form a space 6048 therebetween. Such space 6048 may be configured to receive the second arm 6050, for example. Additionally, the first arm 6010 may include one or more openings 6046, such as near the free end 6044, as shown in FIG. 6C. Such opening 6046 may be configured to receive a proximal element line for raising and lowering the first arm 6010.

The second arm 6050 may be in the form of a beam or other elongate structure. The second arm 6050 (also referred to herein as a base section 6050) may be configured to couple to the distal element 700. For example, in the implementation depicted in FIGS. 6C and 6D, the second arm 6050 may be curved in a plane transverse to its longitudinal axis. For example, the second arm 6050 may be semi-cylindrical such that it may have a semi-circular profile. Thus, the second arm 6050 may have a convex surface 6055 configured to conform to the cupped curvature of the engagement surface 718 of the corresponding distal element 700. FIG. 6D illustrates the second arm 6050 coupled to the proximal engagement surface 718 of the distal element 700 such that it is generally recessed within the distal element 700 and the free ends 6044, 6054 of the first and second arms 6010, 6050 point in the general direction toward the free end 714 of the distal element 700. Thus, in some implementations, the second arm 6050 may have a width configured to be positioned within the concavity of the distal element 700 and secure to the proximal engagement surface 718. In other implementations, a second arm 6050 of an alternative gripping device 600 may not be concave and may instead have a planar surface corresponding to a planar engagement surface 718 of an alternative distal element 700′ and secured thereto, as illustrated in FIG. 6E. In further implementations, the distal element 700 may include a recess or pocket for receipt and securement of a second arm 250, such as in a press-fit manner, for example. The second arm 6050 may be secured to the distal element 700 in any number of ways, such as via one or more sutures, welding, press-fit, fastener (e.g., rivet or screw) or the like. For example, a rivet, screw, or suture may pass through one or more openings 6057 in the second arm 6050 and into the distal element 700. A tissue fixation device, such as the fixation device 500, may include a pair of gripping devices 6000 with one coupled to each distal element 700 as mentioned above.

The arm bend feature 8030 may be coupled to a fixed end 6042 of the first arm 6010 and a fixed end 6052 of the second arm 6050 such that the first arm 6010 and the second arm 6050 extend in the same general direction and may form a V-shape when the first arm 6010 is in an exemplary open or raised position, as illustrated in FIGS. 6D and 6E. As shown, the arm bend feature 6060 may form a living hinge about which the first arm 6010 may bend relative to the second arm 6050. In this regard, the arm bend feature 6060 may be integral with the first arm 6010 and the second arm 6050 so as to form a monolithic structure and may bias the first arm 6010 to a relaxed position. Such relaxed position may include the second arm 6050 extending through the space 6048 between the elongate members 6047 of the first arm 6010 to form an X-shape. However, it should be noted that such position can generally only be achieved when the gripping device 6000 is not coupled to the distal element 700 as the presence of the distal element 700 would prevent the second arm 6050 from passing into the space 6048. In some implementations of the gripping device 6000, the arm bend feature 6060 may be a spring loaded or otherwise biased hinge coupling separately formed first and second arms 6010, 6050.

The fixation device 500 may also have the covering 560, as shown in FIG. 8S. As depicted, the covering 560 may encapsulate the distal elements 700 and the actuator 520. Thus, the engagement surfaces 718 may be covered by the covering 560 which may help minimize trauma on tissues and enhance primary fixation via additional friction to assist in grasping. Additionally, the covering 560 on the engagement surfaces 718 may facilitate tissue ingrowth to provide for secondary fixation to ensure long-term security.

The covering 560 may be loosely fitted and/or may be flexible such that the fixation device 500 can freely move to various positions all the while the covering 560 conforms to the contours of the fixation device 500 and remains securely attached thereto. The covering 560 may cover specific parts of the fixation device 500 while leaving other parts exposed. For example, the proximal elements 610 may be exposed, while the distal elements 700 and the actuator 520 may be covered. However, in some implementations, the proximal elements 610 may be covered with the covering 560 to enhance grip and tissue ingrowth following implantation. Preferably, when the covering 560 is used in combination with the frictional elements 620 or other frictional features, such as those extending from the proximal elements 610, such features may protrude through such covering 560 so as to contact any tissue engaged by the proximal elements 610.

The covering 560 may be comprised of any biocompatible material, such as polyethylene terephthalate, polyester, cotton, polyurethane, expanded polytetrafluoroethylene (ePTFE), silicon, or various polymers or fibers and have any suitable form, such as a fabric (woven or unwoven), mesh, textured weave, felt, looped or porous structure. Generally, the covering 560 has a low profile so as not to interfere with delivery through the guide catheter 802 or with grasping and coapting of the leaflets LF or tissue. The covering 560 may alternatively be comprised of a polymer or other suitable materials dipped, sprayed, coated, or otherwise adhered to the surfaces of the fixation device 500. Optionally, a polymer coating may include pores or contours to assist in grasping the tissue and/or to promote tissue ingrowth. Any of the coverings 560 may optionally include drugs, antibiotics, anti-thrombosis agents, or anti-platelet agents such as heparin, COUMADIN® (Warfarin Sodium), to name a few. These agents may, for example, be impregnated in or coated on the coverings 560. These agents may then be delivered to the grasped tissues surrounding tissues and/or bloodstream for therapeutic effects.

FIGS. 8A-8C depict an exemplary coupling system 800 between the fixation device 500 and the shaft 900 of the delivery system 1200. As mentioned above, once the leaflets LF of a target valve are coapted in the desired arrangement, the fixation device 500 may then be detached from the delivery system 1200 and left behind as an implant to hold the leaflets LF together in the coapted position. Such detachment may occur between the coupling member 530 of the fixation device 500 and a distal end of the shaft 900. Thus, the coupling member 530 may be configured to be releasably coupled to the shaft 900. The coupling member 530 may be disposed at a center of the fixation device 500 and may extend proximally along its the longitudinal axis of the fixation device 500. In the coupling system 80 depicted, the shaft 900 may form a tubular upper shaft with a first mating surface 820 formed at a distal end thereof, and the coupling member 530 may form a detachable lower tubular shaft with a second mating surface 816 formed at a proximal end thereof. The mating surfaces 816, 820 may be correspondingly shaped so that they interlock and form a joining line 565 when merged together, as shown in FIG. 8B. In this regard, the mating surfaces 816, 820 may have any shape or curvature which allows or facilitates interlocking and later detachment. For example, in the depicted implementation, the mating surfaces 816, 820 define the joining line 565 with an S-shaped curvature.

The coupling system 800 may also include the actuator rod 810 and the stud 540 (or alternatively the base 532) such that the fixation device 500 may also be releasably coupled to the delivery system 1200 via connection between the actuator rod 810 and the stud 540. When the shaft 900 is coupled to the coupling member 530, they may collectively form an axial channel. The actuator rod 810 may pass through this channel to bridge the joining line 565, as shown in FIG. 8B. The actuator rod 810 may include a proximal extremity 814, a distal extremity 828, and a joiner 826. The distal extremity 828 may be smaller in diameter than the proximal extremity 814 and may be optionally surrounded by a coil 830 which may serve to bias the joiner 826 in a proximal direction. However, in some implementations, the actuator rod 810 may not have the coil 830 or the proximal and distal extremities 561, 828 of differing diameters. The joiner 826 may be removably coupled with the stud 540 of the fixation device 500 via any one of various possible release mechanisms. For example, in the implementation depicted, the joiner 826 may be threadedly connected to the stud 540 of the fixation device 500. In this regard, the joiner 826 may have internal threads 824 which mate with external threads 818 on the stud 540. Alternatively, the joiner 826 may have external threads which mate with internal threads of the stud 540. As described previously, the stud 540 may be connected with the distal elements 700 so that advancement and retraction of the stud 540, by means of the actuator rod 810, manipulates the distal elements 700. It is also contemplated that the joiner 826 may be directly threadedly engaged with the base 532 where no stud 540 is provided. Once detachment of the fixation device 500 is desired, the actuator rod 810 may be rotated until the threads 824 of the joiner 826 disengage the external threads 818 of the stud 540. The actuator rod 810 may then be retracted to a position above the mating surfaces 816, 820 which in turn allows the coupling member 530 to separate from the shaft 900 along the joining line 565, as illustrated in FIG. 8C.

FIGS. 8D and 8E illustrate an alternative example of a coupling system. In this exemplary coupling system 5015, a shaft 8012 of the delivery system 1200 may be releasably coupled with a coupling member 8030 via a detent mechanism, for example. In this regard, the shaft 8012 may form an upper tubular shaft with detent mechanism features and the coupling member 8030 may form a lower tubular shaft with detent mechanism features configured to releasably connect with the detent mechanism features of the shaft 8012. In the implementation depicted, the detent mechanism may include one or more spring arms 8061 integrally formed on the shaft 8012 and one or more receptacles 8062 sized to receive the spring arms 8061 within the coupling member 8030. However, the shaft 8012 may include the receptacles 8062, while the coupling member 8030 may include the spring arms 8061, for example. As shown, the spring arms 8061 may have a flange-like engagement element 8063 at a distal end thereof and are preferably biased inwardly, i.e., toward an interior shaft 8012, as shown in FIG. 8E. The receptacles 8062 may be configured to receive and mate with respective engagement elements 8063 of the spring arms 8061, as shown in FIG. 8D. The receptacles 8062 may extend all the way through the wall of the coupling member 8030 and may be sized to snuggly fit both engagement elements 8063. A snuggly fitting rod (such as an actuator rod 8010) may extend through the shaft 8012 and the coupling member 8030 and may outwardly deflecting the inwardly biased spring arms 8061 such that the engagement elements 8063 are pushed into respective engagement with a corresponding receptacle 8062 thereby coupling the shaft 8012 to the coupling member 8030, as shown in the example of FIG. 8D. When desirable to detach the fixation device 500 from the delivery system 1200, the actuator rod 8010 may be retracted to a position above the spring arms 8061 and the engagement features 8063 thereof. This allows the inwardly biased spring arms 8061 and corresponding engagement elements 8063 to disengage from the receptacles 8062 thereby detaching the shaft 8012 and the coupling member 8030. As mentioned above, the actuator rod 8010 may be threadedly engaged to the stud 540.

Thus, the actuator rod 8010 may first be rotated to unthread its threads 8075 from the stud 540 and then retracted to release the coupling member 8030 according to an example of the disclosure.

As mentioned above, the fixation device 500 may, in some examples, be actuated through multiple positions within a mammalian body during a transcatheter procedure such as by extending and retracting the actuator rod 810 when coupled to the stud 540 and/or the base 532. FIGS. 8F-80 illustrate several of these possible positions and in a sequence that may be utilized during a transcatheter procedure.

FIGS. 8F and 8G depict the fixation device 500 in an example of a closed position or delivery position. The fixation device 500 may assume the closed position when being delivered through the guide catheter 802 of the steerable guide system 1105, as shown in FIG. 8F. In the closed position, the opposed pair of distal elements 700 may be positioned so that the engagement surfaces 718 thereof face each other. The cupped or concave shape of each arm 710 in this example allows the arms 710 to surround the shaft 900 and optionally contact each other on opposite sides of the shaft 900. This provides a low profile for the fixation device 500 so that it is readily passable through a catheter 3330 and through any anatomical structures, such as those within the cardiovascular system.

FIGS. 8H and 8I depict the fixation device 500 in an example of an open position. The fixation device 500 may assume the open position for capturing and grasping the leaflets LF of a heart valve. In an open position, the distal elements 700 may be rotated so that the engagement surfaces 718 thereof face a first direction such that the engagement surfaces 718 are disposed at an acute angle relative to the shaft 900. For example, the acute angle formed between each engagement surface 718 and the shaft 900 may be 45 degrees to 90 degrees. Stated differently, in the open position, the engagement surfaces 718 of the distal elements 700 may be oriented 90 degrees to 180 degrees relative to each other. However, it is generally preferable for the arms 710 to be positioned 120 degrees relative to each other (and 60 degrees relative to the shaft 900) for capturing the leaflets LF. Movement of the fixation device 500 from the closed position to the open position may be achieved by advancing the stud 540 distally relative to the coupling member 530 by distally advancing the actuator rod 810. Conversely, the fixation device 500 may be moved from the open position to the closed position by retracting the actuator rod 810 and retracting the stud 540 proximally, according to some examples of the disclosure, As shown in FIG. 81, the proximal elements 610 (or the first arm 6010) may be in a raised or insertion position when the fixation device 500 is in the open position to facilitate insertion of the leaflets LF between the distal elements 700 and the proximal elements 610 for their capture. The proximal elements 610 are, in some examples, biased toward the distal elements 700. In this regard, the proximal elements 610 may be moved inwardly toward the shaft 900 and held against the shaft 900 with the aid of proximal element lines 804 which can be in the form of sutures, wires, nitinol wire, rods, cables, polymeric lines, or other suitable structures, as shown in FIG. 8H. Thus, FIGS. 8H and 8I depict the fixation device 500 in an insertion configuration in which the proximal elements 610 are in a raised position and the distal elements 700 are in an open position.

Once the fixation device 500 has been positioned in a desired location against the valve leaflets LF, the leaflets LF may then be captured between the proximal elements 610 and the distal elements 700. FIGS. 8J and 8K illustrate the fixation device 500 in an example of such a position. Here, the proximal elements 610 are lowered toward the engagement surfaces 718 so that the proximal elements 610 are in a lowered or capture position, and the leaflets LF are held between the distal elements 700 and the proximal elements 610. In the closed position, the engagement surface 5042 of the proximal fixation element 610 is in a position proximal to the engagement surface 5022 of the distal fixation element 700. The proximal elements 610 are, in some examples, lowered into the lowered position while the distal elements 700 remain in the open position. Thus, the fixation device 500, as shown in FIGS. 8J and 8K is in an example of a capture configuration which may be similar to the insertion configuration of FIGS. 8H and 8I, but with the difference being that the proximal elements 610 are now lowered toward the distal elements 700 by releasing tension on the proximal element lines 804 to compress the leaflet tissue therebetween. At any time, the proximal elements 610 may be raised and the distal elements 700 adjusted or inverted to reposition the fixation device 500 if regurgitation is not sufficiently reduced according to some examples of the disclosure.

FIGS. 8L and 8M depict an example of an inverted position of the fixation device 500. The fixation device 500 may assume the inverted position to aid in repositioning or removal of the fixation device 500. In some examples of the inverted position, the distal elements 700 may be further rotated from the open position, which may be achieved by advancing the stud 540 further relative to the open position, so that the engagement surfaces 718 of the distal elements 700 face outwardly, and the free ends 714 point distally. Additionally, in some examples, the engagement surfaces 718 of each arm 710 may form an obtuse angle relative to the shaft 900. For example, the obtuse angle formed between each engagement surface 718 and the shaft 900 may be 60 degrees to 180 degrees.

Also, as shown in FIG. 8M, in some examples the proximal elements 610 are in their raised position against the shaft 900 while the distal elements 700 are in the inverted position by exerting tension on the proximal element lines 501. Thus, a relatively large space may be created between the proximal elements 610 and the distal elements 700, for repositioning. In addition, the inverted position allows withdrawal of the fixation device 500 through the valve while minimizing trauma to the leaflets LF. The engagement surfaces 718 provide an atraumatic surface for deflecting tissue as the fixation device 500 is retracted proximally. It should be further noted that the frictional elements 620 of the proximal elements 610 may, in some examples, be angled slightly in the distal direction (away from the free ends 634 of the proximal elements 610), reducing the risk that the frictional elements 620 will catch on or lacerate tissue as the fixation device 500 is withdrawn and while the proximal elements 610 are in the raised position.

After the leaflets LF have been captured between the distal elements 700 and the proximal elements 610, the distal elements 700 may be returned to or toward the closed position where they may be locked in place. An example of such locking is described further below. FIG. 8N illustrates the fixation device 500 in the closed position wherein the leaflets LF (not shown) are captured and coapted. In some examples, this is achieved by retraction of the stud 540 proximally relative to the coupling member 530 so that the legs 510 of the actuator 520 apply an upwards force to the distal elements 700 which in turn rotate the distal elements 700 so that the engagement surfaces 718 again face one another, similar to that of FIGS. 8F and 8G, and so that the distal elements 700 rotate the proximal elements 610 in a direction toward the shaft 900. However, because the leaflets LF are captured between the distal elements 700 and the proximal elements 610, it may be desirable to keep the distal elements 700 at about 20 degrees to 60 degrees relative to each other so as to limit the amount of tension and stress on the native tissue. Thus, while the fixation device 500 may be returned to the closed position, such closed position may not be as closed as in the initial delivery position.

As shown in FIG. 80, the fixation device 500 may then be released from the shaft 900 of the delivery system 1200 while in the closed position. As mentioned, the fixation device 500 may be releasably coupled to the delivery system 1200 via a coupling system (e.g., the coupling system 800 or 5015). When the coupling structures of such coupling system are released, the proximal element lines 804 may remain attached to the proximal elements 610 following detachment to function as a tether to keep the fixation device 500 connected with the delivery catheter 850 (see FIG. 12B) for reconnection and repositioning. However, in other implementations, the proximal element lines 804 may be released prior to release of the fixation device 500 or concurrently with the release of the fixation device 500, as described in more detail below.

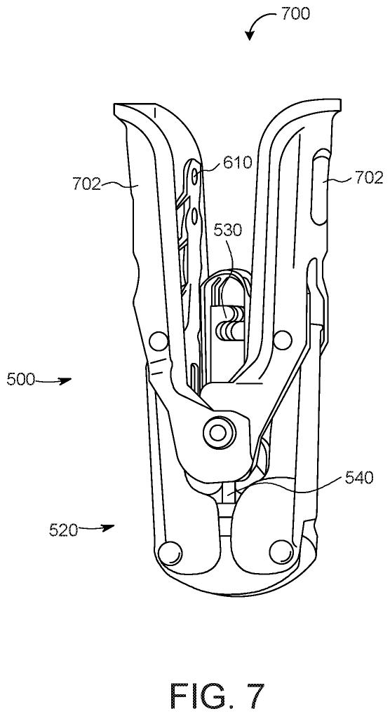

FIG. 7 illustrates a receiving device 700 of a released fixation device 500 in an example of a closed position. The receiving device 700 includes distal elements 702.

As shown, the coupling member 530 remains separated from the shaft 900 of the delivery system 1200, and the proximal elements 610 are deployed so that tissue (not shown) may reside between the proximal elements 610 and the distal elements 700.

As mentioned above, the proximal element lines 804 may be releasably coupled to the proximal elements 610. In some examples, the proximal element lines 804 may pass through an opening in the proximal elements 610, such as the openings 636 and 6046 in the case of the proximal element 610. In other examples, eyelets, which may be formed from one or more lengths of suture, may be coupled to the proximal elements 610 and the proximal element lines 804 may pass through such eyelets. Thus, the proximal element lines 804 may be released from the proximal elements 610 prior to, concurrent with, or after release of the fixation device 500 from the delivery system 1200 according to various examples.

In an exemplary implementation of the interventional tool 502, as shown in FIG. 8P, a plurality of proximal element lines 804a, 804b may be coupled to the proximal elements 610 of the fixation device 500. Each of a first proximal element line 804a and a second proximal element line 804b may be elongated flexible threads, wire, cable, sutures, or lines extending through the shaft 900, looped through the proximal elements 610, and extending back through the shaft 900 to the delivery device handle 1210 (see FIG. 12A) of the delivery system 1200. When detachment is desired, one end of each of the proximal element lines 804a, 804b may be released from the delivery system 1200, and the other end pulled to draw the free end distally through the shaft 900 and through the proximal element 610 thereby releasing it. Also, in this arrangement, the proximal element lines 804a and 804b may be independently or concurrently manipulated so as to independently or concurrently raise and lower the proximal elements 610, respectively.

In another example, the interventional tool 502 may be configured, as shown in FIG. 8Q with respect to certain components thereof, such that the proximal elements 610 may alternatively be supported by the single proximal element line 804 which may extend through both of the proximal elements 610. In this arrangement both proximal elements 610 may be raised and lowered concurrently by action of the single proximal element line 501. Whether the proximal elements 610 are manipulated individually by separate proximal element lines 804 or jointly by the single proximal element line 501, the proximal element lines 804 may extend directly through openings (e.g., the openings 618) of the proximal elements 610 and/or through a layer or portion of the covering 560 on the proximal elements 610, or through a suture loop/eyelet above or below the covering 560, for example.

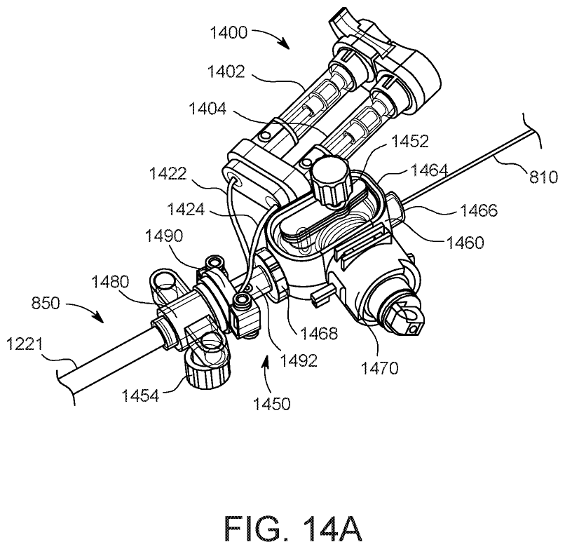

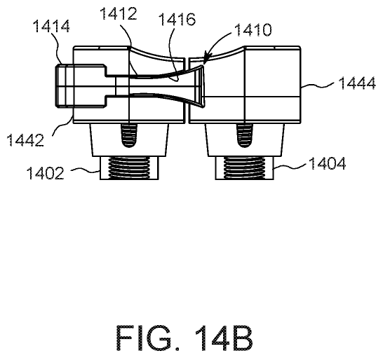

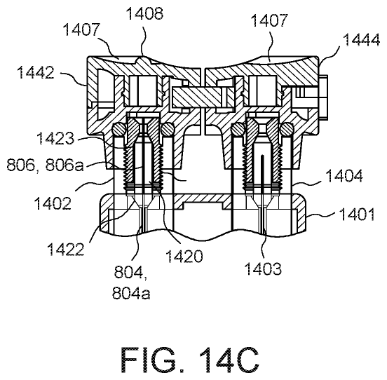

In a further example, an interventional tool 502 may be configured, as shown in FIG. 8R, such that each of the proximal element lines 804a, 804b may be releasably engaged with structures that are activated by removal of the actuator rod 810 that passes through the coupling member 530 and the shaft 900 such that release of the proximal element lines 804a, 804b occurs concurrently with the release of the fixation device 500 from the delivery system 1200. Thus, in some examples, which is depicted in FIG. 8R, each of the proximal element lines 804a, 804b may have a first end portion 806a (e.g., proximal end portion), which may be coupled to an actuator of the delivery system handle 1210 (see e.g., FIG. 14C), a second end portion 806b (e.g., distal end portion) which may be releasably engages to the shaft 900 via the actuator rod 900, and an intermediate portion which may be coupled to the proximal element 610. As described above and as illustrated in FIG. 8A, the stud 540 may be releasably attached to the actuator rod 810 which passes through the coupling member 530 and the shaft 900 of the delivery system 1200. In this way, the actuator rod 810 is connectable with the fixation device 500 and acts to manipulate the fixation device 500 so as to move it through its various positions, which are described above. After the leaflets LF have been coapted, the actuator rod 810 may be removed proximally from the stud 540 which may thereby also release the coupling member 530 from the shaft 900, as described with respect to FIGS. 8A-8E with respect to the coupling system 5015. This action of the actuator rod 810 may be utilized to release the second end portion 806b of each of the proximal element lines 804a, 804b.

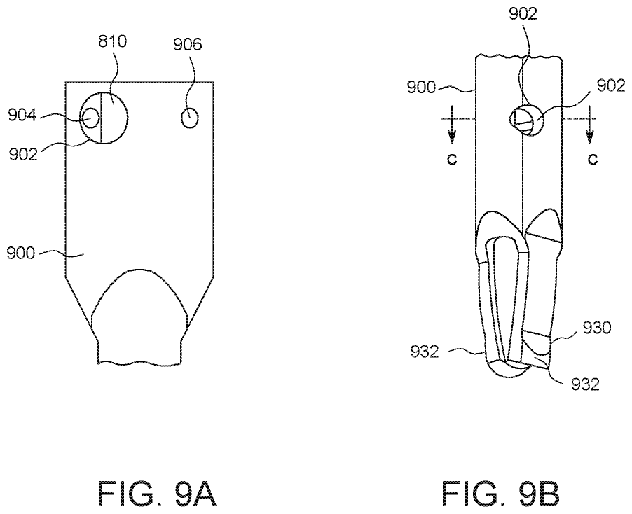

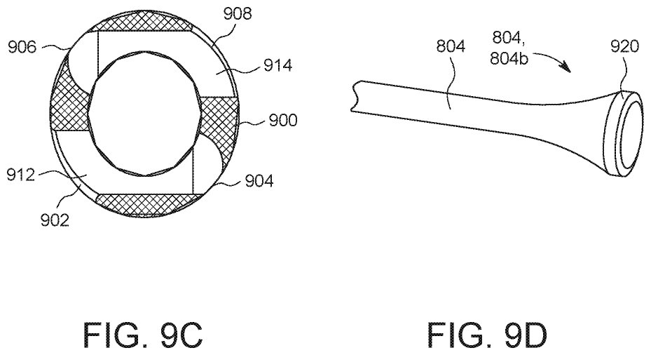

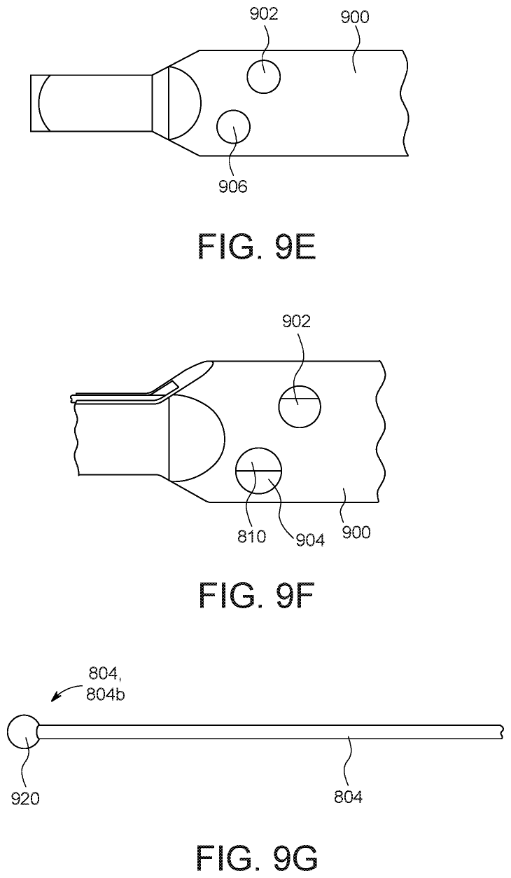

Exemplary features which may be implemented in the interventional tool 502 to facilitate release of the proximal element lines 804a, 804b in this manner are shown in FIGS. 9A-9G. As depicted, an actuator rod 810 may be used as an anchor to restrict proximal movement of one or more proximal element lines 601. The proximal element line 804 has a distal end portion 603b which may include a catch element 920, for example a trumpet 920 having a cone shape (see FIG. 9D) or other shapes, such as a ball 920 having a spherical shape (see FIG. 9G), which can be sized to be received within the shaft 900. As shown in the example of FIG. 9B, the shaft 900 may have spring arms 930 like that of the coupling system 5015 of FIGS. 8D and 8E for releasing the fixation device 500 from the shaft 900. However, the shaft 900 may also have mating elements 932 of FIGS. 8A and 8C. In any event, a portion of the shaft 900 proximal of the spring arms 930 (or the mating elements 932), may have two slots 912 and 914 defined therein. A slot 912 can define holes 902 and 904 and a slot 914 can define holes 908 and 906. The holes 904 and 908 can be sized to receive the catch element 920 of a pair of the proximal element lines 804, respectively, therethrough and into the slots 912 and 914, respectively. The holes 904 and 906 can be sized to prevent the catch element 920 of the proximal element lines 804, respectively, from extending beyond the slots 912 and 914, respectively. The configuration of the slots 912 and 914 and the holes 902-908 can allow for easier manufacture of the features in the shaft 900. The slots 912 and 914 can be drilled to ensure that the slots 912 and 914 do not pass the entire way through the shaft 900. In this example configuration, catch elements 920 of the proximal element lines 804 can be maintained within the shaft 900 to manage the slack of the proximal element lines 804.

In some examples, the catch element 920 of the proximal element line 804 can be inserted into the slot 912 through a hole 902 beyond a longitudinal axis of the shaft 900 and toward the hole 904, and the catch element 920 of the proximal element line 804 can be inserted into the slot 914 through the hole 908 beyond the longitudinal axis of the shaft 900 and toward the hole 906 prior to the insertion and coupling of the actuator rod 810 (which passes through the shaft 900) with the stud 540 of the fixation device 500.

With the actuator rod 810 extending through the shaft 900, the actuator rod 810 may directly engage the catch elements 920 of the proximal element lines 804 thereby preventing their movement back out along the path through which they were inserted. For example, trumpets 920 can be inhibited from being advanced through the holes 908 and 906, respectively, and can be prevented from being pulled past the actuator rod 810 and through the holes 902 and 908, respectively. Accordingly, the second end portions 804b of the proximal element lines 804 can be held in place relative to a shaft 900. Once the actuator rod 810 is decoupled from the stud 540 and subsequently retracted, movement of the catch elements 920 at the distal end portions of the proximal element lines 804 is no longer restricted and the proximal element lines 804 are free to move. Upon proximal retraction, the proximal element lines 804 can thread through the holes 902 and 908, respectively, and decouple from the proximal elements 610.

In accordance with some examples of the disclosed subject matter, the slots 912 and 914 can be drilled at an angle towards the distal end of the shaft 900 (see FIGS. 9E and 9F), e.g., with a hole 904 formed distal to the hole 902 on one side, and the hole 906 formed distal to the hole 908 on the other side. This example configuration of the slots 912 and 914 can provide easier deployment of the proximal element lines 804 and can reduce friction.

Prior to securing the second end portion 804b of each proximal element line 804 with the shaft 900, each proximal element line 804 can be coupled with the respective proximal element 610, such as in the manner described above with respect to FIG. 8R. Thus, when the proximal element lines 804 are actuated proximally, the proximal element lines 804 can move the proximal elements 610 relative to the distal elements 700, thereby moving the proximal elements 610 between their respective raised and lowered positions.

As mentioned above, the fixation device 500 optionally includes a lock 1000 for locking the fixation device 500 in a particular position, such as in any one of the aforementioned open, closed, and inverted positions or any position therebetween.

According to various examples, the lock 1000 may be configured for both locking and unlocking which correspondingly allows the fixation device 500 to be both locked and unlocked. As described in more detail below with respect to various lock examples, such locks may have components disposed between the coupling member 530 and the base 532 which may be configured to selectively arrest proximal-distal movement of the stud 540/the base 532 which consequently arrests movement of the distal elements 700. Such locks may help provide end user control of the final arm angle of the fixation device 500 for tailored and optimal results for each patient. Additionally, such locks may bring the leaflets LF and annulus AN together which may result in beneficial dimensional changes of the target valve which can prevent adverse remodeling of the heart H, particularly for patients with heart failure.

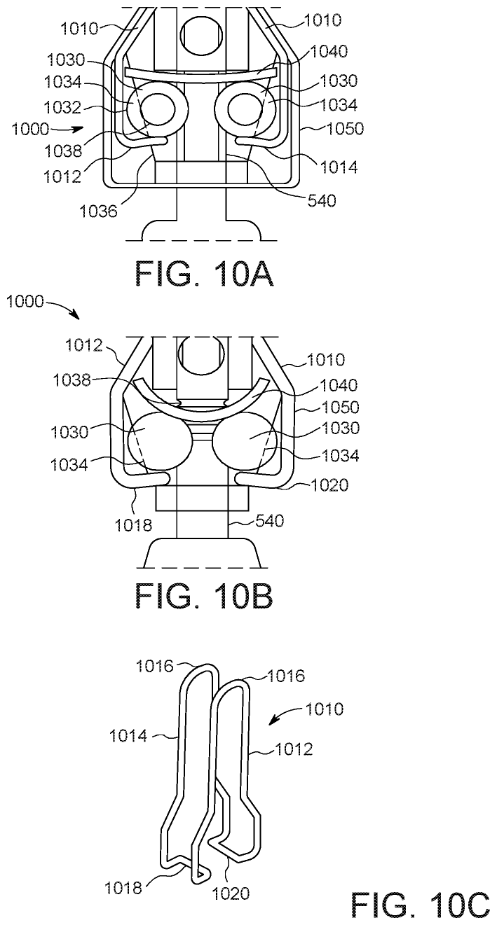

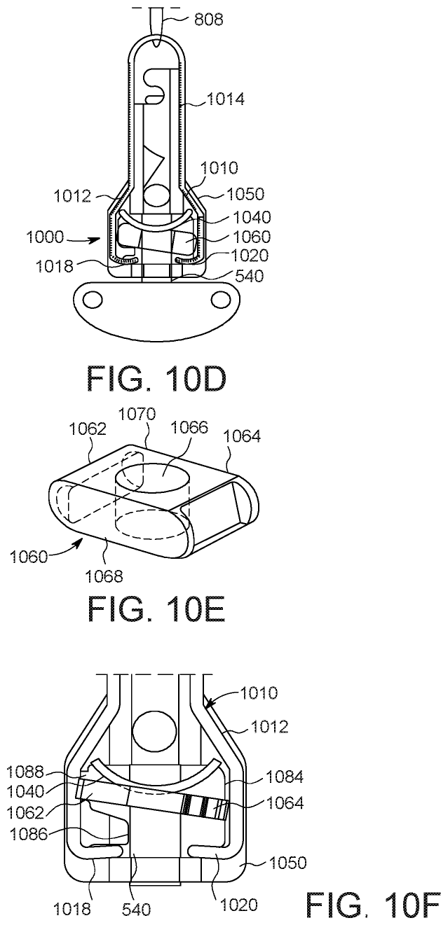

FIGS. 8P-8R illustrate an implementation of the lock 1000. The lock 1000 generally includes a housing 1050, one or more wedging elements 1030, a release harness 1010, and a biasing member 1040. The housing 1050 may be positioned distal to the coupling member 530 and may be free-floating, coupled to, or integral with the coupling member 530, such as at a distal end thereof. The housing 1050 may form a window 1036 which may be defined at opposite sides with sloping surfaces 1034 which slope inwardly toward the stud 540 in a proximal to distal direction. The wedging elements 1030 may be in the form of rolling elements, such as a pair of barbells, disposed on opposite sides of the stud 540 and between the sloping surfaces 1034, as shown in FIGS. 10A and 10B.

Each barbell 1030 may have a pair of generally cylindrical caps 1032 and a shaft 1038 therebetween. The barbells 1030 and the stud 540 are preferably comprised of cobalt chromium or stainless steel, however any suitable material may be used. The biasing member 1040 may be a spring, such as a leaf spring, for example, and may be positioned at a proximal end of the housing 1050 between the sloping surfaces 1034 and proximal to the barbells 1030 such that the spring 1040 bears on the barbells 1030 and biases them in a distal direction. Thus, when the barbells 1030 are pushed distally by the spring 1040, they are correspondingly pushed inwardly and wedged against the stud 540 by the sloping surfaces 1034, as illustrated by FIG. 10A, which depicts the barbells 1030 in a proximal and unlocked position, and FIG. 10B, which depicts the barbells 1030 in a distal and locked position.

As shown in FIGS. 8P, 8Q, and 10C, the release harness 1010 may be in the form of a ridged wire or rod that may extend proximally from the stud 540 toward a proximal end of the fixation device 500 and at opposite sides thereof. In this regard, the release harness 1010 may form a first portion or front portion 1012 and a second portion or rear portion 1014. Each of the first portion 1012 and the second portion 1014 may include a crest or closed proximal end 594 through which a lock line 808 may be threaded and engaged, as described below. The release harness 1010 may also form hooked distal ends 1018, 1020 which may extend between the first portion 1012 and the second portion 1014 and between the sloping surfaces 1034 and the stud 540, as shown in FIGS. 10A and 10B. Thus, the hooked ends 1018 and 1020 may be moveable proximally-distally within the window 1036 formed between the sloped surfaces 1034 and the stud 540. Additionally, the hooked ends 1018 and 1020 may be positioned distal of the barbells 1030 such that pulling up on the harness 1010 moves the hooked ends 1018, 1020 proximally so as to push the respective barbells 1030 against the bias of the spring 1040 and move them to their unlocked position.

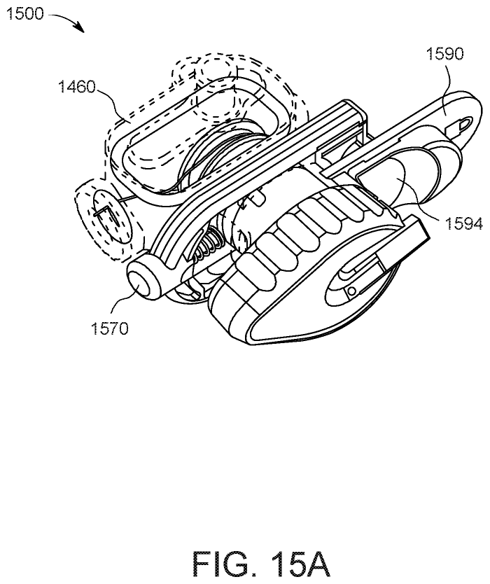

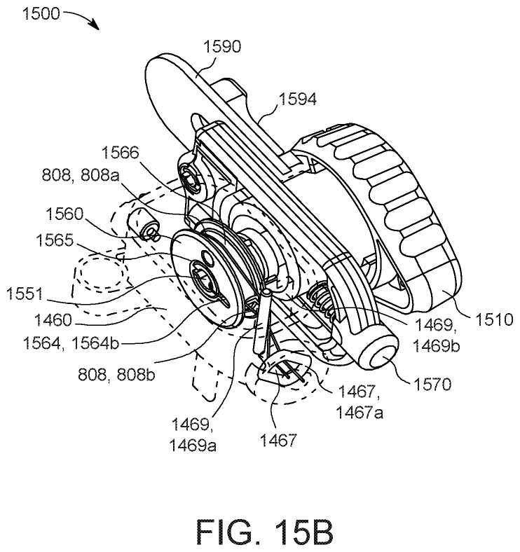

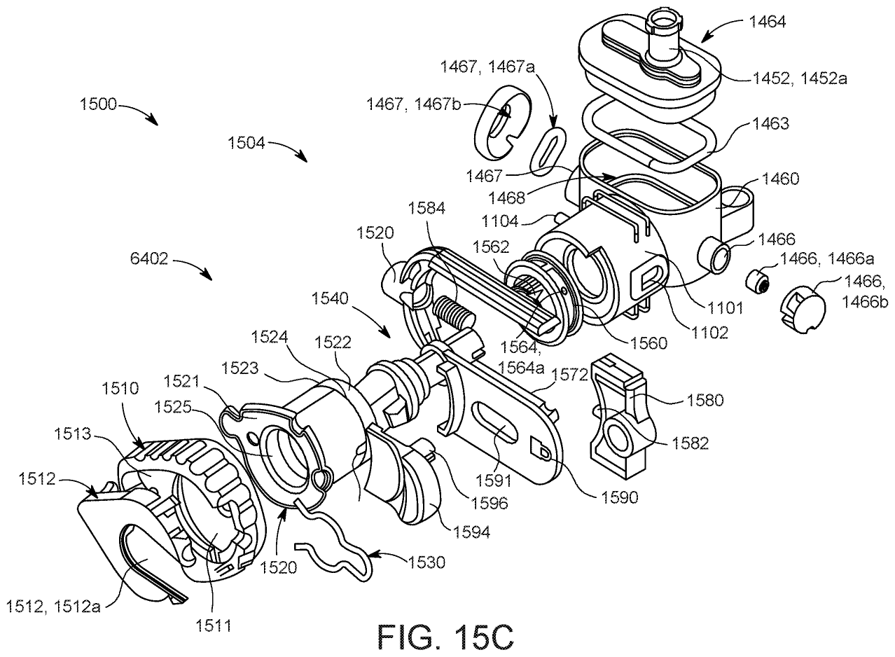

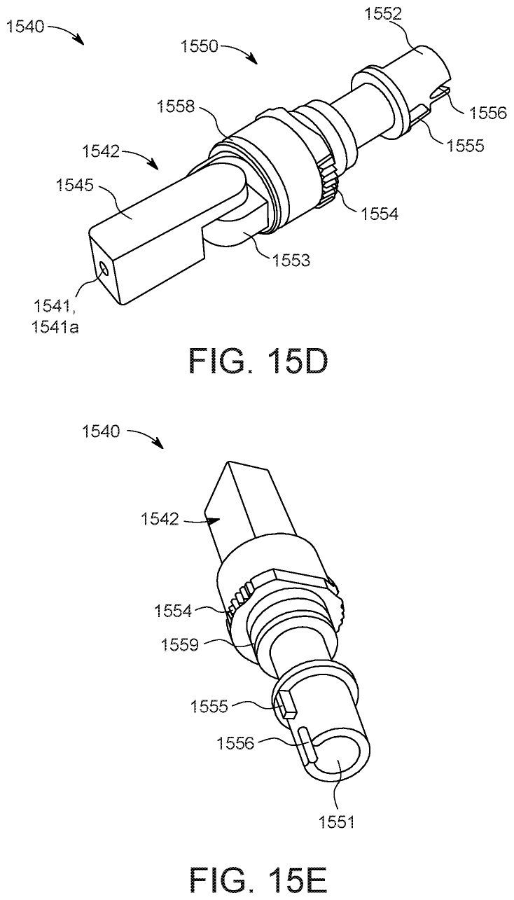

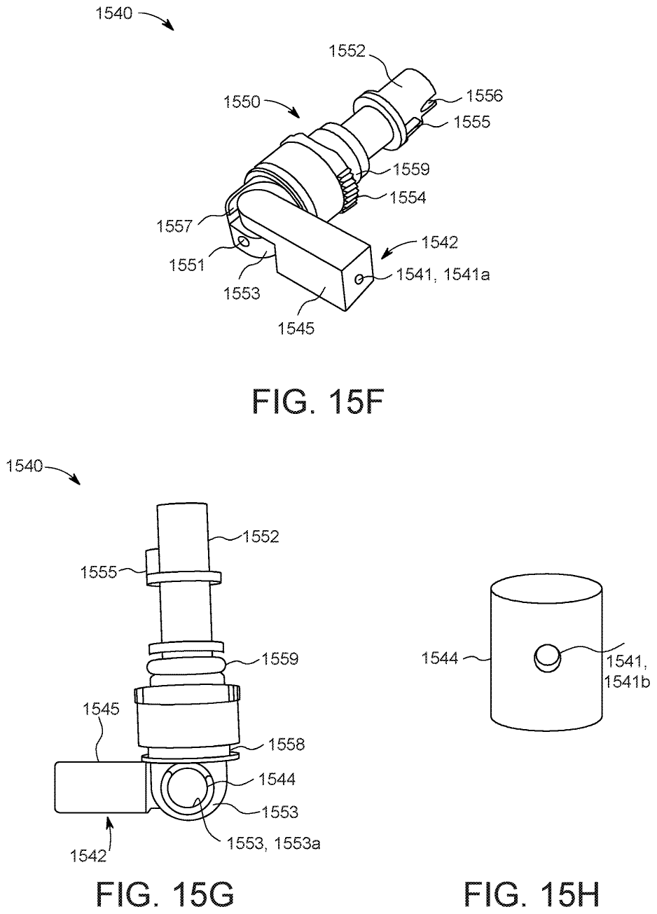

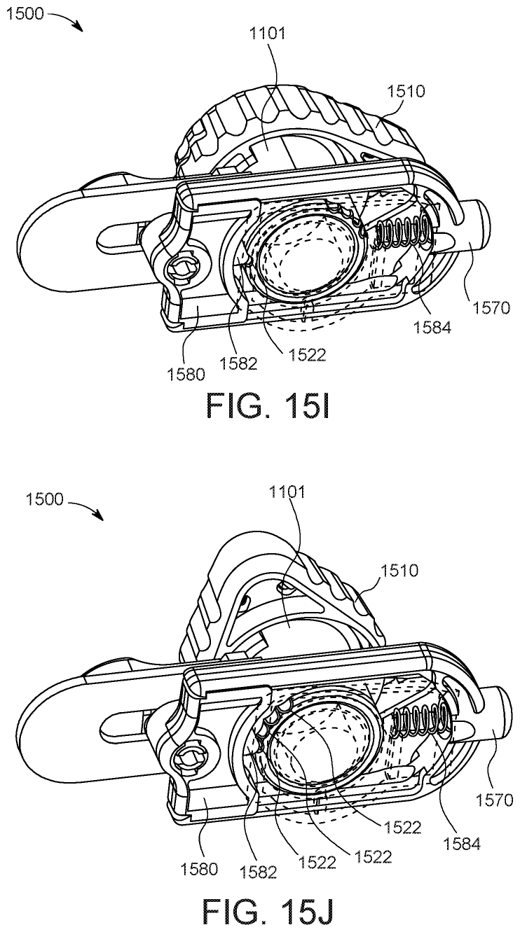

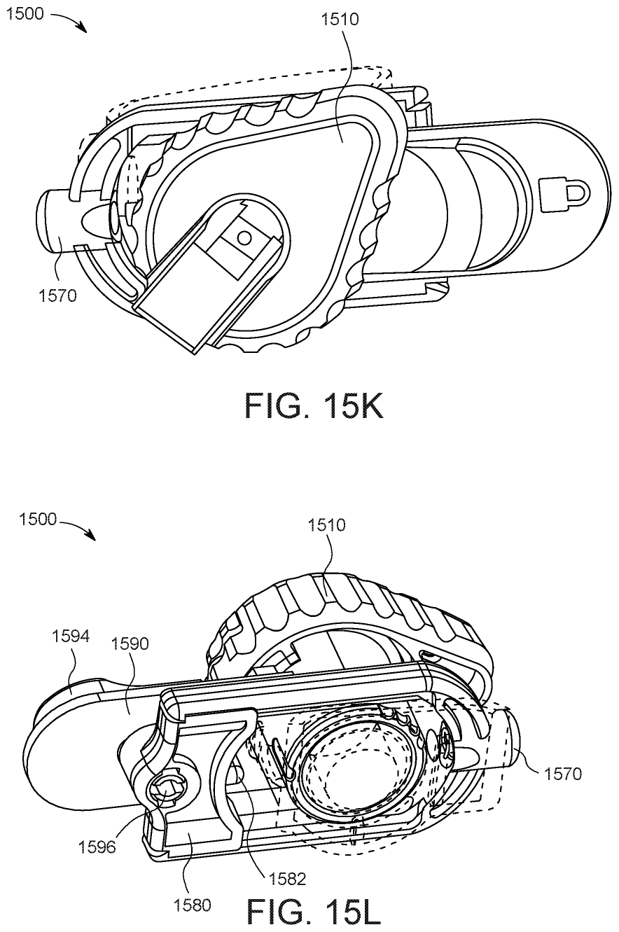

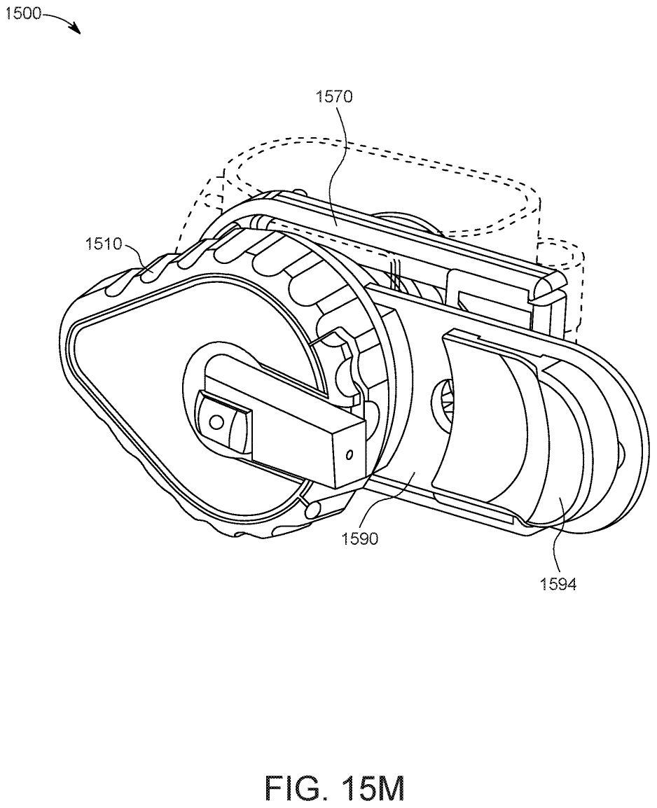

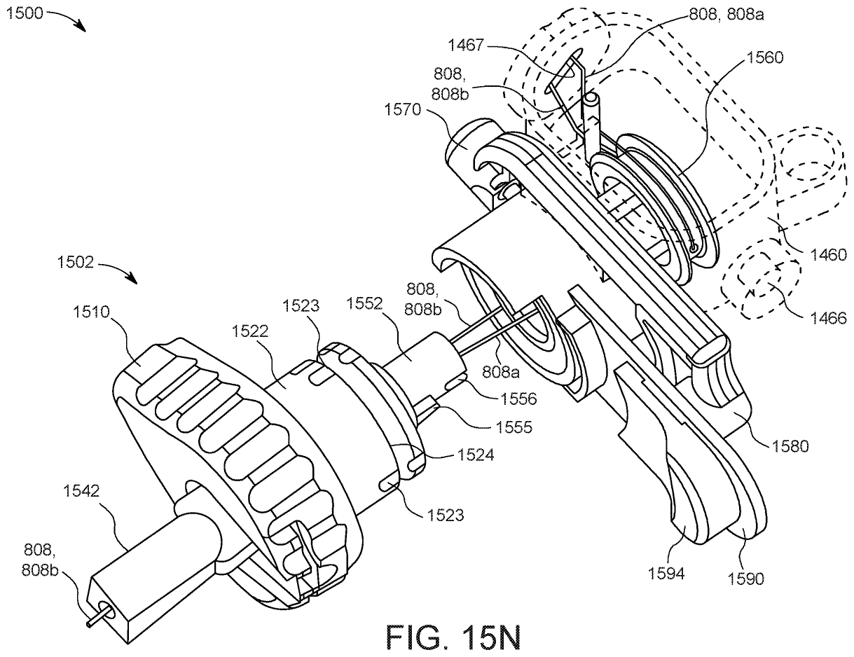

Movement of the harness 1010 may be performed by one or more lock line 808 which may be coupled to the harness 1010 by such as by threading the lock line 808 through and engaging one or more of the proximal ends 594 of the first portion 1012 and the second portion 1014 thereof, as shown in FIGS. 8P and 8Q. Such lock line 808 may have a first end 808a fixedly secured to the delivery system handle 1210 of the delivery system 1200 and a second end 808b releasably secured to the delivery system handle 1210 (see e.g., FIG. 15N), as described in more detail below. In this regard, tension can be selectively applied to the lock line 808 to unlock and lock the lock 1000. Also, the lock line 808 can be released from the release harness 1010 prior to, concurrently with, or after release of the fixation device 500 from the delivery system 1200 which may be achieved by releasing the second end 808b from the delivery system handle 1210 and pulling the lock line 808 and its second end 808b through the shaft 810. The lock line 808 may be comprised of any suitable material, typically wire, nitinol wire, cable, suture, or thread, to name a few. In addition, the lock line 808 may include a coating, such as parylene. Parylene is a vapor deposited pinhole free protective film which is conformal and biocompatible. It is inert and protects against moisture, chemicals, and electrical charge.

When an upwards force is applied to the harness 1010 by the lock line 808, the hooked ends 1018, 1020 may raise the barbells 1030 against the spring 1040, as shown in FIG. 10A. This may draw the barbells 1030 up along the sloping surface 1034 which unwedges the barbells 1030 from against the stud 540. In this position, the stud 540 is free to move. Thus, when the lock line 808 is tensioned to raise or lift the harness 1010, the lock 1000 is in an unlocked position wherein the stud 540 is free to move the actuator 520 and therefore the distal elements 700 to any desired position. Releasing tension in the lock line 808 may, on the other hand, transition the lock 1000 to a locked position, as shown in FIG. 10B. Thus, by releasing the upwards force on the barbells 1030 by the hooked ends 1012, 1014, the spring 1040 forces the barbells 1030 downwards and wedges the barbells 1030 between the sloping surface 1034 and the stud 540. This restricts motion of the stud 540, which in turn locks the actuator 520 and therefore the distal elements 700 in place. In addition, the stud 540 may include one or more grooves or indentations 1038 which may receive the shaft 1038 of each barbell 1030. This may provide more rapid and positive locking by causing the barbells 1030 to settle in a definite position, increase the stability of the lock 1000 by further preventing movement of the barbells 1030, as well as tangible indication to the user that each barbell 1030 has reached a locking position. In addition, the grooves 1038 may be used to indicate the relative position of the distal elements 700, particularly the distance between the distal elements 700. For example, each groove 1038 may be positioned to correspond with a 0.5-or 1.0-mm decrease in distance between the distal elements 700. As the stud 540 is moved, the barbells 1030 may contact the grooves 1038, and by counting the number of grooves 1038 that are felt as the stud 540 is moved, the user can determine the distance between the distal elements 700 and can provide the desired degree of coaptation based upon leaflet thickness, geometry, spacing, blood flow dynamics and other factors. Thus, the grooves 1038 may provide tactile feedback to the user.

The lock 1000 allows the fixation device 500 to remain in an unlocked position when attached to the delivery system 1200 during grasping and repositioning and then maintain a locked position when left behind as an implant. However, the lock 1000 may be repeatedly locked and unlocked throughout the placement of the fixation device 500 if desired. Once the final placement is determined, the lock line 808 may be removed and the fixation device 500 may be left behind.

FIGS. 10D-10F depict a lock 1000 according to another implementation of the present disclosure that may be incorporated into the fixation device 500 of the disclosure. In this implementation, the lock 1000 also includes a housing 1050, a spring 1040, a release harness 1010, and a wedging element 1060. However, instead of the sloping surfaces 1034 as present in the example of the locking element 1000, the housing 1050 may include parallel sidewalls 1084 and may include a finger or protrusion 1086 extending from one of the sidewalls 1084 toward a stud 540. Such finger 1086 may slope in a distal direction and may define a proximal notch 1088. Also, as shown in FIG. 10F, a first hooked end 1018 of the release harness 1010 may be positioned distal of the finger 1086, Furthermore, the wedging element 1060 may include a binding lever or binding plate 1060. As shown in FIG. 10E, the binding plate 1060 may have an oblong shape that may extend lengthwise between a first end 1062 and a second end 1064 thereof. An aperture 1066 may be formed between the first end 1062 and the second end 1064 and may extend from a top planar surface 1070 through a bottom planar surface 1068 of the binding plate 1060. The binding plate 1060 may be positioned between the sidewalls 1068 so that the stud 540 passes through the aperture 1066 and so that the first end 1062 of the binding plate 1060 is positioned within the notch 1088 proximal of the finger 1086, as best shown in FIG. 10F. Thus, the finger 1086 may be positioned between the first end 1062 of the binding plate 1060 and the first hooked end 1018 of the released harness 1010. Also, the spring 1040 may be positioned proximal to the binding plate 1060 and provide downward or distal bias thereto. The binding plate 1060 and the stud 540 may be comprised of any suitable material. In some implementations, the binding plate 1060 may have a higher hardness than the stud 540. In other implementations, the binding plate 1060 may be comprised of a flexible or semi-flexible material. Such flexibility may allow slight movement of the stud 540 in the proximal and distal directions, therefore allowing slight movement of the distal elements 700 when the lock 1000 is in the locked position. This may allow the fixation device 500 to adjust in response to dynamic cardiac forces.

FIGS. 10E and 10F illustrate the binding plate 1060 in a locked position or configuration. In this regard, the spring 1040 pushes the binding plate 1060 in a distal direction. However, because the first end 1062 of the binding plate 1062 is positioned within the notch 1088, axial movement of the first end 1062 toward a distal end of the housing 1050 is prohibited while axial movement of the second end 1064 of the binding plate 1060 is permitted. Thus, the finger 1086 obstructs the first end 1062 from axial movement and creates a lever type movement of the binding plate 1060. Moreover, the finger 1086 obstructs the first hooked end 1018 of the release harness 1010 from axial movement resulting in a side-to-side pivoting of the release harness 1010 upon tension of the lock line 808. This pivoting movement correspondingly results in a second hooked end 1020 of the release harness 1010 moving proximally and controlling movement of the second end 1064 of the binding plate 1060. As such, when an upwards force is applied to the harness 1010 by the lock line 808, the second hooked end 1020 of the release harness 1010 raises the second end 1064 of the plate 1060 against the spring 1040 so that the planar surfaces 1068, 1070 of the binding plate 1060 become oriented substantially perpendicular to the stud 540. This aligns the aperture 1066 with the stud 540 allowing free movement of the stud 540 in the proximal-distal direction. Thus, in this state, the lock 1000 is unlocked wherein the stud 540 is free to move the actuator 520 and therefore the distal elements 700 to any desired position.

Release of the harness 1010 by the lock line 808 transitions the lock 1000 back to the locked position. By releasing the upwards force on the second end 1064 of the binding plate 1060, the spring 1040 forces the second end 1064 of the binding plate 1060 downwards, which misaligns the aperture 1066 relative to the stud 540, and correspondingly wedges the binding plate 1060 against the stud 540, as best shown in FIG. 10F. This arrests movement of the stud 540, which in turn locks the actuator 520 and therefore the distal elements 700 in place. It may be appreciated that the binding plate 1060 may have any suitable form to function as described above. For example, the plate 1060 may have a variety of shapes with or without the planar surfaces 1068, 1070 and/or the aperture 1066 may be of a variety of shapes and positioned in a variety of locations, to name a few. For example, the binding plate 1060 may not have a through-hole, like that of the aperture 1066, but may rather have a notch such that the binding plate 1060 does not encircle the stud 540 but rather partially surrounds it. Further, any number of binding plates 1060 may be present. Each binding plate 1060, in this regard, may provide an additional binding location which may enhance lock performance.

While the above-described nonlimiting examples of the fixation device 500 may utilize a push-to-open, pull-to-close mechanism for opening and closing the distal elements 700, a pull-to-open, push-to-close mechanism may alternatively be provided.

For example, the distal elements 700 may be coupled at their proximal ends to the stud 540 rather than to the coupling member 530, and the legs 510 may be coupled at their proximal ends to the coupling member 530 rather than to the stud 540. In this example, when the stud 540 is pushed distally relative to the coupling member 530, the distal elements 700 may close, while pulling on the stud 540 proximally toward the coupling member 530 may open the distal elements 700. Regardless, the aforementioned lock examples may be configured to arrest the stud 540 to lock the distal elements 700 in the desired position, as described.

The fixation devices and components thereof described above are provided as examples are not to be considered as limiting to fixation devices suitable for use with other aspects of the disclosure.

FIG. 11 depicts various components of an exemplary interventional system 1100 which may be utilized to implant the fixation device 500 within a target valve. Such components may include the delivery system 1200, the steerable guide system 1105, and the stabilizer 1900. As described in more detail below, the delivery system 1200 may generally include the delivery catheter handle 1210 and the delivery catheter 850. The delivery catheter handle 1210 may include a plurality of controls for steering the fixation device 500 to a target valve and for controlling various features of the fixation device 500, such as the distal elements 700 and the proximal elements 610, and for releasing the fixation device 500 from the delivery catheter 850, for example. Additionally, the steerable guide system 1105 may generally include a plurality of guide catheter assemblies 1700, 1800 which may be configured to provide a conduit to help guide the delivery catheter 850 and the fixation device 500 to a target valve, such as the mitral valve MV or the tricuspid valve TV, for example, and for optimal positioning of the same above a valve plane of the target valve. The stabilizer 1900 may support the delivery system 1200 and the steerable guide system 1105 and may facilitate stable and controlled movement of the fixation device 500 in-situ.

FIGS. 12A-18C depict the delivery system 1200 according to an implementation of the present disclosure. The delivery system 1200 may include the delivery catheter handle 1210 and the delivery catheter 850 which may extend distally from the delivery catheter handle 1210. The delivery system 1200 may also include the actuator rod 810, one or more proximal element lines 501, such as the proximal element lines 804a and 804b, and one or more lock lines 808 which may extend from respective controls in the handle 1210 and through the delivery catheter 850 to the fixation device 500 coupled to the delivery catheter 850, as shown in FIG. 12B and as described in more detail below.

According to some examples of the delivery catheter handle 1210, the delivery catheter handle 1210 may include a housing 1212, a positioner assembly 1300, a gripper control assembly 1400, a fluid management assembly 1450, a lock control assembly 1500, and a delivery catheter fastening assembly 1600. The actuator rod 810 may extend from the positioner assembly 1300, and the positioner assembly 1300 may be configured to actuate the actuator rod 810 in a proximal-distal direction for moving the distal elements 700 between open, closed, and inverted positions, examples of which are described above. The positioner assembly 1300 may also be configured to release the actuator rod 810 from the fixation device 500 for deployment of the fixation device 500. The delivery device handle 1210 may also include a deployment control system 1370 that may be coupled to the positioner assembly 1300 for prevention of inadvertent deployment of the fixation device 500 until desired. The proximal element lines 804a, 804b may extend from the gripper control assembly 1400, and the gripper control assembly 1400 may be configured to actuate the proximal elements lines 804a, 804b to move the proximal elements 610 between their raised and lowered positions, as described above. The lock line 808 may extend from the lock control assembly 1500, and the lock control assembly 1500 may be configured to actuate the lock line 808 and correspondingly the lock 1000 of the fixation device 500 between the locked and unlocked configuration, as described above. Additionally, the lock control assembly 1500 may be configured to release the lock line 808 from the fixation device 500. The actuator rod 810, the proximal element lines 804a, 804b, and the lock line 808 may be directed into the fluid management assembly 1450 which itself may be coupled to the delivery catheter 850 for providing sterile fluid through one or more lumens of the delivery catheter 850. During a transcatheter procedure, it may be desirable to advance or retract the delivery catheter 850 relative to the steerable guide system 1105 to help position the fixation device 500 within a target valve. The delivery catheter fastening assembly 1600 may be configured to selectively secure and unsecure the delivery catheter 850 so that it can be freely advanced or retracted relative to the steerable guide system 1105 and secured in a desired position so that the delivery catheter 850 is constrained from axially translating relative to the steerable guide system 1105. Each of these features are described in more detail below.

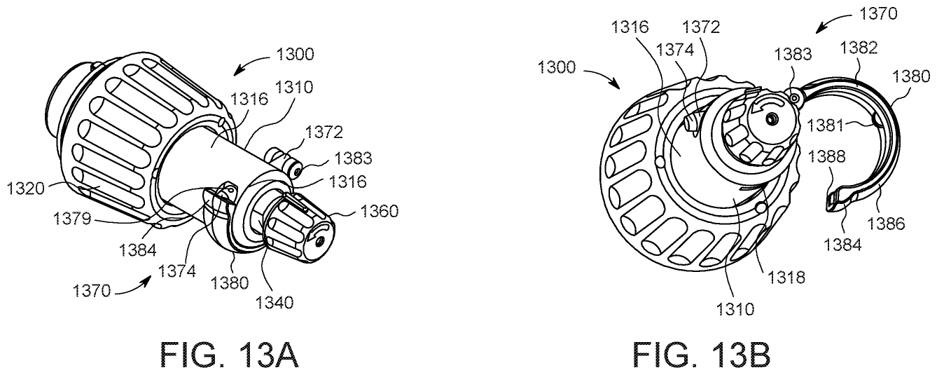

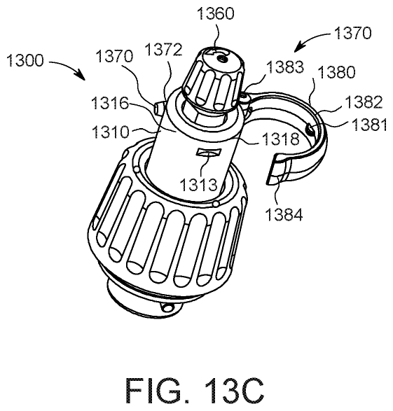

FIGS. 12B-13B depict the positioner assembly 1300 according to an implementation of the present disclosure. In some examples, the positioner assembly 1300 may generally include a slider 1310, an actuator knob 1320, one or more guide pins 1330, an actuator shaft 1340, a bearing 1350, and an actuator rod handle 1360.

According to various examples, the slider 1310 may be substantially cylindrical and may have an axial channel 1314 that extends entirely along its length from a proximal end to a distal end thereof. A transverse opening 1311 may extend through the slider 1310 near its proximal end such that it extends through opposing sides of the slider 1310 and intersects the channel 1314. In some examples, the transverse opening 1311 is offset relative to a longitudinal axis of the slider 1310. Nonetheless, in such implementation, the transverse opening 1311 may be oriented generally perpendicular (+/−5 degrees) relative to the longitudinal axis of the slider 1310. Also at the proximal end of the slider 1310, a notch 1313 may extend radially inwardly into the slider 1310. Such transverse opening 1311 may be configured to releasably receive a deployment pin of a deployment system, and the notch 1313 may be configured to receive a protuberance of such deployment system, as described further below. At a distal end of the slider 1310 in some examples, a pair of the guide pins 1330 may extend radially outwardly therefrom and may correspondingly engage grooves 1214 within the delivery handle housing 1212. This pin 1330 and groove 1214 engagement may rotationally constrain the slider 1310 relative to the housing 1212 while permitting translation along the housing 1212. Such guide pins 1330 may, in some examples, be orthogonal to the longitudinal axis of the slider 1310 and may be press-fit to the slider 1310, threaded to the slider 1310, or integral with the slider 1310, for example. The slider 1310 may also include external threads 1312 that extend helically along an outer surfaces thereof and that extend along at least a portion of its length, as best shown in FIG. 12C. In this regard, the threads 1312 may extend along a length of the slider 1310 disposed between the transverse opening 1311 and the guide pins 1330.

The actuator knob 1320 may be annular such that it has a generally cylindrical exterior and interior. However, the actuator knob 1320 can have other shapes, such as a conical shape, for example. As shown in FIG. 12C, in some examples, the actuator knob 1320 may extend circumferentially about the slider 1310 and may have internal threads 1322 that engage the external threads 1312 of the slider 1310 such that rotating the actuator knob 1320 in a first direction translates the slider 1310 in a proximal direction, and rotating the actuator knob 1320 in a second direction translates the slider 1310 in a distal direction. The actuator knob 1320 may be rotatably connected to a proximal end of the delivery handle housing 1212 while being translationally constrained. For example, the actuator knob 1320 may include a circumferential lip 1324 that defines a circumferential groove that receives a corresponding lip 1218 of the housing 1212, as shown in FIG. 12C. Thus, with the actuator knob 1320 translationally constrained, and the slider 1310 rotationally constrained via the pins 1330 and the grooves 1214, the slider 1310 is free to translate relative to the actuator knob 1320 upon its rotation.

According to some examples, the actuator shaft 1340 may be positioned within the axial channel 1314 of the slider 1310, as shown in FIG. 12C, and may be rotatable and slidable therein. The actuator shaft 1340 may be connected directly or indirectly to a proximal end of the actuator rod 810. In this regard, the actuator shaft 1340 may include a connection feature for connecting to the actuator rod 810 in a secure manner such that it prevents relative rotation between the actuator shaft 1340 and the actuator rod 810. For example, the actuator shaft 1340 may have a central opening that receives the proximal end of the actuator rod 810 and a collet 1342 disposed within the central opening. Such collet 1342 may be configured to grasp or otherwise engage and secure the actuator rod 810 to the actuator shaft 1340. For example, the collet 1342 may be threaded to the actuator shaft 1340 and have fingers 1343 that are cammed inwardly by sloping inner surfaces 1341 of the actuator shaft 1340 as the collet 1342 is advanced distally within the central opening. Alternative connection features are also contemplated, such as a crimp ring and direct threaded engagement between actuator shaft and actuator rod, for example.

The actuator shaft 1340 may also have a proximal portion 1345, a distal portion 1349, and an intermediate portion 1347. In some examples, the actuator rod handle 1360 may be connected to the proximal portion 1345 to help facilitate manual manipulation of the actuator rod 810 when desired to release the fixation device 500. In this regard, the proximal portion 1345 may be positioned at least partially outside of the axial channel 1314 of the slider 1310. On the other hand, the intermediate portion 1347 may be at least partially disposed within the channel 1314. The intermediate portion 1347 may have a larger cross-sectional dimension than the proximal and distal portions 1345, 1349 and may include a notch 1344 extending radially inwardly. Such notch 1344 may be configured to align with the transverse opening 1311 of the slider 1310, as shown in FIG. 12C, so that a deployment pin 1372 (FIGS. 13A-13C) extending through the transverse opening 1311 and the notch 1344 prevents the actuator shaft 1340 from rotating and translating relative to the slider 1310, as discussed in more detail further below.

In various examples, the bearing 1350 may be disposed within the axial channel 1314 of the slider 1310, and the distal portion 1349 of the actuator shaft 1340 may be removably received within the bearing 1350, as shown in FIG. 12C. The bearing 1350 may be secured to the slider 1310 via a press-fit or threaded connection, for example. The bearing 1350 may be a one-way bearing such that, when the distal portion 1349 of the actuator shaft 1340 is received within the bearing 1350, the actuator shaft 1340 is rotatable in a first direction but prevented from rotation in a second direction, for example. As mentioned above, in some examples, the actuator rod 810 may be released from the fixation device 500 by rotating the actuator rod 810 in the first direction which may unthread a distal end of the actuator rod 810 from the stud 540 or the base 532 of the fixation device 500. Thus, in some examples, the bearing 1350 may allow the actuator rod 810 to be rotated for release but prevent rotation in the opposite direction which may help prevent inadvertent overtightening. An exemplary positioner assembly with one-way bearing is disclosed in U.S. Pat. No. 10,660,625, the disclosure of which is hereby incorporated by reference herein in its entirety.

The actuator shaft 1340 may be coupled to the slider 1310 via the deployment system 1370, for example. An exemplary deployment system 1370 is depicted in FIGS. 12C-13C and may include one or more of the transverse opening 1311 of the slider 1310, the notch 1344 of the actuator shaft 1340, and the deployment pin 1372 which may be releasable received within the transverse opening 1311 and the notch 1344. The deployment system 1370 may also include a handle 1380. The handle 1380 may connect to the deployment pin 1372 via a hinge 1383 in some examples. In some examples, the handle 1380 may be pivotable relative to the slider 1310 when the pin 1372 is disposed therein so as to pivot between a locked configuration (shown in FIG. 13A) and an unlocked configuration (shown in FIGS. 13B and 13C). In the depicted implementation, the handle 1380 may extend around a portion of the slider 1310 (e.g., about substantially one half of the slider 1310 or more). The handle 1380 may, for example, include a curved profile 1382 that corresponds to a curved profile 1316 of the slider 1310, which advantageously minimizes a profile of the deployment system 1370 in the engaged configuration. This permits removal of the pin 1372 when the handle 1380 is rotated to the unlocked configuration and prevents removal of the pin 1372 when the handle 1380 is in the locked configuration. The handle 1380 may, for example, include at least one protuberance 1381 extending from an interior surface 1382 of the handle 1380 which may cooperate with the indentation or notch 1313 in the slider 1310. The protuberance 1381 and the notch 1313 can prevent the handle 1380 from inadvertently rotating off of the slider 1310. The handle 1380 may also include a pin cover 1384 in some examples.

The pin cover 1384 may be configured to cover and retain a pin tip 1374 when the handle 1380 is in the locked configuration, which can in turn retain the pin 1372 within the slider 1310, as shown in the example of FIG. 13A. The pin cover 1384 may have a groove 1388 which can provide a space for the pin tip 1374. When provided, such covering 1384 helps prevent inadvertent contact with the pin 1372 that could result in premature disengagement. A spring biased ball 1379 may also be included in the pin 1372 to help further secure the pin 1372 from being inadvertently removed from the slider 1310 in some examples.

In operation, the positioner assembly 1300 may be utilized to move the distal elements 700 between the open, closed, and inverted positions. In some examples, rotating the actuator knob 1320 in the first direction may translate the slider 1310 and consequently the actuator rod 810 proximally which may move the distal elements 700 from the closed to open position and from the open position to the inverted position. On the other hand, rotating the actuator knob 1320 in the second direction may advance the slider 1310 and consequently the actuator rod 810 distally which may move the distal elements 700 from the inverted position to the open position and from the open position to the closed position. When it has been determined that the valve leaflets LF have been sufficiently grasped by the fixation device 500, the fixation device 500 may be released from the delivery catheter 850. In some examples, this may be achieved by releasing the handle 1380 from the slider 1310 and removing the pin 1372 from the slider 1310 which frees the actuator shaft 1340 from the slider 1310. Once the pin 1372 has been removed, the actuator shaft 1340 and the actuator rod 810 may be rotated via the actuator handle 1360 in the first direction thereof to thereby release the actuator rod 810 from the stud 540 or the base 532. Once released, the actuator rod 810 can be pulled proximally relative to the slider 1310 which retracts the actuator rod 810 and positions it proximal to the coupling interface between the shaft 900 and the coupling member 530, as described above with respect to the coupling system 800 of FIGS. 5O and 5P and also similarly described with respect to the coupling system 6015 of FIGS. 5Q and 5R. Other exemplary deployment control systems and positioner assemblies that may be used in the delivery system 1200 are disclosed in U.S. Pub. No. 2021/0353419 (“the '419 Publication”), the disclosure of which is hereby incorporated by reference herein in its entirety.

FIGS. 12B and 14A-14C depict the gripper control assembly 1400 according to an implementation of the present disclosure. Examples of the gripper control assembly 1400 may generally include one or more of a first proximal element line handle 1402, a second proximal element line handle 1404, and an interlock 1410 selectively coupling the first and second element line handles 1402, 1404 to each other. In some examples, the first proximal element line 804a may be coupled to the first proximal element line handle 1402, and the second proximal element line 804b may be coupled to the second proximal element line handle 1404. Each of these handles 1402, 1404 may separately and independently actuate the first and second proximal element lines 804a, 804b so as to separately and independently raise and lower the proximal elements 610 between their raised and lowered positions, according to examples of the disclosure. However, when the interlock 1410 couples the first and second element line handles 1402, 1404 together, the first and second proximal element lines 804a, 804b and, consequently, the proximal elements 610 may be actuated concurrently in some examples. In the implementation depicted, the first and second proximal element line handles 1402, 1404 can be aligned in parallel which helps facilitate concurrent actuation. Although two proximal element line handles 1402, 1404 are depicted, one proximal element line handle may be provided where the single proximal element line 804 is coupled to both proximal elements 610, such as in the interventional device 502 arrangement depicted in FIG. 8Q. A third proximal element line handle can also be provided where the fixation device 500 may include a third proximal element 610 in some examples.

The first and second proximal element line handles 1402, 1404 may each include a connection mechanism for connecting to the respective proximal element lines 804a, 804b. In the implementation depicted, each proximal element line handle 1402, 1404 may connect to the proximal end portion 806a (see FIG. 14C) of its respective proximal element line 804a, 804b, while the second end portion 806b of each proximal element line 804a, 804b may be releasably coupled to the shaft 810, such as described with respect to FIGS. 8R and 9A-9F. Thus, in the implementation depicted, the connection mechanism of each proximal element line handle 1402, 1404 may be configured to connect to the first end portion 806a of the respective proximal element line 804a, 804b. For example, the connection mechanism can include a collet 6220 that may securely grasp the proximal end portion 806a of the proximal element lines 804a, 804b.

As shown, the first proximal element line handle 1402 may have a lumen 1403 with a central axis, and a screw 6224 therein. The screw 6224 can have a lumen 1423 with the collet 1420 therein, the collet 1420 may have fingers 1422, for example. The proximal end portion 806a of the first proximal element line 804a can be disposed within a recess defined by the fingers 1422 according to some examples. When the screw 6224 is tightened, the collet fingers 1422 may tighten down on and grasp the proximal element line 804a. In this regard, the proximal element line 804a may be fixed in a tensioned or non-tensioned state. The proximal element line 804b may be coupled to the proximal element line handle 1404 using a similar collet system (i.e., connection mechanism).

Although the depicted implementation includes a connection mechanism including the collet 1420, other connection mechanisms may be utilized for securing a first end 91G of the proximal element line 804a, 804b to the proximal element line handle 1402, 1404, such as a swage or a knot, for example.

One or more of the proximal element line handles 1402 and 1404 may also include respective end caps 1442, 1444 disposed at their proximal ends in some examples. Each of the end caps 1442 and 1444 can, for example, include finger grip portions, such as one or more top concave portions 1407. In various examples, one or more of the end caps 1442 and 1444 may include a bump 1408 which may provide tactile feedback to the surgeon as to which handle 1442, 1444 and corresponding proximal element 610 is being actuated. Other tactile features can be utilized such as protrusion, ridge, or roughened surface, for example.

The interlock 1410 may, for example, be moveable between an unlock position in which the first and second proximal element line handles 1402 and 1404 are independently actuatable and a locked position in which the first and second proximal element line handles 1402 and 1404 are coupled together to be actuatable together. The interlock 1410 can be fixedly or movably coupled to the first proximal element line handle 1402 and releasably coupled to the second proximal element line handle 1404.

The interlock 1410 can include, for example, a latch 1412 and a recess 1416. The latch recess 1416 can receive the latch 1412, such that the proximal element line handles 1402 and 1404 are operably connected for concurrent actuation. In some examples of the unlocked position, the latch 1412 can be removed from the recess 1416, such that the proximal element line handles 1402 and 1404 are not operatively connected and are independently actuatable. The latch 1412 can be configured to be moved linearly, rotationally, or in any suitable motion to lock and unlock the interlock 1410 as described. Also, the latch 1412 and the recess 1416 can have complementary dovetail shapes in various examples. The dovetail shapes can limit the proximal element line handles 1402 and 1404 from pulling away from one another when the latch 1412 is engaged with the recess 1416. The dovetail shapes can also prevent the interlock 1410 from switching between the lock and unlock positions. A locking lever 1414 can be provided to facilitate movement of the latch 1412 in some examples. Actuating the interlock 1410 between the locked position and the unlocked position can, for example, be performed with a single hand or a single finger. Either of the latch 1412 and the recess 1416 can be disposed in either of the first proximal element line handle 1402 and the second proximal element line handle 1404. Other interlock examples that may be used in the gripper control assembly 1400 are disclosed in U.S. Pub. No. 2021/0015614, the disclosure of which is hereby incorporated by reference herein in its entirety.

The proximal element line handles 1402, 1404 may each be moveably coupled to a base 1401 which may be secured to the housing 1212 of the delivery system handle 1210 in some examples. In this regard, each proximal element line handle 1402, 1404 and their respective connection mechanisms may be moveable in a proximal-distal direction between a first position and a second position relative to the base 1401, according to some examples. Some examples of a first position is shown in FIG. 12B and is a tensioned position in which the proximal element lines 804a, 804b are tensioned so that the proximal elements 610 are in their raised position. Pushing the proximal element line handles 1402, 1404 in a distal direction moves the handles to their second position (see FIG. 14C) which releases the tension and correspondingly lowers the proximal elements 610. The base 1401 and the proximal element line handles 1402, 1404 may together form a ball-detent mechanism or a snap-fit mechanism, for example, which may releasably and respectively secure the proximal element line handles 1402, 1404 in the first and second position. As illustrated in FIG. 12B, the base 1401 is disposed within the housing 1212 such that, when the proximal element line handles 1402, 1404 are in an example of their second position, a majority of their respective lengths are disposed within the base 1401 and the housing 1212. This helps secure the gripper control assembly 1400 and provide the gripper control assembly 1400 a low profile.

A first sheath 1422 and a second sheath 1424 may extend from a distal end of the base 1401 and to a manifold 1470 of the fluid management assembly 1450, as described in more detail below. In some examples, the first proximal element line 804a may extend through the first sheath 1422, and the second proximal element line 804b may extend through the second sheath 1424. The sheaths 1422, 1424 may help shield the proximal element lines 804a, 804b from rubbing or snagging on other components within the handle 1210 as they are actuated. Additionally, the sheaths 1422 and 1424 help prevent fluid from escaping the fluid management assembly 1450 and or contaminants from entering the fluid management assembly 1450.

As shown in FIGS. 12B, 14A, and 15C, the fluid management assembly 1450 may generally include a fluid chamber 1460, a connector 1480, and a manifold 1490 separating the fluid chamber 1460 from the connector 1480 according to various examples of the disclosure. The fluid chamber 1460 may also be a lock handle housing, as described in more detail below. The fluid chamber 1460 may include a chamber volume 1468 and a chamber lid 1464 covering the chamber volume 1468 in some examples. In some examples, an O-ring 1463 seals the chamber lid 1464 to the fluid chamber 1460, as best shown in FIG. 15C. The chamber lid 1464 may include a first inlet/outlet port 1452a which may define a luer lock for connection to a flush bag or the like for delivering/receiving fluid to or removing air from the fluid chamber 1460 in some examples. The fluid chamber 1460 may also include a lock handle extension 1101 extending from a side thereof according to various examples. Such lock handle extension 1101 may be configured to receive a lock line knob, as described further below.

Additionally, the fluid chamber 1460 may include a first opening 1466 at proximal end and a second opening 1467 at a distal end, as shown in the example of FIG. 15C.

The connector 1480 may be disposed distal of the fluid chamber 1460 and may be configured to connect to a proximal end portion 1221 of the delivery catheter 850, such as over at least a portion of the proximal end portion 1221 of the delivery catheter 850, in some examples. In this regard, the proximal end portion 1221 of the delivery catheter 850 may be connected to the connector 1480 such that fluid may flow between the connector 1480 and the delivery catheter 850 lumens to lubricate the proximal element lines 804a, 804b, the lock line 808 and the actuator rod 810 and over an exterior of the delivery catheter 850 to ensure smooth translation of the delivery catheter 850 relative to a brake shaft 1230, in some examples. The connector 1480 may also include a second inlet/outlet port 1454 for fluid transfer, for example. The second port 1454, however, may extend from an opposite side of the handle 1210 from the first inlet/outlet port 1452a in various examples.

The manifold 1490 may be positioned between the fluid chamber 1460 and the connector 1480 and may be connected to a proximal end of the connector 1480, as shown in the example of FIGS. 12B and 14A. The manifold 1490 may include a shaft 1492 extending proximally therefrom which may be coupled to the second opening 1467 of the fluid chamber 1460 in some examples. The shaft 1492 may have a plurality of lumens extending therethrough each for receipt of any one of the actuator rod 810, a first end portion 808a of the lock line 808, and a second end portion 808b of the lock line 808 (see e.g., FIG. 15M). The second opening 1467 of the fluid chamber 1460 may include an O-ring 1467a and a ring retainer 1467b to provide a leak free seal between the fluid chamber 1460 and the shaft 1492, for example. As illustrated in the example of FIG. 14A, the actuator rod 810 may extend into the first opening 1466 of the fluid chamber 1460 and may pass through the fluid chamber 1460 into the shaft 1492 of the manifold 1490 where it may then be directed into the delivery catheter 850. The first opening 1466 of the fluid chamber 1460 may include a mandrel seal 1466a and a mandrel retainer 1466b (see FIG. 15C) for sealing the interface between the actuator rod 810 and the fluid chamber 1460 in various examples. The sheaths 1422, 1424 may be connected to the manifold 1490 such that the proximal element lines 804a, 804b extending through the sheaths 1422, 1424 are directed through the manifold 1490 and into the delivery catheter 850 according to various examples.

In some examples, fluid may enter the fluid management assembly 1450 via the second port 1454 and into the connector 1480. Fluid from the connector 1480 may then travel distally into the lumens of the delivery catheter 850 to lubricate the control elements therein, such as the proximal element lines 804a, 804b, and the lock line end portions 808a and 808b, for example. Additionally, fluid from the connector 1480 may flow distally between the delivery catheter 850 and the brake shaft 1230 to lubricate relative translation, as described in more detail below. Fluid may also travel proximally from the connector 1480 through the manifold 1490 and into the sheaths 1422, 1424 to lubricate the proximal element lines 804a, 804b and through the lock line lumens within the shaft 1492 and into the fluid chamber 1460. Once fluid is located within the fluid chamber 1460, it may then travel distally through a lumen for the actuator rod 810 which may pass through the shaft 1492 and into the delivery catheter 850. The first inlet/outlet port 1452a may be used to deair the system.

The arrangement of the fluid management assembly 1450, as described above and shown in the figures, significantly reduces the volume of fluid delivered to and from the fluid chamber 1460 as compared to prior delivery systems and provides a single point flush and anchoring to the handle 1210 while ensuring fluid runs within the delivery catheter 850 lumens and over an outer surface of the delivery catheter 850. Such reduction may be upwards of 3.7 times that of prior delivery systems and results in a more efficient deairing of system with a reduction in the amount of flush bags utilized.

Fluid volume of the fluid management assembly 1450 may be about 14 cubic centimeters, for example. Such reduction in volume is facilitated at least by the knob configuration of the lock control assembly 1500, which is described in more detail below. The fluid management assembly 1450 also decreases overall preparation time and decreases the risk that a flush line may become tangled at least due to a reduced number of flush line connections (e.g., one total flush line connection to the second port 1454).

Furthermore, the arrangement of the fluid management assembly 1450 reduces the number of hemostatic valves that may be needed which correspondingly increases the torque transmission ratio from the delivery catheter handle 1210 to the fixation device 500.

FIGS. 15A-150 depict the lock control assembly 1500 according to an implementation of the present disclosure. The lock control assembly 1500 may actuate the lock 1000 between its locked and unlocked configuration, for example. The lock control assembly 1500 may, for example, also be configured to release the lock line 808 from the fixation device 500. The lock control assembly 1500 may generally include one or more of a lock line handle 1502, the lock handle housing 1460, a spool 1560, and a locking system 1504.

Referring to the example of FIG. 15C, the lock line handle 1502 may generally include a lock knob 1510 and a lock handle shaft 1540 extending from the lock knob 1510.

In some examples, the lock knob 1510 may be configured to be gripped by a user and pivoted relative the delivery system handle 1210 to operate the lock line 808 (as described in greater detail below). The lock knob 1510 can have a pear shape which can act as a flag or pointer to help indicate when the lock knob 1510 is in a locked or unlocked position. Although shown as a pear shape, the lock knob 1510 can have any suitable shape and/or configuration. Also, the lock knob 1510 may have a recess 1513 which may also be pear shaped. However, other shapes are contemplated such as rectangular or oval, for example. Additionally, the lock knob 1510 may have an opening 1511 (e.g., a cylindrical shaped opening) extending therethrough and intersecting the recess 1513.

In the implementation depicted, the lock knob 1510 may include a lock knob insert 1520 and a lock knob cap 1512. The lock knob insert 1520 may include an insert body 1522 that may be cylindrical and a flange 1521 that may be connected to one end of the insert body 1522. The flange 1521 may have the same (i.e., corresponding) shape as the lock knob recess 1513 in various examples. Thus, where the lock knob recess 1513 is pear shaped, the flange 1521 may have a pear shape. The insert body 1522 may have an inner surface that may define an opening 1525 that may extend entirely through the insert body 1522 in some examples. Inner surface of the insert body 1522 may include a plurality of teeth or splines that may be configured to enmesh with corresponding teeth or splines 1554 on the lock handle shaft 1540 (see e.g., FIG. 15F), as discussed further below. The insert body 1522 may also include a channel 1524 extending circumferentially about an exterior of the insert body 1522 and may include a plurality of detents 1523 which may each extend radially inwardly and, in some examples, may intersect the channel 1524, for example. In various assembled examples, the flange 1521 may be disposed within the recess 1513 of the lock knob 1510 while the insert body 1522 may extend through the cylindrical opening 1511 of the lock knob 1510. The shape (e.g., pear) of the lock knob recess 1513 and corresponding shape (e.g., pear) of the flange 1521 can limit relative rotation between the lock knob insert 1520 and the lock knob 1510. The lock knob cap 1512 may be connected to the lock knob 1510, such as via a snap-fit connection and/or threaded connection, for example. The lock knob cap 1512 may be positioned over the flange 1521 of the lock knob insert 1520 which may sandwich the flange 1521 between the lock knob 1510 and the lock knob cap 1512 thereby retaining the lock knob insert 1520. The lock knob cap 1512 may also have an opening 1512a that may align with the opening 1525 of the lock knob insert 1520, as shown in the example of FIG. 15C, and may provide clearance for a lever arm 1542 of the lock handle shaft 1540 to be partially positioned therein in a locked position.

Although the lock knob 1510, the lock knob insert 1520, and the lock knob cap 1512 are shown and described as being separate components, each of these components can be integrated together so as to form a monolithic lock knob structure, such as by an injection molding process or additive manufacturing process, for example.

FIGS. 15D-15H depict some examples of the lock handle shaft 1540. The lock handle shaft 1540 may generally include one or more of a shaft body 1550 and the lever arm 1542 pivotably connected to the shaft body 1550. The shaft body 1550 may include a first end portion 1552 and a second end portion 1553. An opening 1551 may extend through the length of the shaft body 1550 from the first end portion 1552 to the second end portion 1553. As shown in the example of FIGS. 15E and 15F, the opening 1551 may be larger at the first end portion 1552 than at the second end portion 1553. The first end portion 1552 of the shaft body 1550 may be substantially cylindrical and may be configured to be received within a corresponding opening 1565 in the spool 1560 (see e.g., FIG. 15B). A notch 1556 may be formed in the first end portion 1552, and a fin or tab 1555 may extend radially outwardly from the first end portion 1552 in some examples. The fin 1555 may be configured to be received within a corresponding notch 1562 in the spool 1560 (see FIG. 15C), for example. In the implementation depicted, the fin 1555 and the notch 1562 may be circumferentially aligned. The shaft body 1550 may, for example, also include an O-ring 1559 positioned between the first and second end portions 1552, 1553 for forming a seal between the lock handle shaft 1540 and the lock handle extension 1101 of the lock handle housing 1460 when received therein.

Additionally, the shaft body 1550 may include the teeth 1554 which may enmesh with the teeth of the lock knob insert 1520 which may rotationally constrain the lock handle shaft 1540 relative to the lock knob insert 1520 when disposed therein according to various examples of the disclosure. As shown in the example of FIGS. 15F and 15G, the second end portion 1553 may include a post opening 1553a for a corresponding post 1544 of the lever arm 1542. Also, the second end portion 1553 may include an abutment feature 1557 extending radially outwardly therefrom which may be configured to constrain a range of motion of the lever arm 1542 when coupled to the shaft body 1550, for example.

In various examples, the lever arm 1542 of the lock handle shaft 1540 may include a lever body 1545 and the post 1544 extending from the lever body 1545. The post 1544 may be rotatably received within the post opening 1553a of the shaft body 1550 in some examples. The lever arm 1542 may also include a first opening 1541a that passes through the lever body 1545 and a second opening 1541b that passes through the post 1544 (see the example of FIG. 15H). However, in some implementations, the lever arm 1542 may only include the second opening 1541b passing through the post 1544.

The first and second openings 1541a, 1541b may be axially aligned in various examples, As shown in the example of FIG. 15N, the lock line 808 may include the first end portion 808a and the second end portion 808b. The first end portion 808a may be fixedly secured to the shaft body 1550, such as to an inner surface thereof. Prior to removal of the lock line 808 from the fixation device 500, the second end portion 808b of the lock line 808 may extend through the opening 1551 in the shaft body 1550, and the first and second openings 1541a, 1541b of the lever arm 1542. In some examples, the lever arm 1542 may be rotatable about an axis of the post 1544 and relative to the shaft body 1550 between a first position or unlocked configuration (see FIGS. 15D, 15E, and 15N) and a second position or locked configuration (see FIGS. 15F and 15G). In some examples of the unlocked configuration, the lever arm 1542 may be axially aligned with the shaft body 1550 such that the first and second openings 1541a, 1541b of the lever arm 1542 are coaxially aligned with the opening 1551 of the shaft body 1550. This may allow the second end portion 808b of the lock line 808 to be passed through the lock handle shaft 1540 as it is pulled away from the delivery system handle 1210, as best shown in FIG. 15N. In some examples of the locked configuration, the lever arm 1542 may be rotated such that it is angled relative to the shaft body 1550. In this regard, the second opening 1541b within the post 1544 may be angled relative to the opening 1551 in the shaft body 1550 such that a segment of the lock line 808 is trapped between the post 1544 and the shaft body 1550. For example, the lever body 1545 may be rotated 90 degrees relative to the shaft body 1550 so that the lock line 808 may be trapped and compressed along an arc subtending an angle of about 90 degrees and the second opening 1541b is oriented orthogonal to the opening 1551. However, in other implementations, the lever body 1545 may be rotated to angles less than or greater than 90 degrees, such as 45 degrees to 180 degrees, for example. This may secure the second end portion 808b of the lock line 808 to the lock handle shaft 1540 to prevent it from being decoupled from the fixation device 500 until desired. Although it has been described that the shaft body 1550 includes a female post opening and the lever arm 1542 includes a male post, it is also contemplated that the shaft body 1550 may include the male component, while the lever arm 1542 may include the female component. In other words, the shaft body 1550 may include a post, like that of the post 1544, with a lock line opening extending therethrough, while the lever arm 1542 may include a post opening, like that of the post opening 1553a, and a lock line opening, such as the first opening 1541a, intersecting such post hole. The lever arm 1542 may be provided with a readily identifiable color as compared to other features of the lock control assembly 1500 so as to identify the lever arm 1542 with fixation device deployment in some examples. Such color may be magenta, for example.

As illustrated in the example of FIGS. 15C and 15N, the lock handle shaft 1540 may extend through the lock knob insert 1520 and the lock knob 1510 so that the lever arm 1542 extends from the lock knob 1510 and the first end portion 1552 of the shaft body 1550 extends from the lock knob insert 1520 so that it can be received with the spool 1560. The teeth 1554 of the shaft body 1550 may mesh with the teeth of the lock knob insert 1520 so as to rotationally constrain the shaft body 1550 relative to the lock knob insert 1520 and allow them to rotate together in some examples. The lock handle shaft 1540 may be secured to the lock knob 1510 via a hairpin clip 1530 which may be inserted between the lock knob 1510 and the lock knob cap 1512 and engage a circumferential groove 1558 in the shaft body 1550 according to various examples. In this arrangement, the lever arm 1542 may be moved between the locked and unlocked configuration to correspondingly secure and release the second end portion 808b of the lock line 808. The abutment feature 1557 may limit rotation of the lever arm 1542 so that when the abutment feature 1557 stops the rotation of the lever arm 1542, it is in the unlocked configuration, according to various examples of the disclosure.

The spool 1560 may have the opening 1565 for receipt of the first end portion 1552 of the lock handle shaft 1540, as shown in the example of FIG. 15B. Additionally, the spool 1560 may include the notch 1562 which may be configured to receive the fin 1555 of the shaft body 1550 and a first lock line opening 1564a which may be aligned with the notch 1562 and may extend through a track 1566 of the spool 1560, as best shown in the example of FIG. 15C. This may allow the first end portion 808a of the lock line 808 to pass through the notch 1556 in the shaft body 1550 and through the first lock line opening 1564a so that it can be partially wrapped around the spool 1560 and extend through the second opening 1467 in the fluid chamber 1460 to the delivery catheter 850, as shown in FIG. 15B. In some examples, the spool 1560 may include a second lock line opening 1564b which may extend through the track 1566 of the spool 1560 and through a side of the spool 1560 which may allow the second end portion 808b of the lock line 808 returning from the delivery catheter 850 to extend through the second lock line opening 1564b out of the spool 1560 and into the opening 1551 of the lock handle shaft 1540, as shown in FIG. 15B.

As mentioned above, the lock handle housing 1460 may also be the fluid chamber of the fluid management assembly 1450. In some examples, the spool 1560 may be disposed within the chamber volume 1468 of the lock handle housing 1460 such that it is aligned with the lock handle extension 1101 extending therefrom, as shown in FIG. 15B. As mentioned above, the spool 1560 can be rotationally fixed relative the lock handle shaft 1540, for example, by a keying feature, such as the fin 1555 and the notch 1562. Accordingly, rotation of the lock knob 1510, which can cause rotation of the lock handle shaft 1540, can thereby cause rotation of the spool 1560 and therefore increase or decrease tensile load on the lock line 808 by way of the lock line 808 spooling or unspooling around the spool 1560. The lock handle housing 1460 can include a dowel pin 1469a in some examples. The first end portion 808a of the lock line 808 can be routed around the dowel pin 1469a and a fin 1469b in the lock handle housing 1460. This can align the first end portion 808a of the lock line 808 with the track 1566 of the spool 1560 and can manage slack in the first end portion 808a of the lock line 808. If slack is not managed, the first end portion 808a of the lock line 808 can disengage from the track 1566 and from the spool 1560. As noted above, the first end portion 808a and the second end portion 808b of the lock line 808 may extend through the second opening 1467 in the lock handle housing 1460 so as to extend to and from the delivery catheter 850, respectively. In some examples, the first end portion 808a can be routed around the dowel pin 1469a and the fin 1469b and routed at least partially around the spool 1560 along the track 1566 to the first lock line opening 1564a where it passes through the spool 1560 and enters the opening 1551 of the lock handle shaft 1540 via the notch 1556. The first end portion 808a may be fixedly secured to the shaft body 1550 in some examples.

Additionally, in some examples, the second end portion 808b may be routed around the dowel pin 1469a, through the second lock line opening 1564b in the spool 1560, and through the opening 1551 in the lock handle shaft 1540 where it may be releasably secured via the lever arm 1542.

The delivery system 1200 can be flushed, for example, via the first inlet/outlet port 1452a in the chamber lid 1464 of the lock handle housing 1460. For example, flush fluid can enter through the first inlet/outlet port 1452a into the lock handle housing 1460 and out through the second opening 1467 into the connector 1480. During preparation of the delivery system 1200, air can exit the delivery system handle 1210 through the second inlet/outlet port 1454 of the connector 1480 in some examples.

In some examples, the lock handle extension 1101 may rotatably receive the lock handle shaft 1540 of the lock line handle 1502 and the insert body 1522 of the lock knob insert 1520. As mentioned above, the lock control assembly 1500 may include the locking system 1504 to limit movement of the lock line handle 1502 relative the delivery system handle 1210 and removal of the lock line handle 1502 relative the delivery system handle 1210. The locking system 1504 can be a latch-detent lock in one illustrative example. For example, the latch can include a distal U-lock 1570 and a proximal U-lock 1580 coupled to one another (and collectively referred to as the “U-lock” or “lock body”) and configured to surround the lock handle extension 1101, as shown in FIGS. 15B and 15C. The proximal U-lock 1580 can include a pin 1582 which can engage the detents 1523 on the lock knob insert 1520, when the pin 1582 is insert through a window 1102 disposed in the lock handle extension 1101. In addition, as mentioned above, the channel 1524 may intersect the detents 1523. Thus, the pin 1582 may also engage the channel 1524 when the pin 1582 is not engaging the detents 1523. This helps prevent the lock knob insert 1520 and the rest of the lock line handle 1502 from being inadvertently removed when rotating the lock knob insert 1520 between the different positions (i.e., locked, unlocked, and third position described below). The distal U-lock 1570 can include a rail 1572 to engage the channel 1524 on the lock knob insert 1520 according to some examples.

In some examples, the locking system 1504 can further include a delivery handle insert 1590 and a release slider 1594, as shown in FIG. 15C, which can be used to release the lock knob 1510. The delivery handle insert 1590 can include an indication, for example, an icon (such as a lock) or writing, to indicate that the delivery handle insert 1590 and the release slider 1594 are intended to lock the lock line handle 1502. For example, and not by way of limitation, the release slider 1594 can include a pin 1596 that can engage the proximal U-lock 1580 (for example, through a window 1591 in the delivery handle insert 1590). A spring 1584 or other biasing mechanism can be supported on a pin 1104 of the lock handle extension 1101 and can bias the latch into the locked position, for example. That is, the spring 1584 can bias the latch in a distal direction to cause the pin 1582 to engage the detents 1523. Engagement of the pin 1582 with the detents 1523 can provide feedback, for example, audible and/or tactile, for a user that the lock knob 1510 has reached a specific position (e.g., lock, unlock, override (also referred to herein as the third position)). The pin 1582 and the detents 1523 can be arranged such that the lock knob 1510 will not move from a specific position (e.g., lock, unlock, override), however, a user can move the lock knob 1510 (i.e., overcome the engagement between the pin 1582 and the detents 1523) by turning the lock knob 1510, and without touching the release slider 1594. A portion of the distal U-lock 1570, for example, the rail 1572 on the distal U-lock 1570 and the channel 1524 can be arranged such that the lock line handle 1502 can be removed from the delivery catheter handle 1210 when the distal U-lock 1570 is disengaged from the channel 1524 of the lock knob insert 1520. For example, a user can slide the release slider 1594 to disengage the distal U-lock 1570 from the channel 1524 and thereby release the lock line handle 1502. For example, sliding the release slider 1594 in the proximal direction can disengage the rail 1572 on the distal U-lock 1570 from the channel 1524 and thereby permit the user to pull the lock line handle 1502 from the delivery catheter handle 1210.

In an exemplary operation, the fixation device 500 can be delivered in a locked position (as described above). The lock knob 1510 can be in the locked position, which is shown for example, in FIGS. 12B and 15I. In the locked position, the lock line 808 can have no (or relatively low) tension and the lock knob 1510 can point distally.

The spring 1584 can bias the distal U-lock 1570 and the proximal U-lock 1580 in a distal direction and therefore bias the pin 1582 to engage the detents 1523 of the lock knob insert 1520 (FIG. 15I).

During some examples of a procedure, the user can turn the lock knob 1510 to the unlocked position to apply tension (or relatively higher tension) to the lock line 808. Turning the lock knob 1510 can overcome the engagement of the pin 1582 with the detents 1523. For example, the lock knob 1510 can be rotated in a clockwise rotation (as viewed in FIG. 12A) such that the lock knob 1510 can point upwardly (FIG. 15J). In such a position, the lock knob 1510 itself can indicate to the user that the fixation device 500 is in the unlocked position. Rotating the lock knob 1510 can cause the spool 1560 to rotate (as described above) and can increase tension on the lock line 808 as the lock line 808 spools around the spool 1560. Such tensioning may tension both the first end portion 808a and the second end portion 808b of the lock line 808 so as to have an even pull on the lock 1000. As noted above, increasing tension on the lock line 808 can apply a force on the release harness 1010 which can unlock the fixation device 500, as described above. The spring 1584 can bias the distal U-lock 1570 and the proximal U-lock 1580 in a distal direction and therefore bias the pin 1582 to engage the detents 1523 of the lock knob insert 1520 (FIG. 15J) and bias the rail 1572 on the distal U-lock 1570 to engage the channel 1524 of the lock knob insert 1520. The detents 1523 (as well as labeling) can define the locked and unlocked positions. A click sound can be made as the pin 1582 engages the detents 1523. The detents 1523 can also prevent the lock knob 1510 from returning to the locked position (until a user engages the lock knob 1510).

In some examples, the user can lock the fixation device 500 by rotating the lock knob 1510 in the counterclockwise direction (as viewed in FIG. 12A) to return the lock knob 1510 to the locked position, unspool the lock line 808 from the spool 1560, and decrease tension on the lock line 808. A click sound can be made as the pin 1582 engages the detents 1523. The rail 1572 can remain engaged with the channel 1524 even as the lock line handle 1502 moves between the locked, unlocked, and third position according to an example of the disclosure. In some examples, the lock knob 1510 can be rotated beyond the unlock position (see e.g., FIG. 15K), to a third position, and to thereby further increase tension on the lock line 808. This can be useful during certain procedures to ensure that the fixation device 500 properly locks. In various examples, additional detents 1523 can be provided to engage the pin 1582 and hold the lock knob 1510 in the third position or further rotated positions. The rail 1572 can remain engaged with the channel 1524 even as the lock knob 1510 moves between the locked, unlocked, and third positions. Although described in a particular arrangement, the third position, and any further positions, can include any suitable arrangement.

Before the fixation device 500 can be deployed from the delivery system 1200, the lock line 808 may be decoupled from the fixation device 500. To remove the lock line 808, in some examples, the user can ensure that the lock knob 1510 is in the locked position. The user can also rotate the lever arm 1542 of the lock handle shaft 1540 to the unlocked position and retract the release slider 1594 (i.e., the release slider 1594 can be pulled in the proximal direction). This can pull the proximal U-lock 1580 (for example via the pin 1596) and the distal U-lock 1570 in a proximal direction (FIGS. 15L and 15M), which can disengage the rail 1572 of the distal U-lock 1570 from the channel 1524. The user can pull the lock line handle 1502 off the delivery catheter handle 1210 (FIG. 15N). Withdrawing the lock line handle 1502 while the lever arm 1542 is in the unlocked position can release the second end portion 808b of the lock line 808 from within the lock handle shaft 1540, while the first end portion 808a remains fixed to the lock handle shaft 1540. Accordingly, in this example, withdrawing the lock line handle 1502 pulls one end portion of the lock line 808 (i.e., the first end portion 808a), while the other end portion (i.e., the second end portion 808b) is free to travel distally along the length of the lock handle shaft 1540, through the fixation device 500, and proximally along the length of the delivery catheter 850. Therefore, pulling the lock line handle 1502 away from the delivery catheter handle 1210 withdraws the lock line 808 from the delivery system 1200 and the fixation device 500.

If the first end portion 808a of the lock line 808 becomes stuck, the user can disassemble the lock line handle 1502 (FIG. 150). For example, the user can remove the hairpin clip 1530 and remove the lock knob 1510 and the lock knob insert 1520 from the lock handle shaft 1540. This can provide direct access to the first end portion 808a which is attached to the lock handle shaft 1540. The user can pull the second end portion 808b of the lock line 808 to pull to free the “stuck” first end portion 808a. As described, the lock control assembly 1500 is configured to provide discrete binary lock and unlock positions with tactile feedback which helps the clinician know when the locked and unlocked conditions are achieved. Additionally, the lock control assembly 1500 with the lock knob 1510 controls and limits the forces exerted on the fixation device 500 thereby protecting its components from aggressive end user use and high lock line tension loads. Other exemplary lock control assemblies that may be implemented in the interventional system 1100 are disclosed in U.S. Pat. No. 11,622,859, the disclosure of which is hereby incorporated by reference herein in its entirety.

The delivery catheter 850 may include a proximal end portion 1221 and a distal end portion 1222, as best shown in FIG. 12B. As mentioned above the proximal end portion 1221 of the delivery catheter 850 may be connected to the delivery catheter handle 1210 and, in particular, may be coupled to the connector 1480 of the fluid management assembly 1450. The delivery catheter 850 may include a plurality of lumens 852 extending from the proximal end portion 1221 to the distal end portion 1222. Each lumen may receive one of the first proximal element line 804a, the second proximal element line 804b, the actuator rod 810, and the first end portion 808a and the second end portion 808b which each extend through the distal end portion 1222 of the delivery catheter 850 and to the fixation device 500 where they are respectively coupled to the proximal elements 610, the stud 540 or the base 532, and the lock 1000. The distal end portion 1222 of the delivery catheter 850 may include the shaft 810 extending distally therefrom for coupling to the fixation device 500. As discussed above, the actuator rod 810 may extend through the shaft 810 and into the fixation device 500 where it may be coupled to the stud 540 or the base 532. The distal end portion 1222 of the delivery catheter 850 may optionally include a nose 854. In the implementation depicted, the nose 854 has a flanged shape. Such a flanged shape prevents the nose 854 from being retracted into a guide catheter of the steerable guide system 1105. However, the nose 854 may have any shape including bullet, rounded, blunt or pointed, to name a few.

During a transcatheter procedure it may be desirable to advance and/or retract the delivery catheter 850 relative to the steerable guide system 1105 and to secure the delivery catheter 850 in a desired position relative to the target valve. FIGS. 12B and 16A-17B depict a delivery catheter fastening assembly 1600 according to an implementation of the present disclosure and which may be configured to selectively secure the delivery catheter 850 from translation and release the delivery catheter 850 for proximal-distal translation. In the implementation depicted, the delivery catheter fastening assembly 1600 may generally include the delivery catheter 850, a brake shaft 1230, a distal end portion 1610 of the delivery catheter handle 1210, a fastener knob 1620, and a compression ring 1530.

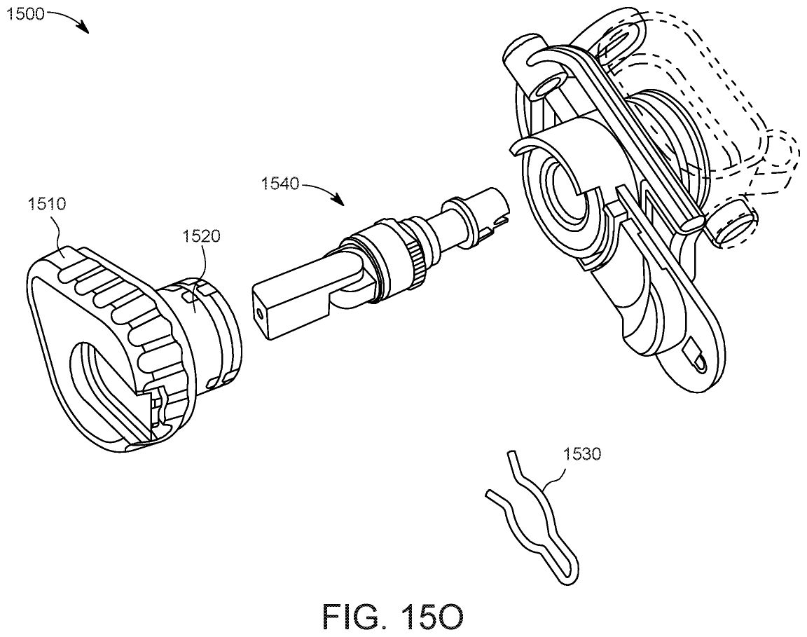

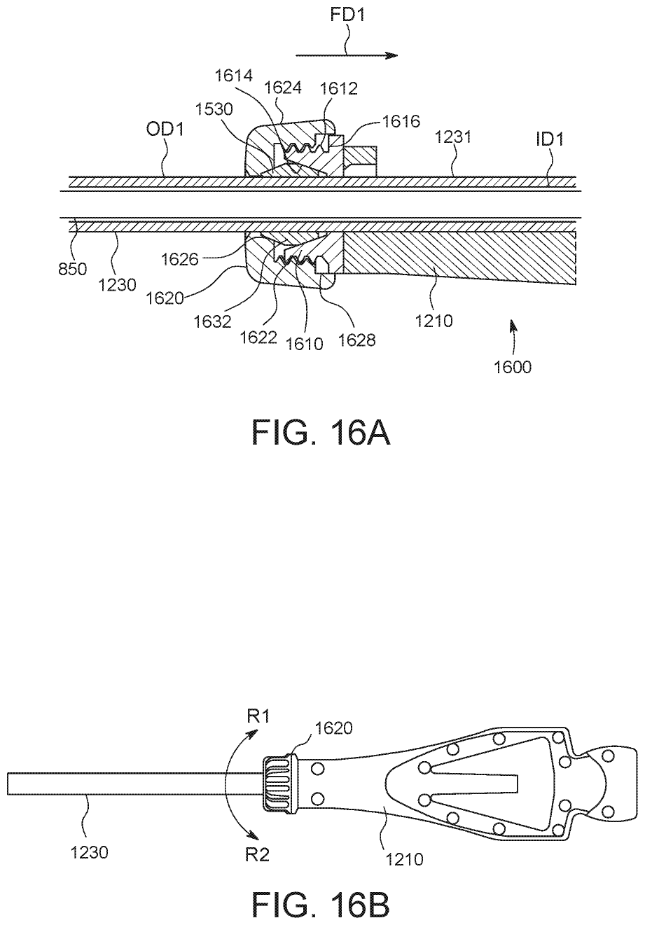

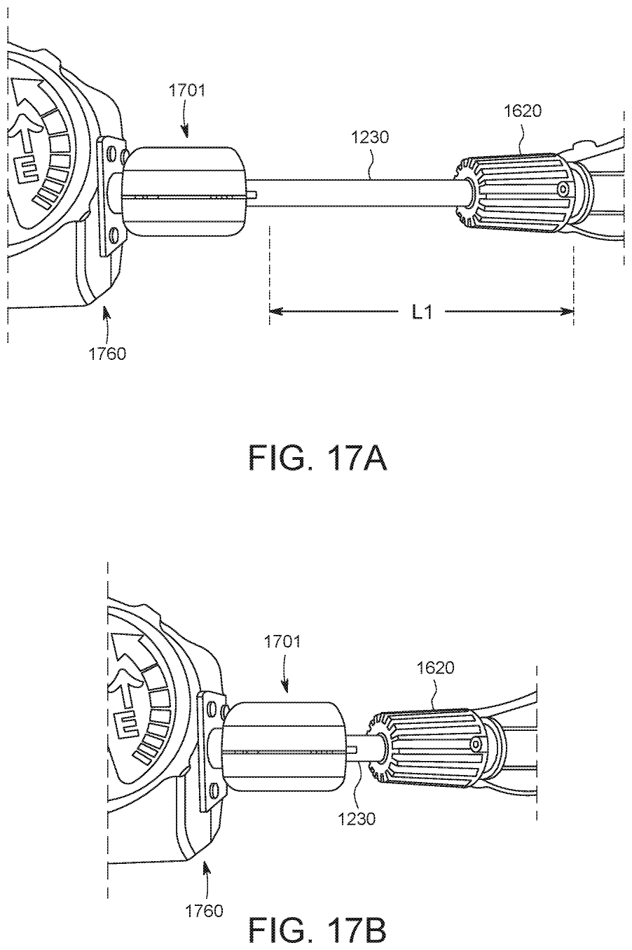

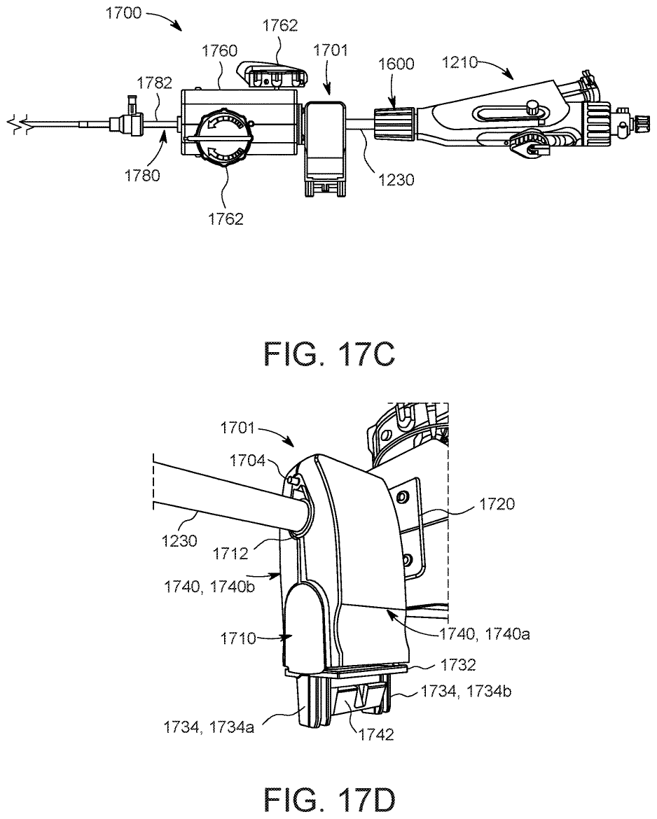

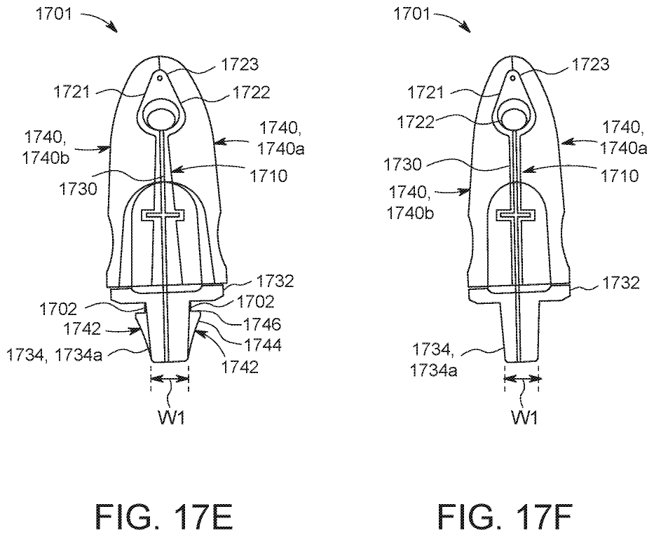

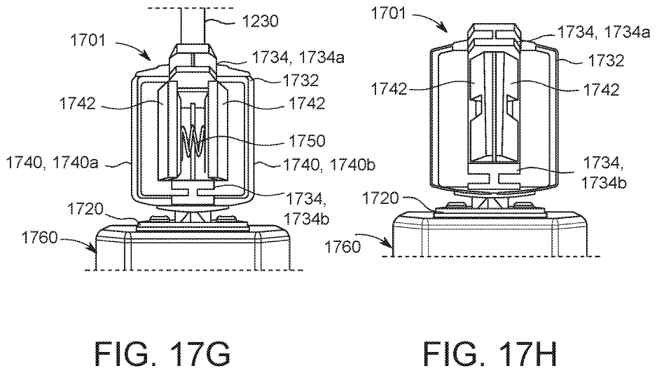

The brake shaft 1230 may, in some examples, have a first end portion 1231 that may extend through a distal opening of the delivery catheter handle 1210 and may be slidable relative thereto, as best shown in FIGS. 12B and 16A. The brake shaft 1230 may also include a second end portion 6032 that may be fixedly secured to an inner guide catheter handle 1760 of the steerable guide system 1105, as shown in FIGS. 17A and 17B and as described in more detail below. The brake shaft 1230 may include an outer diameter OD1 and an inner diameter ID1. The delivery catheter 850 may slidably and rotatably extend through the brake shaft 1230 and the inner diameter ID1 thereof, as shown in FIGS. 12B and 16A.

The distal end portion 1610 may be disposed at a distal end of the delivery catheter handle 1210 and may define the distal opening through which the brake shaft 1230 and the delivery catheter 850 extend. In this regard, the first end portion 1231 of the brake shaft 1230 may extend through the distal end portion 1610 such that the distal end portion 1610 circumferentially extends about the outer diameter OD1, as shown in FIG. 16A. The distal end portion 1610 may include external threads 1612 and an inner engagement section 1614. The inner engagement section 1614 may be an inner sloping surface or chamfered portion that may taper radially inwardly toward the brake shaft 1230 in a distal to proximal direction. The distal end portion 1610 may also have a stop surface 1616 disposed proximal of the external threads 1612.

The fastener knob 1620 may be a threaded ring that may circumferentially extend about the first end portion 1231 of the brake shaft 1230 and may include internal threads 1622 that may be configured to cooperate or matingly engage with the external threads 1612 of the distal end portion 1610, for example. The fastener knob 1620 may include an inner engagement section 1626 that may be an inner sloping surface or chamfered portion that tapers radially outwardly away from the brake shaft 1230 in a distal to proximal direction. An outer surface 1624 of the fastener knob 1620 may be tapered and/or may include textured or frictional features that may enhance grip.

Additionally, the fastener knob 1620 may have a stop-forming surface 1628 disposed proximal of the internal threads 1622 and which may correspond to the stop surface 1616 of the distal end portion 1610 which, when engaged, prevent further translation of the fastener knob 1620 in a fastening direction FD1. This has the benefit of providing an indication to a practitioner that the fastener knob 1620 has been adequately engaged with the distal end portion 1610 so as to reliably arrest translation of the brake shaft 1230 and the delivery catheter 850.

The compression ring 1530 may circumferentially extend about the outer diameter OD1 of the brake shaft 1230 such that the compression ring 1530 may come into direct contact with the outer diameter OD1, as shown in the example of FIG. 16A.

The compression ring 1530 may also be disposed between the respective inner engagement section 1614 and the inner engagement section 1626 of the distal end portion 1610 and the fastener knob 1620. The compression ring 1530 may include any suitable material including those with increased frictional properties. Such materials may include but are not limited to elastomeric or polymeric materials including medical-grade polymers, metal materials, ceramic materials, combinations thereof, or otherwise. The compression ring 1530 may define an outwardly bulging profile 1632 such that the outwardly bulging profile 1632 generally peaks at a mid-section of the compression ring 1530 and tapers inwardly towards its respective proximal and distal ends, according to some examples. In this regard, with the compression ring 1530 positioned between the inner engagement section 1614 and the inner engagement section 1626 of the distal end portion 1610 and the fastener knob 1620, the inner sloping surfaces of the distal end portion 1610 and the fastener knob 1620 correspondingly engage the outwardly bulging profile 1632, as illustrated in FIG. 16A.

During a transcatheter procedure it may be desirable to advance or retract the delivery catheter 850 to position the fixation device 500 within a target valve. In some examples, this may be achieved by unlocking the delivery catheter fastening assembly 1600 by rotating the fastener knob 1620 in a first rotation direction R1, as shown in FIG. 16B. Rotating the fastener knob 1620 in the first rotation direction RI advances the fastener knob 1620 in a distal direction which relieves compression on the compression ring 1530 and correspondingly friction on the brake shaft 1230. When the friction is sufficiently reduced, the delivery system handle 1210 may be translated in a proximal or distal direction along the brake shaft 1230 which correspondingly advances the delivery catheter 850 proximally or distally through the brake shaft 1230, as illustrated in FIGS. 17A and 17B. As the delivery system handle 1210 is advanced distally, the first end portion 1231 of the brake shaft 1230 is moved further proximally within the delivery system handle 1210. Conversely, as the delivery system handle 1210 is moved proximally, the first end portion 1231 of the brake shaft 1230 moves toward the distal end portion 1610 of the delivery catheter fastening assembly 1600. As shown in FIG. 17A, the maximum travel length of the delivery system handle 1210 relative to the brake shaft 1230 is a length L1. This length L1 may be limited so as to prevent the fixation device 500 from advancing too far and potentially damaging the heart H. For example, in a transfemoral approach to the mitral valve MV or the tricuspid valve TV, the fixation device 500 may be initially positioned within an atrium and advanced toward the valve from the atrium. The length L1 may be limited so as to not overshoot the valve and damage the chordae CT or ventricle. The length L1 may be about 3 to 4 inches, for example.

Once the fixation device 500 is in a desired position relative to the target valve, the delivery catheter fastening assembly 1600 may be locked by rotating the fastener knob 1620 in a second rotation direction R2 opposite the first rotation direction R1, as shown in the example of FIG. 16B. As the fastener knob 1620 is rotated about the distal end portion 1610 of the delivery system handle 1210, the fastener knob 1620 may translate in the longitudinal fastening direction FD1, which may be the proximal direction. As the fastener knob 1620 is translated in the fastening direction FD1, the inner engagement section 1614 and the inner engagement section 1626 of the distal end portion 1610 and the fastener knob 1620 compress the outwardly bulging profile 1632 of the compression ring 1530 which correspondingly compresses the compression ring 1530 against the outer diameter OD1 of the brake shaft 1230 until the friction resulting from such compression is sufficient to arrest proximal-distal translation of the delivery system handle 1210 relative to the brake shaft 1230. Stop-forming features 1616, 1628 of the distal end portion 1610 and the fastener knob 1620 may, for example, provide an indication that arrest has been achieved. By providing the integrated delivery catheter fastening assembly 1600, superfluous floating components, which in practice are easily and frequently lost, are minimized, as the fastener knob 1620 in cooperation with the distal end portion 1610 of the delivery system handle 1210 provide a simplified, effective, and intuitive system for restricting or arresting translation of the delivery catheter 850 relative to the brake shaft 1230 and correspondingly the steerable guide system 1105. Additionally, the fastener knob 1620 improves ergonomics and ease of repeated and precise use by the practitioner by providing a conveniently sized and located component for effectively arresting translation. Other delivery catheter fastener examples that may be implemented in the delivery system 1200 are disclosed in the '419 Publication mentioned above and are incorporated herein by reference.

FIGS. 11 and 17C-18C depict the steerable guide system 1105 according to an implementation of the present disclosure. The steerable guide system 1105 may be configured to guide the delivery catheter 850 and the fixation device 500 coupled thereto percutaneously through the cardiovascular system to a position near the target valve. In the implementation depicted, the steerable guide system 1105 generally includes an inner guide catheter assembly 1700 and an outer guide catheter assembly 1800. The inner guide catheter assembly 1700 may generally include one or more of the inner guide catheter handle 1760 and an inner guide catheter 1780 extending distally therefrom, and the outer guide catheter assembly 1800 may generally include one or more of an outer guide catheter handle 1810 and an outer guide catheter 1870 extending distally therefrom.

The outer guide catheter 1870 may have a proximal end 1872, a distal end 1874, and a central lumen extending therethrough, and the inner guide catheter 1780 may have a proximal end 1782, a distal end 1784, and a central lumen extending therethrough. The inner guide catheter 1780 may be positioned coaxially within the central lumen of the outer guide catheter 1870, as shown in FIG. 11. The distal ends 1784, 1874 of the catheters 1780, 1870, respectively, may be sized to be passable to a body cavity, typically through a body lumen such as a vascular lumen. The outer guide catheter 1870 and/or the inner guide catheter 1780 may be precurved and/or have steering mechanisms to position the distal ends 1784, 1874 in desired directions.

Steering of the outer guide catheter 1870 and the inner guide catheter 1780 may be achieved by actuation of one or more steering mechanisms. Actuation of the steering mechanisms may be achieved with the use of actuators which may be located on the inner guide catheter handle 1760 and the outer guide catheter handle 1810. The outer guide catheter handle 1810 may be connected to the proximal end 1872 of the outer guide catheter 1870 and may remain outside of a patient's body during use. The outer guide catheter handle 1810 may include one or more steering actuators or steering knobs 1812 which may be used to bend, arc, or reshape the outer guide catheter 1870, such as to form a primary curve. The inner guide catheter handle 1760 may be connected to the proximal end 1782 of the inner guide catheter 1780 and may optionally join with the outer guide catheter handle 1810 to form one larger handle. The inner guide catheter handle 1760 may include one or more steering actuator 1762 which may be used to bend, arc, or reshape the inner guide catheter 1780, such as to form a secondary curve and move or sweep the distal end 1784 of the inner guide catheter 1780 through one or more angles of motion.

The steerable guide system 1105 may have different configurations depending on the approach taken to the target valve and may differ depending on which valve is targeted. For example, approaching the tricuspid valve TV through the inferior vena cava IVC may involve tighter turns than approaching the mitral valve MV from the inferior vena cava IVC and across the interatrial septum S. Thus, the steering mechanisms and actuators 1762, 1812 of those steering mechanisms for the inner guide catheter assembly 1700 and the outer guide catheter assembly 1800 may be configured accordingly.

Examples of inner and outer guide catheter assemblies, steering mechanisms, and actuators thereof which may be incorporated into the interventional system 1100 and utilized to target the mitral valve MV are disclosed in U.S. Pat. No. 7,226,467, the disclosure of which is hereby incorporated by reference herein in its entirety. Also, examples of inner and outer guide catheter assemblies, steering mechanisms, and actuators thereof that may be incorporated into the interventional system 1100 and utilized to target the tricuspid valve TV are disclosed in U.S. Pub. No. 2023/0131595, the disclosure of which is hereby incorporated by reference herein in its entirety.

During a transcatheter procedure, it may be desirable to support and stabilize the steerable guide system 1105 and the delivery system 1200. However, it may also be desirable to move certain components thereof in a controlled manner. In this regard, the interventional system 1100 may include, in some examples, one or more of a proximal attachment assembly or first attachment assembly 1701 and a distal attachment assembly or second attachment assembly 1820.

FIGS. 17D-17H depict some examples of the proximal attachment assembly 1701. The proximal attachment assembly 1701 may be positioned between the inner guide catheter handle 1760 and the delivery system handle 6010. The proximal attachment assembly 1701 generally includes one or more of a support frame 1710 and a first engagement member 1740a and a second engagement member 1740b moveably attached to the support frame 1710.

In the implementation depicted, the support frame or support body 1710 may include an upper portion and a lower portion extending from the upper portion. The upper portion may include a mount member 1720 which may be configured to mount or otherwise connect to the inner guide catheter handle 1760, as shown in the example of FIGS. 17G and 17H. In the implementation depicted, the mount member 1720 may be in the form of a mounting plate which may be secured to the inner guide catheter handle 1760 via one or more fasteners. However, in other implementations, the mount member 1720 may be connected to the inner guide catheter handle 1760 via welding, for example, or the mount member 1720 may be integral with the inner guide catheter handle 1760 such as via an injection molding process, as another example.

The upper portion may, for example, also include a boss 1721 extending proximally from the mount member 1720. The boss 1721 may define a first opening 1722 and a second opening 1723 which may each extend at least partially into the boss 1721 and in a proximal-distal direction. The first opening 1722 may be configured to receive the brake shaft 1230 such that the brake shaft 1230 may pass through the proximal attachment assembly 1701 as it extends from the delivery system handle 6010 to the inner guide catheter handle 1760 where it is fixedly secured. However, in some implementations, the proximal attachment assembly 1701 may fixedly secure the brake shaft 1230 to the inner guide catheter handle 1760. The second opening or pin opening 1723 may be offset in an upward direction from the first opening 1722 and may be generally parallel relative thereto. The second opening 1723 may be configured to receive a hinge pin 1704, as shown in the example of FIG. 17D.

The lower portion of the proximal attachment assembly 1701 may include a central support wall or central member 1730, a transverse plate or transverse member 1732, and a first finger 1734a and a second finger 1734b. As shown in the example of FIGS. 17E and 17F, the central support wall 1730 may extend downwardly from the boss 1721 and may extend in a proximal-distal direction. The transverse plate 1732 may extend transverse to the central support wall 1730, such as perpendicular (+/−5 degrees) relative thereto, and may be connected to a lower end of the central support wall 1730 opposite an upper end thereof which may be connected to the boss 1721, according to some examples of the disclosure. The first finger or first end wall 1734a and the second finger or second end wall 1734b may extend from the transverse plate 1732 in a downward direction and may be respectively disposed at proximal and distal ends of the transverse plate 1732 so as to form a gap therebetween, for example. As shown in FIGS. 17E and 17F, the fingers 1734a, 1734b each define a width W1 which extends in a direction transverse to the proximal-distal direction.

The first engagement member 1740a and the second engagement member 1740b may be pivotably connected to the support frame 1710 in some examples. In this regard, the first engagement member 1740a and the second engagement member 1740b may, for example, be connected to each other via the hinge pin 1704 which extends through the boss 1721 of the proximal attachment assembly 1701, as mentioned above.

Such pivotable connection may be made at or near respective first ends of the engagement members 1740a, 1740b. As shown in the illustrated example, the first engagement member 1740a and the second engagement member 1740b extend about the boss 1721 and downwardly at opposite sides of the central support wall 1730. The boss 1721 may have a tear-drop shaped profile which may correspond to a tear-drop shape opening defined by the first engagement member 1740a and the second engagement member 1740b. This shape may facilitate pivotable action of the first engagement member 1740a and the second engagement member 1740b about the boss 1721. A spring 1750 may be positioned between the first engagement member 1740a and the second engagement member 1740b such that the spring 1750 biases the engagement members 1740a, 1740b toward a first position or locked configuration away from each other, as shown in FIG. 17G. In other words, when the first engagement member 1740a and the second engagement member 1740b are in the first position, they may be in an expanded state. However, pressing on the engagement members 1740a, 1740b against the bias of the spring 1750 may move the engagement members 1740a, 1740b to a second position or unlocked configuration in which they may abut each other, as shown in the example of FIG. 17H. Thus, when the first engagement member 1740a and the second engagement member 1740b are in the second position, they may be in a collapsed state according to some examples of the disclosure.

Each engagement member 1740a, 1740b may, in various examples, also include a hook member or capture member 1742 extending downwardly from a second end thereof. Such capture members 1742 may each extend through the transverse plate 1732 of the support frame 1710 and within the gap between the fingers 1734a, 1734b of the support frame 1710. Thus, in some examples of the first position, the first engagement member 1740a and the second engagement member 1740b may engage the transverse plate 1732 to limit or stop their outward expansion. The capture members 1742 may each include a sloping surface 1744 that slopes outwardly and terminates at a shoulder 1746 which faces the transverse plate 1732, as best shown in the example of FIG. 17E. Each shoulder 1746 and the transverse plate 1732 may define a slot or channel 1702 when the first engagement member 1740a and the second engagement member 1740b are in the first position. Such slot 1702 may be configured to slidably receive a corresponding rail of the stabilizer 1900, which is described in more detail below. Thus, in the example first position, the capture members 1742 may extend beyond the width W1 of the fingers 1734a, 1734b. In other words, the capture members 1742 in the first position can, in some examples, collectively define a width greater than the width W1 of the fingers 1734a, 1734b. However, according to some examples, when the engagement members 1740a, 1740b are in the second position, the capture members 1742 are either flush with or narrower than the width W1 of the fingers 1734a, 1734b. Although it has been described that the first engagement member 1740a and the second engagement member 1740b are each moveable relative to the support frame 1710, in some implementations one of such engagement members 1740a, 1740b may be static, while the other engagement member 1740a, 1740b may be dynamic. In other words, one of the engagement members 1740a, 1740b may only be moveable relative to the support frame 1710 between the first and second configurations (i.e., locked and unlocked configurations).

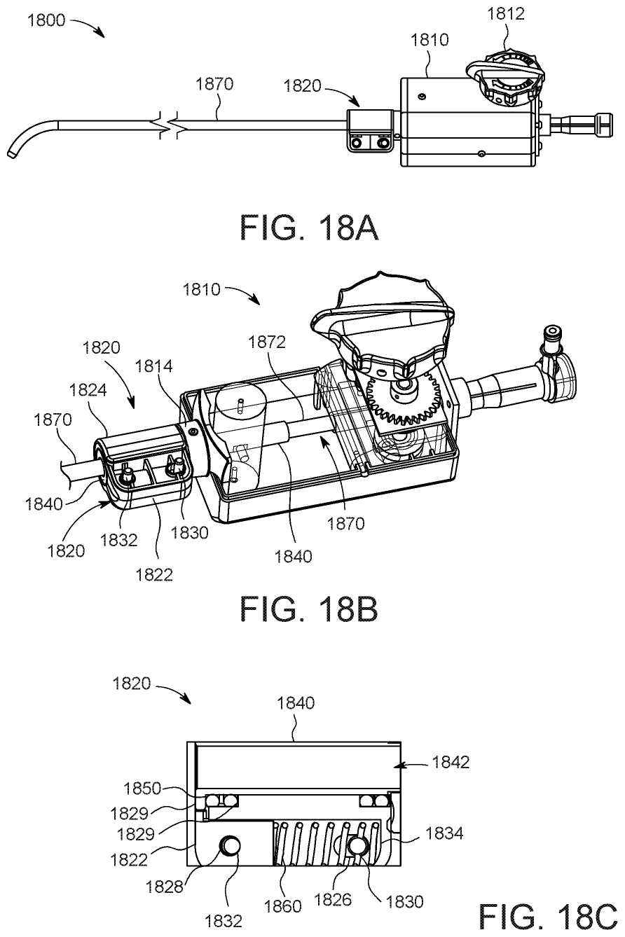

FIGS. 18B and 18C depict some examples of the distal attachment assembly 1820. The distal attachment assembly 1820 can be positioned distal of the outer guide catheter handle 1810. Thus, in the implementation depicted in FIG. 11, the inner guide catheter handle 1760 and the outer guide catheter handle 1810 are positioned between the proximal attachment assembly 1701 and the distal attachment assembly 1820. The distal attachment assembly 1820 may also have a snap-fit mechanism or the like. However, the snap-fit mechanism of the distal attachment assembly 1820 may be configured to constrain all movement when attached to the stabilizer 1900. The distal attachment assembly 1820 may generally include one or more of a bushing 1840, a housing 1819, and a connection mechanism for connecting to the stabilizer 1900.

The bushing or shaft 1840 may define a central opening 1842 through which the outer guide catheter 1870 may extend, as shown in the example of FIG. 18B. The bushing 1840 may extend into the outer guide catheter handle 1810 and be connected to a distal end of the outer guide catheter handle 1810 via a collar 1814 which may have a set screw that rotationally and translationally secures the bushing 1840 according to various examples.

The housing or support body 1820 may, in some examples, surround the portion of the bushing 1840 extending from the outer guide catheter handle 1810 and may include a lower housing portion 1822 and an upper housing portion 1824. As shown in the example of FIG. 18C, one or more O-rings 1850 may be positioned between the bushing 1840 and the housing 3220. For example, the distal attachment assembly 1820 may have four O-rings 1850 with two O-rings 1850 located at a proximal location and two O-rings 1850 located at a distal position. This distribution of the O-rings 1850 helps to ensure even distribution of load/compression. However, other configurations are contemplated, such as two or more O-rings 1850 distributed at even intervals along a length of the bushing 1840. To help contain the O-rings 1850, the lower housing portion 1822 and the bushing 1840 may have corresponding lips 1829, 1829 preventing proximal-distal travel of the O-rings 1850. The O-rings 1850 may be made from a material that enhances friction, such as silicon, for example. This arrangement helps provide rotational friction between the housing 3220 and the bushing 1840 and prevent unintentional rotation of the inner guide catheter 1780 and the outer guide catheter 1870 while utilizing the inner guide catheter handle 1760 and the outer guide catheter handle 1810. In other words, the O-rings 1850 help provide constant friction to prevent unintentional rotation of the inner guide catheter 1780 and the outer guide catheter 1870. This O-ring configuration also helps coaxially correct the inner guide catheter 1780 and the outer guide catheter 1870 to help keep them coaxially aligned. Furthermore, the O-rings 1850 help protect the outer guide catheter 1870 by removing direct contact of the outer guide catheter 1870 and by promoting handle torque transmission directly to the O-rings 1850 rather than directly from the handle 1810 to the catheter 1870 in order to brake the system.

The lower housing portion 1822 may include a connection mechanism for connecting the distal attachment assembly 1820 to the stabilizer 1900, for example. In the depicted implementation, the connection mechanism may include a dynamic pin or first engagement member 1830 and a static pin or second engagement member 1832, for example. The static pin 1832 may extend through a first opening 1828 in the lower housing portion 1822, while the dynamic pin 1830 may extend through a second opening 1826 in the lower housing portion 1822. In some examples, the second opening 1826 may be an elongate opening or elongate slot allowing the dynamic pin 1830 to dynamically move in a proximal-distal direction. The lower housing portion 1822 may include a chamber 1834 with a spring or other biasing element 1860 disposed therein, for example. The spring 1860 may bias the dynamic pin 1830 in a first position or proximal position.

When the spring bias is overcome, the dynamic pin 1830 may move to a second position or second position which is closer to the static pin 1832 than the first position. Thus, the dynamic pin 1830 and the static pin 1832 have a first configuration when the dynamic pin 1830 is in the first position and a second configuration when the dynamic pin 1830 is in the second position, according to an example of the disclosure.

FIGS. 19A-19C depict the stabilizer 1900 according to an implementation of the present disclosure. The stabilizer 1900 may generally include one or more of a base 1910, one or more support arms 1920, a distal attachment 1930, and a proximal attachment 1950.

The base or platform 1910 may be a plate with a flat bottom for placement on a flat surface of a table or the like. The base 1910 may define a longitudinal axis A3 and may include one or more handles 1912 for manipulating the stabilizer 1900. For example, as shown in FIG. 19A, the base 1910 may include one or more handles 1912 at proximal and distal ends of the base 1910.

The support arm 1920 may extend upwardly from the base 1910 and may support both the distal attachment 1930 and the proximal attachment 1950, as shown in the example of FIG. 19A. However, in other implementations, separate support arms 1920 may extend from the base 1910 that may separately support the distal attachment 1930 and the proximal attachment 1950.

The proximal attachment or first attachment 1950 may include one or more of a first rail or first member 1952, a second rail or second member 1954, a first end wall 1958, and a second end wall 1960. The first rail 1952 and the second rail 1954 may extend parallel (+/−5 degrees) to each other between the first end wall 1958 and the second end wall 1960 and may each have a planar upper surface 1956 and planar lower surface (not shown) according to some examples of the disclosure. The end walls 1958, 1960 and the rails 1952, 1954 may define an elongate slot 1962 that may be rectangular in shape. However, the elongate slot 1962 may also have other shapes, such as oval or pill-shaped, for example. The elongate slot 1962 may have a width slightly wider than the width W1 of the fingers 1734a, 1734b of the proximal attachment assembly 1701.

Additionally, the elongate slot 1962 may have a length L2 which may be defined from the first end wall 1958 to the second end wall 1960. As explained in more detail below, this length L2 may correspond to a distance that the inner guide catheter 1780 may translate relative to the outer guide catheter 1870. In this regard, the first end wall 1958 and the second end wall 1960 form stop surfaces for the proximal attachment assembly 1701 and correspondingly a translational limit for the inner guide catheter handle 1760.

The proximal attachment 1950 may also include proximal and distal tabs 1964, 1968 extending downward therefrom, for example. Such tabs 1964, 1968 may be used as leverage points for manual manipulation and translation of the inner guide catheter handle 1760 when the proximal attachment assembly 1701 is connected to the proximal attachment 1950. In some examples, the proximal attachment 1950 extends along a longitudinal axis A4 which may form an acute angle relative to the longitudinal axis A3 of the base 1910.

The distal attachment or second attachment 1930 may include a pair of attachment members 4032 and a recess 1931 extending therebetween. As shown in the example of FIG. 19B, each attachment member 4032 may include a first hook or first capture member 1936 and a second hook or second capture member 1938. The first capture member 1936 extends in a distal direction while the second capture member 1938 extends in a proximal direction such that each capture member 1936, 1938 defines a corresponding recess 1940, 1942 for receipt of the dynamic pin 1830 and the static pin 1832, respectively. Additionally, the first capture member 1936 may include a cam surface 1448 that slopes in a proximal to distal direction. Such cam surface 1448 may be configured to deflect the dynamic pin 1830 of the distal attachment assembly 1820 from its first position to its second position thereby allowing the dynamic pin 1830 to pass by the first hook 1936 and into its corresponding recess 1940 where it returns to the first position or somewhere between the first and second positions. Each attachment member 1932 may also include an intermediate projection 1444 that defines sloped surface 1446 that is sloped toward the second hook 1938 in various examples. This sloped surface 1446 may direct the static pin 1832 of the distal attachment assembly 1820 into the recess 1942. The attachment members 1932 may be laterally offset from each other and may extend parallel relative to each other (+/−5 degrees) so as to define the recess 1931 which may be configured to receive the lower housing portion 1822 of the distal attachment assembly 1820 according to various examples of the disclosure.

The stabilizer 1900 may be made from a single sheet of metal material such that at least the base 1910, the support arm 1920, and the proximal attachment 1950 are stamped from the single sheet of metal material and bent into their depicted configuration according to some examples of the disclosure. The distal attachment 1930 may be separately manufactured and connected to the support arm 1920, such as by welding, for example. However, in other implementations the distal attachment 1930 may also be stamped out of the single sheet of metal material. The depicted configuration of the stabilizer 1900 may reduce welding points, which may be potential points of failure, and may reduce cleaning and sterilization risks.

FIGS. 19D and 19E illustrate some examples of the connection between the distal attachment 1930 of the stabilizer 1900 and the distal attachment assembly 1820 of the steerable guide system 1105. In this regard, the distal attachment assembly 1820 may be attached to the distal attachment 1930 first before the proximal attachment assembly 1701. This may be achieved by sliding the static pin 1832 along the sloped surface 1446 of the intermediate projection 1444 and in the distal direction until the static pin 1832 is fully received within the recess 1942 and captured by the first hook 1938, as illustrated in the example of FIG. 19D. At this point, the distal attachment assembly 1820 may be rotated about the static pin 1832 in a downward direction until the dynamic pin 1830 contacts the cam surface 1448. Further downward rotation causes the cam surface 1448 to urge the dynamic pin 1830 from its first position to its second position which clears the first hook 1936 allowing the dynamic pin 1830 to be received within the recess 1940.

Once the dynamic pin 1830 is received within the recess 1940, it returns to the first position or somewhere between the first and second positions thereby locking the distal attachment assembly 1820 to the distal attachment 1930 of the stabilizer 1900, as illustrated in the example of FIG. 19E.

FIGS. 19F and 19G illustrate some examples of the connection between the proximal attachment 1950 of the stabilizer 1900 and the proximal attachment assembly 1701 of the steerable guide system 1105. In this regard, once the distal attachment assembly 1820 is secured to the distal attachment 1930, the proximal attachment assembly 1701 may be lowered toward the elongate slot 1962 of the proximal attachment 1950. As the proximal attachment assembly 1701 is lowered, the fingers 1734a, 1734b and the capture members 1742 are received within the elongate slot 1962 such that the rails 1952, 1954 engage the sloped surfaces 1744 of the capture members 1742 and urge them inwardly from their first position to their second position in which their collective width is equal to or lesser than width of the fingers 1734a, 1734b. However, since the width W1 of the fingers 1734a, 1734b is slightly less than that of the elongate slot 1962, the fingers 1734a, 1734b may advance into the elongate slot 1962. Once the rails 1952, 1954 pass the shoulders 1746, the bias of the spring 1750 returns the capture members 1742 and corresponding engagement members 1740a, 1740b to their respective first position. In this configuration, the first rail 1952 and the second rail 1954 are received in their corresponding slots 1702. Additionally, the spring 1750 pushes the capture members 1742 outwardly into contact with the rails 1952, 1954 with sufficient force so as to create friction between them which limits inadvertent movement of the proximal attachment assembly 1701 along the elongate slot 1962.

Although it has been described that the proximal attachment assembly 1701 of the multi-catheter system includes the moveable engagement members 1740a, 1740b that are configured to attach to the rails 1952, 1954 and translate within the elongate slot 1962 of the proximal attachment 1950, this arrangement can be reversed. For example, the stabilizer 1900 may have a proximal attachment with engagement members, like the engagement members 1740a, 1740b, that are moveable between first and second positions, and the multi-catheter system may include a proximal attachment with rails and an elongate slot, like the rails 1952, 1954 and the elongate slot 1962, for receipt of such engagement members of the stabilizer 1900. Similarly, the configurations of the distal attachment assembly 1820 of the multi-catheter system and the distal attachment 1930 can be reversed. Thus, the stabilizer 1900 may have a distal attachment assembly with a static and dynamic pin, such as the dynamic pin 1830 and the static pin 1832, and the multi-catheter system may have a distal attachment with attachment members, like the attachment members 1932 for receiving and engaging with the static and dynamic pins.

FIGS. 19H-19I illustrate the use of the steerable guide system 1105 to position the fixation device 500 within a target valve, which is depicted as the mitral valve MV, but should be understood to alternatively be the tricuspid valve TV. In a transcatheter procedure for repairing the mitral valve MV, the distal end 1874 of the outer guide catheter 1870 is advanced through the interatrial septum S and positioned within the right atrium RA proximal to the mitral valve MV. To help position the fixation device 500 above the desired location along the line of coaptation LOC and within the valve orifice O, the inner guide catheter 1780 may be advanced from the outer guide catheter 1870. This may be achieved by sliding the proximal attachment assembly 1701 distally (i.e., forward) within the elongate slot 1962 of the proximal attachment 1950, as shown in FIG. 19H. The practitioner may utilize the distal tab 1968 as a leverage point to advance the proximal attachment assembly 1701. Should it be determined that it was advanced too far, the practitioner may slide the proximal attachment assembly 1701 proximally (i.e., backward) optionally utilizing the proximal tab 1964 as a leverage point, as shown in FIG. 19I. As illustrated in FIG. 19J, the maximum length at which the inner guide catheter 1780 can advance from the outer guide catheter 1870 is the length L2, which corresponds to the length of the elongate slot 1962. This length L2 may be determined via a statistical analysis of heart geometries of a select patient population. For example, the dimensions of the left atria LA of the select population may be analyzed up to the first, second, or third standard deviation such that when the inner guide catheter 1780 is advanced to the maximum length L2 from the outer guide catheter 1870, the fixation device 500 does not bump into and damage any structures of the atrium. Thus, the elongate slot 1962 of the proximal attachment 1950 forms a “parking spot” or “landing zone” in which, when the proximal attachment assembly 1701 is as proximal as possible (i.e., abutting the first end wall 1958), the fixation device 500 is positioned within and maintained within the distal end 1874 of the outer guide catheter 1870. This provides the operator with visual confirmation that the fixation device 500 is sheathed so that fluoroscopic confirmation is not needed which helps reduce the overall time the patient and operator are exposed to fluoroscopy and enhances patient safety. As shown in FIG. 19K, the inner guide catheter 1780 may also be curved so that the fixation device 500 may be oriented toward the mitral valve MV.

Once the fixation device 500 is positioned above the desired location along the line of coaptation LOC, the delivery catheter 850 may be advanced from the inner guide catheter 1780 into the valve. This may be achieved by unlocking the delivery catheter fastening assembly 1600 by rotating the fastener knob 1620 in the first rotation direction R1, according to some examples. The delivery system handle 1210 and the delivery catheter 850 may then be advanced along the brake shaft 1230 until the desired position within the mitral valve MV is achieved, at which point the delivery catheter fastening assembly 1600 may be locked by rotating the fastener knob 1620 in the second rotation direction R2. The maximum length at which the delivery catheter 850 can advance from the inner guide catheter 1780 is the length L1, which corresponds to the maximum distance the delivery system handle 1210 can advance along the brake shaft 1230. Again, this length L1 can be determined from a population level analysis of heart geometries to set the L1 limit. From there, the leaflets LF may be grasped. Once the leaflets LF have been sufficiently grasped and the fixation device 500 is moved to the closed position, the lock line 808 may be released, as described above. Additionally, the deployment system 1370 may be disengaged from the slider 1310 and the actuator shaft 1340. The actuator shaft 1340 may then be rotated to release the actuator rod 810 from the stud 540 or the base 532 and retracted which releases the coupling member 530 from the shaft 810 and also releases the proximal clement lines 804a, 804b from the shaft 810, as also described above.

Although the interventional system 1100 has been described as including the fixation device 500, the delivery system 1200, the steerable guide system 1105, and the stabilizer 1900, the interventional system 1100 can include more, less, or a different arrangement of components. For example, the interventional system 1100 may not include the stabilizer 1900. In another example of the interventional system 1100, the steerable guide system 1105 may be integrated into the delivery system 1200 such that the delivery catheter 850 may itself be steerable via controls integrated into the delivery catheter handle 1210. In an even further example of the interventional system 1100, the steerable guide system 1105 may alternatively be configured such that a single guide catheter provides all the steering inputs to guide the fixation device 500 to the target valve rather than the inner guide catheter assembly 1700 and the outer guide catheter assembly 1800 for performing the same. In a still further implementation, a robotically guided catheter (not shown) may be provided in the interventional system 1100 to both stabilize and steer the delivery system 1200 and the fixation device 500 to the target valve. However, in such robotically assisted procedure, a practitioner may still manually grasp leaflets and deploy the fixation device 500 via the controls of the delivery system 1200.

FIG. 10 is a schematic view of an example computing device 1000 that may be used to implement the systems and methods described in this document. The computing device 1000 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 1000 includes a processor 1010, memory 1020, a storage device 1030, a high-speed interface/controller 1040 connecting to the memory 1020 and high-speed expansion ports 1050, and a low-speed interface/controller 1060 connecting to a low-speed bus 1070 and the storage device 1030. Each of the components 1010, 1020, 1030, 1040, 1050, and 1060, are interconnected using various busses, and may be mounted on a common motherboard or in other manners as appropriate. The processor 1010 can process instructions for execution within the computing device 1000, including instructions stored in the memory 1020 or on the storage device 1030 to display graphical information for a graphical user interface (GUI) on an external input/output device, such as a display 1080 coupled to the high-speed interface 1040. In other implementations, multiple processors and/or multiple buses may be used, as appropriate, along with multiple memories and types of memory. Also, multiple computing devices 1000 may be connected, with each device providing portions of the necessary operations (e.g., as a server bank, a group of blade servers, or a multi-processor system).

The memory 1020 stores information non-transitorily within the computing device 1000. The memory 1020 may be a computer-readable medium, a volatile memory unit(s), or non-volatile memory unit(s). The non-transitory memory 1020 may be physical devices used to store programs (e.g., sequences of instructions) or data (e.g., program state information) on a temporary or permanent basis for use by the computing device 1000. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 1030 is capable of providing mass storage for the computing device 1000. In some implementations, the storage device 1030 is a computer-readable medium. In various different implementations, the storage device 1030 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer-or machine-readable medium, such as the memory 1020, the storage device 1030, or memory on the processor 1010.

The high speed controller 1040 manages bandwidth-intensive operations for the computing device 1000, while the low speed controller 1060 manages lower bandwidth-intensive operations. Such allocation of duties is exemplary only. In some implementations, the high-speed controller 1040 is coupled to the memory 1020, the display 1080 (e.g., through a graphics processor or accelerator), and to the high-speed expansion ports 1050, which may accept various expansion cards (not shown). In some implementations, the low-speed controller 1060 is coupled to the storage device 1030 and a low-speed expansion port 1090. The low-speed expansion port 1090, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 1000 may be implemented in a number of different forms, as shown in the figure. For example, it may be implemented as a standard server 1000a or multiple times in a group of such servers 1000a, as a laptop computer 1000b, or as part of a rack server system 1000c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer.

Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. An interventional system for repairing a heart valve, the interventional system comprising:
a delivery catheter having a proximal end portion and a distal end portion;
a fixation device releasably coupled to the distal end portion of the delivery catheter, the fixation device comprising:
a distal fixation element having an engagement surface and being actuatable between an open position and a closed position;
one or more reflective markers disposed on the engagement surface of the distal fixation element; and
a proximal fixation element having an engagement surface and being actuatable between:
an open position defining a graspable volume of space between the engagement surface of the proximal fixation element and the engagement surface of the distal fixation element; and
a closed position, wherein in the closed position the engagement surface of the proximal fixation element is in a position proximal to the engagement surface of the distal fixation element; and
a sensor assembly disposed on the distal end portion of the delivery catheter, the sensor assembly comprising:
a signal emitter configured to direct an emitted signal along an emission path toward the one or more reflective markers disposed on the engagement surface of the distal fixation element when in the open position; and
a signal receiver configured to receive a reflected signal of the emitted signal off of the one or more reflective markers.

2. The interventional system of claim 1, wherein the one or more reflective markers are arranged such that the reflected signal is proportional to an amount of occlusion of the one or more reflective markers by a leaflet of the heart valve positioned in the graspable volume of space.

3. The interventional system of claim 1, wherein the engagement surface of the distal fixation element has a proximal region and a distal region, the one or more reflective markers arranged in a pattern along the engagement surface of the distal fixation element from the proximal region to the distal region.

4. The interventional system of claim 1, wherein the one or more reflective markers are arranged such that the proximal fixation element fails to obstruct the emission path toward the one or more reflective markers.

5. The interventional system of claim 1, wherein the emitted signal comprises infrared light and the one or more reflective markers are configured to reflect infrared light.

6. The interventional system of claim 1, wherein the signal emitter is configured to emit near-infrared light having a wavelength of about 700 nm to about 900 nm or about 1000 nm to about 1700 nm.

7. The interventional system of claim 1, wherein the one or more reflective markers comprise a biocompatible material selected from the group consisting of gold, titanium nitride, and alumina with a metallic coating.

8. The interventional system of claim 1, further comprising a covering disposed over at least a portion of the engagement surface of the distal fixation element, the one or more reflective markers at least partially protruding through the covering.

9. The interventional system of claim 1, further comprising:
a feedback indicator; and
a controller in communication with the sensor assembly and the feedback indicator, the controller comprising a data processing hardware and memory hardware storing instructions that when executed by the data processing hardware cause the data processing hardware to:
receive signal data from the sensor assembly corresponding to the reflected signal;
determine, based on the signal data, an insertion characteristic indicative of a position of a leaflet of the heart valve in the graspable volume of space; and
present, by the feedback indicator, a representation of the insertion characteristic.

10. The interventional system of claim 9, wherein the representation of the insertion characteristic comprises at least one of a visual representation or a haptic representation.

11. A fixation device comprising:
a first clamp having a first distal element and a first proximal element, the first distal element and the first proximal element being moveable relative to each other and each having a free end defining an open end of the first clamp and a fixed end at least partially defining a closed end of the first clamp;
a second clamp having a second distal element and a second proximal element, the second distal element and the second proximal element being moveable relative to each other and each having a free end defining an open end of the second clamp and a fixed end at least partially defining a closed end of the second clamp; and
a first optical sensor disposed proximate the closed end of the first clamp, the first optical sensor having a first emitter and a first detector, the first emitter being configured to emit light in the infrared (IR) or near infrared (NIR) spectrum into a space defined between the first proximal element and the first distal element and in a direction toward the open end of the first clamp, and the first detector being configured to detect light returning from the space in a direction toward the closed end of the first clamp and to generate a signal indicative of one or more characteristics of a first tissue disposed within the first clamp.

12. The fixation device of claim 11, wherein the first emitter is configured to emit light with a wavelength selected from the range of 700 to 1,500 nm.

13. The fixation device of claim 11, wherein the one or more characteristics includes a distance between the first optical sensor and the first tissue and a tissue type.

14. The fixation device of claim 11, wherein the first emitter is configured to direct one or more beams of light toward the first proximal element such that the one or more beams of light intersect the first proximal element at one or more discrete locations.

15. The fixation device of claim 14, wherein the first detector is configured to detect light reflected back from the one or more discrete locations, and wherein the signal generated by the detector is a dynamic signal based on optical interactions between the one or more beams of light and at least one of the first proximal element and the first tissue.

16. The fixation device of claim 14, wherein the one or more beams of light include a first beam intersecting the first proximal element at a first discrete location of the one or more discrete locations.

17. The fixation device of claim 34, wherein the first discrete location is located at or beyond 50% of a grip length of the first proximal element as measured from the free end thereof.

18. The fixation device of claim 11, wherein the first emitter is oriented parallel to the first distal element when the first distal element is in an open position.

19. The fixation device of claim 11, wherein the first detector is oriented in parallel with the first emitter.

20. The fixation device of claim 11, wherein the first detector is oriented at a non-zero angle relative to the first emitter.

21. An interventional system comprising:
a delivery system having a delivery handle and a delivery catheter extending from the delivery device handle;
a fixation device having a first clamp and a second clamp each defining an open end and a closed end of the respective first and second clamps; and
an optical sensing system having a signal processing unit, a first optical sensor, and an interface communicatively coupling the first optical sensor with the signal processing unit, the first optical sensor having a first emitter and a first detector, the first emitter being configured to generate light in an infrared (IR) or near infrared (NIR) spectrum, and the first detector being configured to receive light and generate a signal indicative of one or more characteristics of a first tissue within the first clamp.

22. The system of claim 21, wherein the one or more characteristics includes a distance between the first optical sensor and the first tissue.

23. The system of claim 21, wherein the one or more characteristics includes tissue type.

24. The system of claim 21, wherein the control unit further includes an output device disposed within the delivery device handle.

25. The system of claim 24, wherein:
the delivery catheter includes a shaft having a plurality of spring arms each having first electrical terminal and an actuator rod extendable through the shaft; and
the center portion of the fixation device includes a coupling member having second electrical terminals corresponding with the first electrical terminals, wherein moving the actuator rod in a first direction urges the spring arms into engagement with the coupling member and the first electrical terminals into engagement with the second electrical terminals, and moving the actuator rod in a second direction disengages the spring arms from the coupling member and the first electrical terminals from the second electrical terminals.
